(19) United States
(12) Patent Application Publication
(10) Pub. No.: US 2013/0052165 A1
(43) Pub. Date: Feb. 28, 2013

(54) Methods of Producing Adenovirus Vectors and Viral Preparations Generated Thereby

(71) Applicants: Livnat Bangio, Petach-Tikva (IL); Naamit Sher, Rechovot (IL); Eyal Breitbart, Hashmonaim (IL)

(72) Inventors: Livnat Bangio, Petach-Tikva (IL); Naamit Sher, Rechovot (IL); Eyal Breitbart, Hashmonaim (IL)

(21) Appl. No.: 13/521,691

(22) PCT Filed: Jan. 12, 2011
(86) PCT No.: PCT/IB11/50137

§ 371 Date: Jul. 11, 2012

Related U.S. Application Data
(60) Provisional application No. 61/294,158, filed on Jan. 12, 2010.

(51) Int. Cl. C12N 15/861; C12N 7/01; A61P 35/00; C12N 7/02; A61K 35/76; A61P 9/00; C12N 7/00; C12N 7/04

(52) U.S. Cl. 424/93.6; 435/235.1; 435/236; 435/320.1; 435/239

(57) ABSTRACT

The present invention, in some embodiments thereof, relates to methods of producing adenoviruses such as pro- and anti-angiogenic adenovirus vectors and preparations generated thereby. Particularly, in some embodiments, the viral vectors comprise a heterologous pro- or anti-angiogenic gene under the transcriptional control of the murine pre-proendothelin promoter (e.g. PPE-1-3X), for targeted expression of in angiogenic endothelium.

This application claims the benefit of priority under 35 USC 119(e) of U.S. Provisional Patent Application No. 61/294,158 filed Jan. 12, 2010, the contents of which are incorporated herein by reference in their entirety.

### FIELD AND BACKGROUND OF THE INVENTION

The present invention, in some embodiments thereof, relates to methods of producing adenoviruses such as anti-angiogenic adenovirus vectors and preparations generated thereby.

Angiogenesis, the formation of new capillaries by budding from existing vessels, occurs in tumors and permits their growth, invasiveness, and the spread of metastasis. The antiangiogenic approach to antitumor treatment targets these new vessels because of their accessibility by intravenous administration, the paucity of mutations, and the amplification effect on tumor killing. The endothelial cells (ECs) of the newly formed blood vessels are affected by antiangiogenic factors, such as angiostatin and endostatin, that trigger their apoptosis. In contrast, proangiogenic factors like bFGF and VEGF contribute to cell survival. The induction of direct and specific EC apoptosis is assumed to disrupt the balance between the anti- and proapoptotic signals and to thereby cut off the tumor's blood supply.

Transductional targeting of ECs by gene therapy approaches was hampered by the inefficiency of the vascular-specific promoters used.

U.S. Pat. No. 5,747,340 teaches use of a murine endothelial cell-specific promoter which shows selectivity towards angiogenic cells, and therapeutic applications thereof.

International Application WO/2008/132729 discloses a non-replicating adenovirus vector (Ad5, E1 deleted), containing a modified murine pre-proendothelin promoter (PPE-1-3X) and a fas-chimera transgene [Fas and human tumor necrosis factor (TNF) receptor] which has been developed, in which the modified murine promoter (PPE-1-3X), is able to restrict expression of the fas chimera transgene to angiogenic blood vessels, leading to targeted apoptosis of these vessels.

Endothelial-specific gene therapy with the PPE-1-3X promoter does not increase the specificity of viral interactions with the host (e.g. transfection) but restricts the expression of the transgene to those tissues that endogenously recognize the modified promoter-angiogenic endothelial cells. The chimeric receptor can trigger the Fas pathway by binding TNFα, which is less toxic in non-tumoral tissues than using the Fas/Fas ligand mechanism, which is highly expressed in non-tumoral normal tissues such as the liver. Further, TNFα was found to be abundant in the microenvironment of tumors adding to the specificity of the transgene activity in the tumor and its surroundings. These findings suggest that the fas chimera under the regulation of the PPE-1-3X promoter (PPE-1-3X-fas-c) can be used as a potent anti tumor drug.

International Application WO2008/132729 discloses an oncolytic agent, the conditionally replicating adenovirus (CRAD) constructs under transcriptional control of the cis-acting murine pre-proendothelin promoter. Two major strategies for development of CRAD vectors have been developed, mainly focusing on the genetic engineering of the early 1 (E1) genes to restrict virus replication to target cells and to spare normal tissue. Genetic complementation-type (type 1) CRAds, such as Ad524, have a mutation in the immediately early (E1A) or early (E1B) adenoviral region, which is complemented in tumor cells but not in normal cells. In transcomplementation-type (type 2) CRAds, virus replication is controlled via a tumor/tissue-specific promoter. Placement of the adenovirus under transcriptional control of the modified preproendothelial promoter (e.g. PPE-1 3X) results in high angiogenic specificity of expression, and can be employed to provide novel and powerful solutions for the treatment of metastatic, tumor and cancer-related conditions. These constructs were proven effective in selectively inhibiting growth and development in angiogenic epithelial cells in-vitro and in treating diseases and conditions associated with excessive neovascularization in-vivo.

International Application WO2008/132729 further teaches non-replicating adenovirus vector (Ad5, E1 deleted), containing a modified murine pre-proendothelin promoter (PPE-1-3X) and a suicide transgene (thymidine kinase, TK), in which the modified murine promoter (PPE-1-3X). The “suicide gene therapy” involves the conversion of an inert prodrug into an active therapeutic agent within the cancer cells. The most widely used gene in suicide gene therapy is herpes simplex virus thymidine kinase (HSV-TK) coupled with ganciclovir (GCV). Recent studies have characterized the HSV-TK/GCV cell cytotoxicity mechanism. They revealed cell cycle arrest in the late S or G2 phase due to activation of the G2-M DNA damage checkpoint. These events were found to lead to irreversible cell death as well as a bystander effect related to cell death. Profound cell enlargement is a well-known morphological change in cells administered with the HSV-TK/GCV system. These morphological changes are due to specific cytoskeleton rearrangement. Stress actin fibers and a net of thick intermediate filaments appear following cell cycle arrest. Placement of the suicide gene under the transcriptional control of the murine pre-proendothelin promoter (PPE-1-3X) is able to restrict expression of the suicide gene to angiogenic blood vessels, leading to targeted apoptosis of these vessels.

### SUMMARY OF THE INVENTION

According to an aspect of some embodiments of the present invention there is provided a method for large scale production of an adenovirus, the method comprising: culturing in a serum-free suspension culture PER.C6 cells infected with an adenovirus which comprises a murine pre-proendothelin promoter, thereby producing the adenovirus.

According to an aspect of some embodiments of the present invention there is provided a method of producing an adenovirus, the method comprising, culturing PER.C6 cells infected with an adenovirus which comprises a murine pre-proendothelin promoter in an adherent culture under conditions suitable for viral propagation, thereby producing the adenovirus.

According to some embodiments of the present invention the adenovirus is selected from the group consisting of a non-replicating adenovirus and a conditionally replicating adenovirus.

According to some embodiments of the present invention the non-replicating adenovirus comprises a polynucleotide which comprises a fas-chimera transgene transcriptionally linked to the murine pre-proendothelin promoter.

According to some embodiments of the present invention the conditionally replicating adenovirus is transcriptionally linked to the murine pre-proendothelin promoter.

According to some embodiments of the present invention the non-replicating adenovirus comprises a polynucleotide which comprises an anti-angiogenic transgene transcriptionally linked to the murine pre-proendothelin promoter.

According to some embodiments of the present invention the non-replicating adenovirus comprises a polynucleotide which comprises a pro-angiogenic transgene transcriptionally linked to the murine pre-proendothelin promoter.

According to some embodiments of the present invention the non-replicating adenovirus comprises a polynucleotide which comprises a suicide transgene transcriptionally linked to the murine pre-proendothelin promoter.

According to some embodiments of the present invention the conditionally replicating adenovirus transcriptionally linked to the murine pre-proendothelin promoter is devoid of non-viral heterologous sequences encoding pro- or anti-angiogenic agents.

According to some embodiments of the present invention the suicide transgene comprises a thymidine kinase encoding sequence.

According to some embodiments of the present invention the adenovirus further comprises a heterologous nucleic acid sequence encoding a therapeutic agent operably linked to the murine pre-proendothelin promoter.

According to some embodiments of the present invention the heterologous nucleic acid sequence comprises an apoptotic gene.

According to some embodiments of the present invention the method of the invention further comprises recovering virus from the cells following the culturing.

According to some embodiments of the present invention the recovering is effected at a point of harvest (POH) of 3-4 days post infection and an MOI of 5.

According to some embodiments of the present invention the culturing is effected at a 5-100 L volume.

According to some embodiments of the present invention the culturing is effected at a 25 L volume.

According to some embodiments of the present invention the culturing is effected at a 50 L volume.

According to some embodiments of the present invention the culturing is effected at a 100 L volume.

According to some embodiments of the present invention the culturing is effected using a disposable bag.

According to some embodiments of the present invention the recovering is effected by subjecting the cells to a detergent lysis.

According to some embodiments of the present invention the detergent comprises Triton X-100.

According to some embodiments of the present invention the method of the invention further comprises removing cellular DNA and cell debris so as to obtain a clear feedstock.

According to some embodiments of the present invention the feedstock is subjected to Tangential Flow Filtration (TFF).

According to some embodiments of the present invention the method further comprises obtaining a viral pellet and subjecting the viral pellet to anion exchange chromatography and size exclusion chromatography.

According to some embodiments of the present invention the fas-chimera transgene comprises a polynucleotide having a nucleotide sequence as set forth in SEQ ID NO: 2.

According to some embodiments of the present invention the fas-chimera transgene comprises a polynucleotide having a nucleotide sequence as set forth in SEQ ID NO: 3.

According to some embodiments of the present invention the fas-chimera transgene comprises a polynucleotide having a nucleotide sequence as set forth in SEQ ID NO: 4.

According to some embodiments of the present invention the murine pre-pro endothelin promoter comprises a polynucleotide having a nucleotide sequence as set forth in SEQ ID NO: 5.

According to some embodiments of the present invention the murine pre-pro endothelin promoter comprises a polynucleotide having a nucleotide sequence as set forth in SEQ ID NO: 6.

According to some embodiments of the present invention the murine pre-pro endothelin promoter comprises a polynucleotide having at least two copies of the nucleotide sequence as set forth in SEQ ID NO: 6.

According to some embodiments of the present invention the murine pre-pro endothelin promoter comprises a polynucleotide having a nucleotide sequence as set forth in SEQ ID NO: 8.

According to some embodiments of the present invention the murine pre-pro endothelin promoter comprises a polynucleotide having a nucleotide sequence as set forth in SEQ ID NO: 7.

According to some embodiments of the present invention the murine pre-pro endothelin promoter comprises a polynucleotide having a nucleotide sequence as set forth in SEQ ID NO: 13.

According to some embodiments of the present invention the murine pre-pro endothelin promoter comprises a polynucleotide having a nucleotide sequence as set forth in SEQ ID NO: 12.

According to some embodiments of the present invention the non-replicating adenovirus vector is an adenovirus 5 vector.

According to some embodiments of the present invention the adenovirus 5 vector comprises a nucleic acid sequence as set forth in SEQ ID NO: 9 or 10.

According to some embodiments of the present invention the conditions comprise serum.

According to some embodiments of the present invention the recovering is effected by freeze-thaw releasing of the virus.

According to some embodiments of the present invention the method further comprises removing cellular DNA and cell debris so as to obtain a clear feedstock by ultracentrifugation.

According to some embodiments of the present invention the method further comprises centrifuging the clear feedstock on a CsCl gradient.

According to some embodiments of the present invention the method further comprises removing the CsCl using a Sephadex desalting column.

According to an aspect of some embodiments of the present invention there is provided a method for large scale production of an adenovirus, the method comprising: culturing in a serum-free suspension culture PER.C6 cells infected with an adenovirus which comprises a nucleic acid sequence as set forth in SEQ ID NO: 9 or 10, thereby producing the adenovirus.

According to an aspect of some embodiments of the present invention there is provided a method of producing an adenovirus, the method comprising, culturing PER.C6 cells infected with an adenovirus comprising a nucleic acid sequence as set forth in SEQ ID NO: 9 or 10 in an adherent culture under conditions suitable for viral propagation, thereby producing the adenovirus.

According to an aspect of some embodiments of the present invention there is provided a viral preparation generated according to the method of some embodiments of some aspects of the present invention and exhibiting an ion exchange and size exclusion chromatography traces of FIGS. 7A-B and product profile of Table 6.

According to an aspect of some embodiments of the present invention there is provided a viral preparation generated according to some embodiments of some aspects of the method of the present invention and having a product profile of Table 3.

According to an aspect of some embodiments of the present invention there is provided a pharmaceutical composition comprising as an active ingredient the viral preparation of some embodiments of some aspects of the present invention.

According to an aspect of some embodiments of the present invention there is provided a method of reducing angiogenesis in a subject in need thereof, the method comprising administering to the subject a therapeutically effective amount of the viral preparation of some embodiments of some aspects of the present invention, thereby reducing angiogenesis in the subject.

According to some embodiments of the present invention the subject has a solid tumor.

According to some embodiments of the present invention the administering comprises intravenous administration.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart schematically depicting the VB-111 production process. Indicated are cell build, virus harvest, virus purification and final fill-finish operations.

FIGS. 2A-B are flow charts for the adaptation process for PERC.6 adherent WCB. The RCB was frozen down at passage 52 which is 13 passages downstream of the WCB.

FIG. 3A is a graph showing total cell growth and viability for PerC6 infected with MVBP6111 at an MOI of 1.0 pfu per cell. Shown is the average of triplicates samples +/−SD.

FIG. 3B is a graph showing total cell growth and viability for PERC6 infected with MVBP9111 at an MOI of 2.5 pfu per cell. Shown is the average of triplicates samples +/−SD.

FIG. 3C is a graph showing total cell growth and viability for PerC6 infected with MVBP9111 at an MOI of 5.0 pfu per cell. Shown is the average of triplicates samples +/−SD.

FIG. 4 is a graph showing an immunocytochemistry (ICC) assay infectious particle titres for MOIs 1.0, 2.5 and 5.0 pfu per cell over days 2 to 3 of the culture. Shown is the average of triplicates samples +/−SD.

FIG. 5 is a graph showing HPLC assay genomic particle titres for MOIs 1.0, 2.5 and 5.0 pfu per cell over days 2 to 4 of the culture. Shown is the average of triplicates samples +/−SD.;

FIGS. 6A-B are graphs showing PER.C6 cell culture data for 5 L and 25 L Cultibag™ growth. (FIG. 6A) PER.C6 were cultured in Ex-Cell VPRO medium to exhaustion. Shown are the viable cell count, viability and population doubling times. (FIG. 6B) A 25 L CultiBag™ was cultured to a point of infection of ˜1.5E+06 viable cells/mL (indicated) and then infected with VB-111. Shown are viable cell count and viability.

FIGS. 7A-B are representative ion-exchange and size exclusion chromatography traces. (FIG. 7A-ion exchange chromatography) VB-111 was loaded after concentration and diafiltration. Virus was eluted with 500 mM NaCl as a single peak (see inlet also) with a typical OD₂₆₀/OD₂₈₀ ratio of 1.25-1.3. (FIG. 7B-size exclusion chromatography) Material eluted from the IEX column was loaded and eluted in the The OD₂₆₀/OD₂₈₀ so ratio for an Ad5 vector should be around 1.25-1.3. SDS-PAGE analysis indicates that a significant clean-up is achieved during the SEC/GPC step (FIG. 8; compare lanes 6 and 8). On completion of this step the product is concentrated to the required titers for the bulk drug substances and any further buffer exchange steps are performed at this stage.

FIG. 8 is a picture showing identity and purity analysis of in-process and final drug product material from 5 L development run. Reduced protein samples were analyzed by SDS-PAGE at the indicated process steps and compared to CsCl-double banded reference VB-111. The hexon band (most abundant protein within Ad5) is indicated; and

FIGS. 9A-B are graphs showing in-process stability at 2-8° C. Virus material was analyzed by HPLC (FIG. 9A) and ICC (FIG. 9B) for genomic and infectious titer, respectively, at 0, 24 and 48 hrs hold-time at 2-8° C. Materials were analyzed post TFF, IEX and SEC steps.

FIG. 10 is a schematic illustration showing the backbone cosmid pWE.Ad.AfAfIII-rITRsp.

FIG. 11 is a schematic illustration showing the adaptor plasmid pAdApt.

FIG. 12 is a schematic illustration showing the PPE-1-(3X)-Fas-c cassette.

FIG. 13 is a schematic illustration showing AdApt-PPE-1-3x-Fas-c with the PPE-1-3x-Fas-c gene inserts.

FIG. 14 shows a linear, schematic map of the vector AdPPE-1(3x)-TK.

FIG. 15 shows a linear, schematic map of the vector CRAd-PPE-1(3X).

### DESCRIPTION OF SPECIFIC EMBODIMENTS OF THE INVENTION

The present invention, in some embodiments thereof, relates to methods of producing adenovirus vectors such as anti-angiogenic adenovirus vectors and preparations generated thereby.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Angiogenesis is required for the development of neoplastic and hyperproliferative growths. Gene therapy for anti-angiogenic therapy in conditions associated with neovascularization, such as cancer, has been investigated, however, despite promising results in in-vitro experiments and in animal models, there has been little success with anti-angiogenic gene therapy in the clinical setting, likely due to obstacles including duration of expression of the transferred gene, induction of host immune response, cytotoxicity of the vectors and tissue specificity of expression.

The present inventors have devised a novel protocol for the production of adenoviral vectors which comprise the murine pre-proendothelin promoter. This promoter shows selectivity towards angiogenic cells and as such can be used in a myriad of therapeutic applications.

The instant specification and Examples section may put more emphasis on the production of viral vectors comprising the PPE-1-3x-Fas-c (also referred to herein as VB-111), an anti-angiogenic agent consisting of a non-replicating adenovirus vector (Ad-5, E1 and E3 deleted), which contains a modified murine pre-proendothelin promoter and a fas and human tumor necrosis factor (TNF) receptor chimeric transgene that can be readily produced in cell culture. However, by no means is the description aimed to be limiting to the production of this apoptotic agent and other therapeutic agents are also envisaged by the instant teachings.

### GENERAL TERMINOLOGY

PER.C6 refers to the continuously deviding human cell line available from Crucell™ (wwwdotcrucelldotcom). The PER.C6 cell line is distinguished from other adenovirus complementing cell lines, i.e. HER911 and HEK293, in that the E1A promoter at the 5′ end and the poly A sequence at the 3′ end of the transgene cassette have been replaced with the human Phospho Glycerate Kinase (PGK) promoter and the hepatitis B Virus (HBV) transcription termination sequence, respectively. As a result, the E1 expression cassette in the PER.C6 cell line contains only 3052 bp from Ad5 (bp 459-3510). The lack of homology between PER.C6 cell integrated E1 sequences and those in typical E1 deleted adenoviruses precludes the generation of replication competent adenovirus (RCA) via homologous recombination, thus eliminating the possibility of viral replication in the body.

As used herein, the phrase adenovirus refers to a vector in which, among the nucleic acid molecules in the viral particle, sequences necessary to function as a virus are based on the adenoviral genome.

According to a specific embodiment, the adenoviral vector is of serotype 5 (Ad5).

Adenovirus is used as a vehicle to administer targeted therapy, in the form of recombinant DNA or in this case, protein.

According to another embodiment, the adenovirus comprises a sequence as set forth in SEQ ID NO: 1 or SEQ ID NO: 11.

According to an embodiment of the invention the adenovirus is selected from the group consisting of a non-replicating adenovirus and a conditionally replicating adenovirus.

As used herein a “conditionally replicating adenovirus (CRAD)” refers to oncolytic adenoviruses which reproduce themselves in cancer cells and subsequently kill the initially infected cells by lysis. Such viruses proceed to infect adjacent cells thus repeating the cycle. According to a specific embodiment, in the CRAD vector used herein, the E1 promoter has been replaced by the modified pre-proendothelin-1 promoter PPE-1 3X, resulting in the effective reduction of viability (by 90%) of endothelial cells, without reducing viability of non-endothelial cells.

Thus, placement of the adenovirus under transcriptional control of the modified preproendothelial promoter (e.g. PPE-1 3X) results in high angiogenic specificity of expression, and can be employed to provide novel and powerful solutions for the treatment of metastatic, tumor and cancer-related conditions. Such an angiogenic specific CRAD construct can be provided in linkage with sequences of interest, as detailed hereinabove, or in the virus construct form, devoid of non-viral heterologous sequences (e.g., angiogenic or non-angiogenic).

Alternatively or additionally, the present inventors contemplate use of replication defective adenoviral vectors, such as described herein (see Example 3).

As used herein, the phrase “non-replicating virus” or “replication defective adenoviral vectors” refers to a replication-deficient viral particle, which is capable of transferring nucleic acid molecules into a host.

According to a specific embodiment the adenovirus further comprises a heterologous nucleic acid sequence encoding a therapeutic agent operably linked to said murine pre-proendothelin promoter.

Description of some embodiments of the pre-proendothelin promoter is provided below.

According to a specific embodiment, the therapeutic agent refers to a nucleic acid (e.g., silencing agent such as antisense, siRNA, ribozyme and the like) or a peptide or polypeptide product that causes cell killing i.e., cytotoxic by way of necrosis or apoptosis or at least cell growth arrest i.e., cytostatic.

According to a specific embodiment, the cytotoxic agent comprises an apoptotic gene.

Since the heterologous nucleic acid sequence is under the transcriptional control of the pre-proendothelin promoter, the therapeutic effect is on angiogenic cells where the promoter is active.

As used herein, the phrase “angiogenic cells” refers to any cells, which participate or contribute to the process of angiogenesis. Thus, angiogenic cells include but are not limited to, endothelial cells, smooth muscle cells.

In one preferred embodiment of the present invention, the expression of the therapeutic agent is directed to a subpopulation of angiogenic cells. In order to direct specific expression of a therapeutic agent in a subpopulation of angiogenic cells, the heterologous nucleic acid sequence encodes a chimeric polypeptide including a ligand binding domain which can be, for example, a cell-surface receptor domain of a receptor tyrosine kinase, a receptor serine kinase, a receptor threonine kinase, a cell adhesion molecule or a phosphatase receptor fused to an effector domain of an cytotoxic molecule such as, for example, Fas, TNFR, and TRAIL.

Such a chimeric polypeptide can include any ligand binding domain fused to any cytotoxic domain as long as activation of the ligand binding domain, i.e., via ligand binding, triggers cytotoxicity via the effector domain of the cytotoxic molecule.

Selection of the ligand binding domain and the cytotoxicity generating domain fused thereto is affected according to the type of angiogenic cell targeted for apoptosis. For example, when targeting specific subset of endothelial cells (e.g., proliferating endothelial cells, or endothelial cells exhibiting a tumorous phenotype), the chimeric polypeptide includes a ligand binding domain capable of binding a ligand naturally present in the environment of such endothelial cells and preferably not present in endothelial cells of other non-targeted tissues (e.g., TNF, VEGF). Such a ligand can be secreted by endothelial cells (autocrine), secreted by neighboring tumor cells (paracrine) or specifically targeted to these endothelial cells.

According to a specific embodiment, the chimeric polypeptide refers to the Fas-c chimera which is described in details hereinbelow. According to a specific embodiment, the viral vector comprises a non-reheating adenovirus which comprises a fas-chimera transgene transcriptionally linked to the murine pre-proendothelin promoter, as described in details below.

Alternatively, the heterologous nucleic acid agent may encode a suicide gene capable of converting a prodrug to a toxic compound.

As used herein “a suicide gene” is a nucleic acid sequence encoding for a product, wherein the product causes cell death by itself or in the presence of other compounds (prodrug). It will be appreciated that the above described construct represents only one example of a suicide construct.

According to a specific embodiment, the suicide gene refers to the herpes simplex virus thymidine kinase (HSV-TK) that when coupled with ganciclovir (GCV) administration causes cell death.

Additional examples are thymidine kinase of varicella zoster virus and the bacterial gene cytosine deaminase which can convert 5-fluorocytosine to the highly toxic compound 5-fluorouracil.

As used herein “prodrug” means any compound useful in the methods of the present invention that can be converted to a toxic product, i.e. toxic to tumor cells.

The prodrug is converted to a toxic product by the gene product of the therapeutic nucleic acid sequence (suicide gene) in the vector useful in the method of the present invention. Representative examples of such a prodrug is ganciclovir which is converted in vivo to a toxic compound by HSV-thymidine kinase. The ganciclovir derivative subsequently is toxic to tumor cells. Other representative examples of prodrugs include aciclovir, FIAU [1-(2-deoxy-2-fluoro-.beta.-D-arabinofuranosyl)-5-iodouracil], 6-methoxypurine arabinoside for VZV-TK, and 5-fluorocytosine for cytosine deambinase. Preferred suicide gene/prodrug combinations are bacteria cytosine deaminase and 5-fluorocytosine and its derivatives, varicella zoster virus TK and 6-methylpurine arabinoside and its derivatives, HSV-TK and ganciclovir, aciclovir, FIAU or their derivatives.

According to a specific embodiment, the adenovirus is a non-replicating adenovirus comprising a polynucleotide which comprises a fas-chimera transgene transcriptionally linked to the murine pre-proendothelin promoter.

According to a specific embodiment, the adenovirus is a conditionally replicating adenovirus that is transcriptionally linked to the murine pre-proendothelin promoter.

According to a specific embodiment, the adenovirus is a non-replicating adenovirus that comprises a polynucleotide which comprises a suicide transgene (e.g., thymidine kinase) transcriptionally linked to the murine pre-proendothelin promoter.

According to some embodiments of some aspects of the present invention, the heterologous nucleic acid agent may encode an pro-angiogenic agent (capable of inducing angiogenesis), or an anti-angiogenic agent (capable of inhibiting angiogenesis). According to some embodiments, the heterologous nucleic acid is a pro-angiogenic agent. Following is a non-limiting list of expressible nucleic acid sequences (genes) which are capable of inducing angiogenesis and which can be comprised in the nucleic acid construct according to some embodiments of the invention (some of which are described in Burton E R and Libutti S K. “Targeting TNF-α for cancer therapy”; Journal of Biology, 2009, Minireview, 8:85, which is fully incorporated herein by reference): Factors affecting endothelial proliferation and migration such as Vascular endothelial growth factors (VEGF family, such as VEGFA, GenBank Accession No. NM_{—}001025366.2), fibroblast growth factors (FGF family, such as FGF2 GenBank Accession No. NM_{—}002006), platelet-derived growth factor (PDGFB GenBank Accession No. NM_{—}002608), epidermal growth factor (EGF), hypoxia inducible factor (HIF1α; GenBank Accession No. NM_{—}001530), and the HIF1α triple mutant [P402A, P564G, N803A, as described in WO/2008/015675, which is incorporated fully herein by reference)].

According to some embodiments of the invention, the expressible nucleic acid sequence is capable of inhibiting angiogenesis.

Following is a non-limiting list of expressible nucleic acid sequence capable of inhibiting angiogenesis (some of which are described in Albini A., et al. “Functional genomics of endothelial cells treated with anti-angiogenic or angiopreventive drugs”. Clin. Exp. Metastasis, published online on Apr. 10, 2010, which is fully incorporated herein by reference).

Expressible nucleic acid sequences encoding toxic polypeptides or suicide polypeptides, cytotoxic pro-drug/enzymes for drug susceptibility therapy such as ganciclovir/thymidine kinase and 5-fluorocytosine/cytosine deaminase [e.g., E. coli cytosine deaminase (CD; e.g. Gene ID: 944996 nucleotides NC_{—}000913.2 (355395 . . . 356678)], herpes simplex virus thymidine kinase [TK; e.g., human herpesvirus 1 GeneID: 2703374, nucleotides NC_{—}001806.1 (46672-47802, complement)) and VEGF165B (VEGFA, GenBank Accession No. NM_{—}001025366.2);

According to some embodiments of the invention, the expressible nucleic acid sequence is capable of stabilizing, effecting and/or maturing blood vessels.

As used herein the phrase “stabilizing and/or maturing blood vessles” refers to at least enhancing the survival of endothelial cells or stroma cells (e.g., pericytes, smooth muscle cells and fibroblasts), or enhancing the interaction between endothelial cells, or between endothelial cells and stromal cells in the surrounding tissue, in a manner which reduces leakage of the blood vessel and/or extend endurance of the blood vessel resulting in appropriate and longlasting blood flow.

Non-limiting examples of expressible nucleic acid sequences which can be used to stabilize and/or mature blood vessels include platelet derived growth factor-BB (PDGFB; GenBank Accession No. NM_{—}002608; Levanon et al., Pathobiology, 2006; 73(3):149-58; also Cao et al. Nature Med. 9: 604-613, 2003) and ANGPTI.

Thus, according to an aspect of the invention there is provided a method of producing an adenovirus, the method comprising, culturing PER.C6 cells infected with an adenovirus which comprises a murine pre-proendothelin promoter in an adherent culture under conditions suitable for viral propagation, thereby producing the adenovirus.

As is illustrated hereinbelow and in the Examples section which follows, the present inventors were able to obtain highly purified viral preparations which were used in a phase I clinical trial. Specifically, adherent PER.C6 cells were expanded to T-300 cm² flasks, infected and harvested. Following clarification by freeze thaw and centrifugation, the virus was purified on CsCl gradient resulting in 30 ml, 10¹² VP/ml of purified material per batch.

As the method of this aspect of the invention uses adherent culturing conditions, the culture is initiated by seeding the PER.C6 cells and infecting the cells with the virus. The virus is propagated by incubation.

Any culture medium compatible with viral propagation can be used in accordance with the present teachings. Such media can be obtained by any commercial vendor e.g., Invitrogen™, Inc. According to a specific embodiment, the adherent cells are grown in DMEM High Glucose (Invitrogen 41966-029).

According to a specific embodiment, conditions suitable for viral propagation comprise presence of serum.

The serum can be human serum, animal serum (e.g., bovine serum or fetal calf serum) or serum replacement.

According to a specific embodiment, the culture is devoid of components from animal origin.

According to a specific embodiment, the adherent cells are grown in 10% FCS (Invitrogen 10099-141).

According to a specific embodiment culturing (infection) is effected for 72-96 hours at MOI of 5.

According to a specific embodiment, culturing is effected using 100-1000, 100-750, 200-750, 200-500, 300-500 cm² flasks, or according to a specific embodiment in 300 cm² flasks.

Once sufficient viral titre is obtained the adenovirus is recovered from the culture.

Any method known in the art can be used to release the virus from the cells. Examples include but are not limited to, detergent mediated lysis, freeze-thaw and sonication.

According to a specific embodiment, viral recovery is effected by the freeze-thaw technique.

Preferably, cell debris and host DNA are removed so as to obtain a clear feedstock.

Further purification is effected such as by using a CsCl gradient. According to a specific embodiment, the feedstock is first centrifuged on a discontinuous CsCl gradient followed by centrifugation on a continuous CsCl gradient. This is done to remove defective particles and proteins present in the cell lysate, as well as media, serum and cellular debris and to concentrate the virus to clinical applications.

According to a specific embodiment, the residual Cs is removed using a desalting column (e.g., two rounds of Sephadex desalting columns).

Harvests may be pulled at this point to produce a larger batch, following appropriate testing as further described hereinbelow.

The virus is eluted from the column such as by using PBS.

Finally, the virus is diluted to the required concentration (vp/ml) with a solution of PBS including glycerol e.g., 10%.

According to a further embodiment the composition is sterile filtrated and put into vials for storage. The final product is stored at −65° C. or less.

A viral preparation generated according to this method is also contemplated according to the present teachings.

According to an exemplary embodiment, the viral preparation comprises between 0-200, 0-150, 5-200 or 5-150 μg/L Cs, as assayed by mass spectrometry.

According to another exemplary embodiment, the viral preparation comprises about 5 μg/L Cs or less, as assayed by mass spectrometry.

Below is a summary of methods that can be used for characterization, in process, release and stability testing of the viral preparation manufactured in adherent cells grown with serum (Tables 1-2).

TABLE 1
Methods Used For Batch Release (VB-111 manufactured in
adherent cells grown with serum)

| Fraction Tested | Parameter |
|---|---|
| Harvest | Identity by PCR |
| | Microbial Limit |
| | ADA (In-vitro adventitious agents) |
| | Mycoplasma (indicator DNA fluorochrome test and |
| | cultivation assay) |
| Puridied | RCA (Detection of replication competent |
| bulk | Adenovirus using the A549 detector cell line) |
| (PAG) | Host cell DNA residues (qPCR) |
| Final product | Cs residues |
| | Sterility |
| | Endotoxins (chromogenic assay) |
| | Appearance |
| | Potency by Plaque forming unit (pfu) |
| | Transgene expression by Western blot |
| calculated | vp/pfu ratio |

*In reference to PAG (Purified bulk after addition of Glycerol)

TABLE 2:
Methods Used for In Process Testing (VB-111 manufactured in adherent
cells grown with serum)

| Fraction Tested | Parameter |
|---|---|
| Harvest | Mycoplasma (by PCR) |
| (only prior to pooling) | Microbial Limit |
| Harvest | Mycoplasma, (indicator DNA fluorochrome |
| (single or Pooled samples) | test and cultivation assay) |
| | Microbial Limit |
| | ADA (In-vitro adventitious agents) |
| Initial clarified harvest | Identity by PCR |
| | Plaque fomting unit (pfu) |
| Purified Bulk | Viral particles (OD ₂₆₀) |

Table 3 below, provides an embodiment of the viral final product as grown in PER.C6 cells under adherent conditions.

TABLE 3
Product specifications (VB-111 manufactured in adherent cells
grown with serum)

| Parameter | Sampled From | Specifications |
|---|---|---|
| Appearance | Final product | White or Colorless |
| Identity | | |
| PCR(using the below primers | Initially clarified | Co-migration in gel with |
| (p55 and ppe) | Harvest | positive control |
| Quantitation | | |
| Viral particles (OD₂₆₀) | Final product | According to expected |
| | | dilution* |
| Potency | | |
| Plaque forming unit (pfu) | Final product | ≧1 × 10⁹ pfu/ml |
| Transgene expression (Western | Final product | Positive |
| blot) | | |
| vp/pfu ratio | calculated | ≦30 |
| Impurities | . | |
| Microbial Limit | Harvest | ≦10 CFU/ml |
| ADA (In-vitro adventitious | Harvest | Negative |
| agents) | | |
| Mycoplasma (indicator DNA | Harvest | Negative |
| fluorochrome test and | | |
| cultivation assay) | | |
| RCA | Purified bulk (PAG) | <1 RCA/3 × 10¹⁰ vp |
| Host cell DNA residues | Purified bulk | For information only |
| | (PAG) | |
| Csresidues | Final product | values are provided below |
| Sterility | Final product | Negative (no |
| | | contamination) |
| Endotoxins (chromogenic | Final product | ≦350 EU/ dose |
| assay) | | |

*In reference to PAG (Purified bulk after addition of Glycerol)

While further reducing the present invention to practice and in order to introduce the PPE-1-3x-Fas-c chimera into commercial use in the clinic, the present inventors have developed scaled-up process, to support production of larger quantities needed for phase II/III clinical studies and actual therapy. This production is aimed to provide purity that is at least comparable to that achieved with high resolution laboratory processes such as CsCl banding. Such a production process allows manufacture of high titers of materials for early/late stage clinical trials and commercial supply.

As is detailed in the Examples section which follows, the scaled-up production process was adapted to serum-free production using a suspended cell culture where earlier production protocols involved the use of adherent cells grown in serum. The revised process as exemplified in the examples section uses 50 liter disposable CultiBags (Wave) for the upstream production and chromatography steps for the down stream purification.

Using the novel production process the present inventors were able to achieve a viral titre of 10¹⁰-10¹¹/mL of crude harvest, making the production of material for clinical trials even at high dose levels achievable in relatively small scale production facilities. In turn these production scales also allow for the newly emerging disposable systems to be used in its production.

Thus, according to as aspect of the invention, there is provided a method for large scale-production of a specific non-replicating adenovirus vector, the method comprising, culturing PER.C6 cells infected with a non-replicating adenovirus vector in a serum-free suspension culture, the vector comprising a polynucleotide which comprises a fas-chimera transgene transcriptionally linked to a murine pre-proendothelin promoter, thereby producing the specific non-replicating adenovirus vector.

As used herein the phrase “large-scale production” refers to at least 100 ml batch production (starting with a culture volume of 5-100 L), which results in a viral quantity of at least 1×10¹² virus particles/ml and a viral potency of at least 3×10¹° Pfu/ml.

Culture volume refers to the volume of the culture medium, that is typically half that of the culture bag used.

As used herein the term “serum-free” refers to a culture medium which is absent of serum and as such its components are highly defined.

The use of serum-free medium is highly advantageous since it is endowed with increased definition, consistent performance, easier purification and downstream processing, precise evaluations of cellular function, increased growth and/or productivity, better control over physiological responsiveness.

The medium may still include the addition of growth factors and/or cytokines. According to an exemplary embodiment HEPES and Glutamine are added to the culture. According to a specific embodiment, the following conditions can be sed: Ex-cell VPRO medium (Sigma 14561C), 1M HEPES Buffer pH 7.0-7.6 using 6 mM in medium (Sigma H0887), Glutamax using 10 mM in medium (Invitrogen 35050)

As used herein the term “serum” refers to human or animal serum.

According to a specific embodiment, the culture is devoid of components from animal origin.

As mentioned the viral vectors of this aspect of the present invention comprise a cytotoxic fas-chimera effector sequence under transcriptional control of an angiogenic endothelial-specific modified murine pre-pro endothelin promoter.

Typically, such viral vectors are constructed using genetic recombination technology—i.e. recombinant viral vectors.

The Fas-chimera (Fas-c) polypeptide, is a previously described fusion of two “death receptors”, constructed from the extracellular region of TNFR1 (SEQ ID NO: 2) and the trans-membrane and intracellular regions of Fas (SEQ ID NO: 3) [Boldin M P et al. J Biol Chem (1995) 270(14):7795-8; the contents of which are incorporated herein by reference].

According to one embodiment the Fas-c is encoded by a polynucleotide as set forth in SEQ ID NO: 4.

The term “promoter” as used herein refers to a DNA sequence which directs transcription of a polynucleotide sequence operatively linked thereto in the cell in a constitutive or inducible manner. The promoter may also comprise enhancer elements which stimulate transcription from the linked promoter.

The pre-pro endothelial promoter as used herein refers to the preproendothelin-1 (PPE-1) promoter, of mammalian origin. In one embodiment, the pre-proendothelin 1 promoter is a murine pre-pro endothelin 1 promoter (PPE-1, SEQ ID NO: 13) and modifications thereof.

According to one embodiment the promoter comprises at least one copy of an enhancer element that confers endothelial cell specific transcriptional activity. According to one embodiment the enhancer element is naturally found positioned between the −364 bp and −320 bp of the murine PPE-1 promoter (as set forth in SEQ ID NO: 6). In one embodiment, the promoter comprises at least two and more preferably three of the above described enhancer elements. According to a specific embodiment, the promoter comprises two of the above described enhancer elements on one strand of the promoter DNA and one of the above described enhancer element on the complementary strand of the promoter DNA.

In yet another embodiment, the promoter comprises a modified enhancer element as set forth in SEQ ID NO: 8, optionally in combination with other enhancer elements. Thus, according to this embodiment, the promoter comprises a sequence as set forth in SEQ ID NO: 7.

According to another embodiment, the promoter further comprises at least one hypoxia response element—e.g. comprising a sequence as set forth in SEQ ID NO: 5. An exemplary promoter which can be used in the context of the present invention comprises a sequence as set forth in SEQ ID NO: 12. This sequence comprises SEQ ID NO: 5 and SEQ ID NO: 7 (which itself comprises two copies of SEQ ID NO: 6 either side of one copy of SEQ ID NO: 8).

According to a particular embodiment of this aspect of the present invention, the viral vector consists of a sequence as set forth in SEQ ID NOs: 9 or 10.

The Ad5-PPE-1-3X-fas-c sequence, as set forth in SEQ ID NO: 9 or 10 comprises a sequence which is an anti-sense copy of SEQ ID NO: 7, located at nucleic acid coordinates 894-1036, a sequence which is a single antisense copy of SEQ ID NO: 8 located at nucleotide coordinates 951-997; a sequence which is a first antisense copy of SEQ ID NO: 6 located at nucleotide coordinates 907-950; a sequence which is a second antisense copy of SEQ ID NO: 6 located at nucleotide coordinates 993-1036; and a third copy of SEQ ID NO: 6 in the sense orientation at position 823-866.

In some embodiments of the invention, the viral vector comprises additional polynucleotide sequences capable of enhancing or inhibiting transcriptional activity of an endothelial specific promoter. According to an aspect of some embodiments of the invention, the additional polynucleotide sequence includes an, isolated polynucleotide comprising at least 6 nucleotides of element X of a pre-proendothelin (PPE-1) promoter, the element X having a wild type sequence as set forth by SEQ ID NO:6, wherein the at least 6 nucleotides comprise at least 2 consecutive sequences derived from SEQ ID NO:6, each of the at least 2 consecutive sequences comprises at least 3 nucleotides, at least one of the at least 3 nucleotide being positioned next to at least one nucleotide position in SEQ ID NO:6, the at least one nucleotide position in SEQ ID NO:6 is selected from the group consisting of:

(i) at least one nucleotide of wild type M4 sequence set forth by SEQ ID NO: 15 (CATTC);

(ii) at least one nucleotide of wild type M5 sequence set forth by SEQ ID NO: 16 (CAATG);

(iii) at least one nucleotide of wild type M8 sequence set forth by SEQ ID NO: 19 (GCTTC);

(iv) at least one nucleotide of wild type M6 sequence set forth by SEQ ID NO: 17 (GGGTG);

(v) at least one nucleotide of wild type M7 sequence set forth by SEQ ID NO: 18 (ACTTT);

(vi) at least one nucleotide of wild type M1 sequence set forth by SEQ ID NO: 20 (GTACT); and

(v) at least one nucleotide of wild type M3 sequence set forth by SEQ ID NO: 21 (CTTTT);

wherein the at least one nucleotide position is mutated as compared to SEQ ID NO:6 by at least one nucleotide substitution, at least one nucleotide deletion and/or at least one nucleotide insertion, with the proviso that a mutation of the at least one nucleotide position does not result in nucleotides GGTA at position 21-24 of SEQ ID NO:6 and/or in nucleotides CATG at position 29-32 of SEQ ID NO:6, such that when the isolated polynucleotide is integrated into the PPE-1 promoter and placed upstream of a reporter gene (e.g., luciferase coding sequence) the expression level of the reporter gene is upregulated or downregulated as compared to when SEQ ID NO:6 is similarly integrated into the PPE-1 promoter and placed upstream of the reporter gene coding sequence.

According to some embodiments of the invention, the isolated polynucleotide is not naturally occurring in a genome or a whole chromosome sequence of an organism.

As used herein the phrase “naturally occurring” refers to as found in nature, without any man-made modifications.

As described above, the at least 6 nucleotides of element X comprise at least 2 consecutive sequences derived from SEQ ID NO:6.

As used herein the phrase “consecutive sequence derived from SEQ ID NO:6” refers to a nucleic acid sequence (a polynucleotide) in which the nucleotides appear in the same order as in the nucleic acid sequence of SEQ ID NO:6 from which they are derived. It should be noted that the order of nucleotides is determined by the chemical bond (phosphodiester bond) formed between a 3′-OH of a preceding nucleotide and the 5′-phosphate of the following nucleotide.

According to some embodiments of the invention, each of the at least 2 consecutive sequences comprises at least 3 nucleotides, e.g., 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotide, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides of SEQ ID NO:6.

As described, the isolated polynucleotide comprises at least 2 consecutive sequences derived from SEQ ID NO:6. According to some embodiments of the invention, the isolated polynucleotide comprises 2, 3, 4, 5, 6, 7, 8, 9, 10, 11, 12, 13 or 14 consecutive sequences derived from SEQ ID NO:6.

As used herein the phrase “wild type” with respect to a nucleotide sequence refers to the nucleic acid sequence as appears in SEQ ID NO:6. Examples include, but are not limited to wild type M4 sequence (SEQ ID NO: 15), wild type M5 sequence (SEQ ID NO: 16), wild type M8 (SEQ ID NO:19), wild type M6 sequence (SEQ ID NO:17), wild type M7 sequence (SEQ ID NO:18), wild type M1 (SEQ ID NO:20) and wild type M3 sequence (SEQ ID NO:21).

According to some embodiments of the invention, the mutation is an insertion of at least one nucleotide in a nucleotide position with respect to SEQ ID NO:6. According to some embodiments of the invention, the insertion includes at least 1, 2, 3, 4, 5, 6, 7, 8, 9, 10 nucleotides, e.g., at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, or more nucleotides.

It should be noted that the sequence which is inserted by the mutation can be derived from any source (e.g., species, tissue or cell type), and is not limited to the source of the sequence of element X.

According to some embodiments of the invention, the mutation is a combination of any of the mutation types described above, i.e., substitution, insertion and deletion. For example, while one nucleotide position in SEQ ID NO:6 can be subject to a substitution mutation, another nucleotide position in SEQ ID NO:6 can be subject to a deletion or insertion. Additionally or alternatively, while one nucleotide position in SEQ ID NO:6 can be subject to a deletion mutation, another nucleotide position in SEQ ID NO:6 can be subject to a substitution or insertion. Additionally or alternatively, while one nucleotide position in SEQ ID NO:6 can be subject to an insertion mutation, another nucleotide position in SEQ ID NO:6 can be subject to a substitution or deletion. It should be noted that various other combinations are possible.

According to specific embodiments of the invention, the mutation in the isolated polynucleotide of the invention does not result in nucleotides GGTA at position 21-24 of SEQ ID NO:6 and/or in nucleotides CATG at position 29-32 of SEQ ID NO:6.

As used herein the phrase “integrated into the PPE-1 promoter” refers to a nucleotide sequence (the isolated polynucleotide) which is covalently conjugated within the PPE-1 promoter sequence.

According to some embodiments of the invention, the isolated polynucleotide further comprises at least one copy of a nucleic acid sequence selected from the group consisting of:

| (i) | wild type M4 sequence set forth by SEQ ID NO: 15 (CATTC), |
|---|---|
| (ii) | wild type M5 sequence set forth by SEQ ID NO: 16 (CAATG), |
| (iii) | wild type M8 sequence set forth by SEQ ID NO: 19 (GCTTC), |
| (iv) | wild type M6 sequence set forth by SEQ ID NO: 17 (GGGTG), |
| (v) | wild type M7 sequence set forth by SEQ ID NO: 18 (ACTTT); |
| (vi) | wild type M1 sequence set forth by SEQ ID NO: 20 (GTACT), |
| and | |
| (vii) | wild type M3 sequence set forth by SEQ ID NO: 21 (CTTTT). |

According to some embodiments of the invention, the isolated polynucleotide is integrated into (within), downstream of, or upstream of any known (or unknown) promoter sequence to thereby regulate (e.g., increase, decrease, modulate tissue-specificity, modulate inductive or constitutive expression) the transcriptional promoting activity of the promoter.

According to some embodiments of the invention, the isolated polynucleotide is for increasing expression of a heterologous polynucleotide operably linked thereto in endothelial cells. Such a polynucleotide can include wild type sequences of M4 and/or M5 in the presence or absence of additional sequences from element X, and/or in the presence of other mutated sequences from element X.

According to some embodiments of the invention, the isolated polynucleotide comprises at least one copy of the wild type M4 sequence set forth by SEQ ID NO: 15 (CATTC).

According to some embodiments of the invention, the isolated polynucleotide comprises at least one copy of the wild type M5 sequence set forth by SEQ ID NO: 16 (CAATG).

According to some embodiments of the invention, the isolated polynucleotide comprises at least one copy of the wild type M4 sequence set forth by SEQ ID NO: 15 (CATTC) and at least one copy of the wild type M5 sequence set forth by SEQ ID NO: 16 (CAATG).

According to some embodiments of the invention, the at least one nucleotide position which is mutated as compared to SEQ ID NO:6 is at least one nucleotide of the wild type M8 sequence set forth by SEQ ID NO: 19 (GCTTC). It should be noted that such an isolated polynucleotide may further include a wild type M6 sequence (SEQ ID NO:17) and/or a wild type M7 sequence (SEQ ID NO:18)

Non-limiting examples of isolated polynucleotides which include at least one copy of the wild type M4 sequence set forth by SEQ ID NO: 15 (CATTC) and a mutation in at least one nucleotide of the wild type M8 sequence set forth by SEQ ID NO: 19 (GCTTC) are provided in SEQ ID NOs:55-62.

Non-limiting examples of isolated polynucleotides which include at least one copy of the wild type M5 sequence set forth by SEQ ID NO: 16 (CAATG) and a mutation in at least one nucleotide of the wild type M8 sequence set forth by SEQ ID NO: 19 (GCTTC) are provided in SEQ ID NOs: 63-66.

Non-limiting examples of isolated polynucleotides which include at least one copy of the wild type M4 sequence set forth by SEQ ID NO: 15 (CATTC), at least one copy of the wild type M5 sequence set forth by SEQ ID NO: 16 (CAATG) and a mutation in at least one nucleotide of the wild type M8 sequence set forth by SEQ ID NO: 19 (GCTTC) are provided in SEQ ID NOs: 67-70.

According to some embodiments of the invention, the isolated polynucleotide further comprising at least one copy of wild type M1 sequence set forth by SEQ ID NO: 20 (GTACT).

Non-limiting examples of isolated polynucleotides which include at least one copy of the wild type M4 sequence set forth by SEQ ID NO: 15 (CATTC), at least one copy of the wild type M1 sequence set forth by SEQ ID NO: 20 (GTACT), and a mutation in at least one nucleotide of the wild type M8 sequence set forth by SEQ ID NO: 19 (GCTTC) are provided in SEQ ID NOs: 71-105.

Non-limiting examples of isolated polynucleotides which include at least one copy of the wild type M5 sequence set forth by SEQ ID NO: 16 (CAATG), at least one copy of the wild type M1 sequence set forth by SEQ ID NO: 20 (GTACT) and a mutation in at least one nucleotide of the wild type M8 sequence set forth by SEQ ID NO: 19 (GCTTC) are provided in SEQ ID NOs: 106-136.

Non-limiting examples of isolated polynucleotides which include at least one copy of the wild type M4 sequence set forth by SEQ ID NO: 15 (CATTC), at least one copy of the wild type M5 sequence set forth by SEQ ID NO: 16 (CAATG), at least one copy of the wild type M1 sequence set forth by SEQ ID NO: 20 (GTACT) and a mutation in at least one nucleotide of the wild type M8 sequence set forth by SEQ ID NO: 19 (GCTTC) are provided in SEQ ID NOs:137-152.

According to some embodiments of the invention, the isolated polynucleotide reduces expression of a heterologous polynucleotide operably linked thereto in endothelial cells. Such a polynucleotide can include mutations in M4 and/or M5 in the presence or absence of additional sequences from element X, and/or in the presence of other mutated sequences from element X.

According to some embodiments of the invention, the at least one nucleotide position which is mutated as compared to SEQ ID NO:6 is at least one nucleotide of the wild type M4 sequence set forth by SEQ ID NO: 15 (CATTC).

Non-limiting examples of isolated polynucleotides which includes a mutation in at least one nucleotide of the wild type M4 sequence set forth by SEQ ID NO:46 (CATTC) are provided in SEQ ID NOs:153-162.

According to some embodiments of the invention, the at least one nucleotide position which is mutated as compared to SEQ ID NO:6 is at least one nucleotide of the wild type M5 sequence set forth by SEQ ID NO: 16 (CAATG).

Non-limiting examples of isolated polynucleotides which include a mutation in at least one nucleotide of the wild type M5 sequence set forth by SEQ ID NO: 16 (CAATG) are provided in SEQ ID NOs:163-171.

According to some embodiments of the invention, the at least one nucleotide position which is mutated as compared to SEQ ID NO:6 is at least one nucleotide of the wild type M4 sequence set forth by SEQ ID NO: 15 (CATTC) and at least one nucleotide of the wild type M5 sequence set forth by SEQ ID NO: 16 (CAATG).

Non-limiting examples of isolated polynucleotides which include a mutation in is at least one nucleotide of the wild type M4 sequence set forth by SEQ ID NO: 15 (CATTC) and a mutation in at least one nucleotide of the wild type M5 sequence set forth by SEQ ID NO: 16 (CAATG) are provided in SEQ ID NOs:172-180.

According to some embodiments of the invention, the isolated polynucleotide is for increasing expression of a heterologous polynucleotide operably linked thereto in cells other than endothelial cells. Such a polynucleotide can include mutations in M4 and/or M5 and wild type sequences of M6 and/or M7, in the presence or absence of additional sequences from element X, and/or in the presence of other mutated sequences from element X.

According to some embodiments of the invention, the isolated polynucleotide comprises a mutation in M4 (SEQ ID NO: 15) and/or in M5 (SEQ ID NO 16) and at least one copy of the wild type M6 set forth by SEQ ID NO: 17 (GGGTG) and/or at least one copy of wild type M7 set forth by SEQ ID NO:18.

Non-limiting examples of isolated polynucleotides which include a mutation in at least one nucleotide of the wild type M4 sequence set forth by SEQ ID NO: 15 (CATTC) and at least one copy of the wild type M6 set forth by SEQ ID NO: 17 (GGGTG) are provided in SEQ ID NOs:181-182.

Non-limiting examples of isolated polynucleotides which include a mutation in at least one nucleotide of the wild type M5 sequence set forth by SEQ ID NO: 16 (CAATG) and at least one copy of the wild type M6 set forth by SEQ ID NO: 17 (GGGTG) are provided in SEQ ID NOs:183-189. Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG) and at least one copy of the wild type M6 set forth by SEQ ID NO: 17 (GGGTG) are provided in SEQ ID NOs:190-191. According to some embodiments of the invention, the isolated polynucleotide further comprises at least one copy of the wild type M7 sequence set forth by SEQ ID NO: 18 (ACTTT).

Non-limiting examples of isolated polynucleotides which include a mutation in at least one nucleotide of the wild type M4 sequence set forth by SEQ ID NO: 15 (CATTC) and at least one copy of the wild type M7 sequence set forth by SEQ ID NO: 18 (ACTTT) are provided in SEQ ID NOs:192-195.

Non-limiting examples of isolated polynucleotides which include a mutation in at least one nucleotide of the wild type M5 sequence set forth by SEQ ID NO: 16 (CAATG) and at least one copy of the wild type M7 sequence set forth by SEQ ID NO: 18 (ACTTT) are provided in SEQ ID NOs:196-198.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG) and at least one copy of the wild type M7 sequence set forth by SEQ ID NO: 18 (ACTTT) are provided in SEQ ID NOs:199-202.

According to some embodiments of the invention, the isolated polynucleotide further comprises at least one copy of the wild type M6 set forth by SEQ ID NO: 17 (GGGTG) and at least one copy of the wild type M7 sequence set forth by SEQ ID NO: 18 (ACTTT).

Non-limiting examples of isolated polynucleotides which include a mutation in at least one nucleotide of the wild type M4 sequence set forth by SEQ ID NO: 15 (CATTC), at least one copy of the wild type M6 set forth by SEQ ID NO: 17 (GGGTG) and at least one copy of the wild type M7 sequence set forth by SEQ ID NO: 18 (ACTTT) are provided in SEQ ID NOs:203-205.

Non-limiting examples of isolated polynucleotides which include a mutation in at least one nucleotide of the wild type M5 sequence set forth by SEQ ID NO: 16 (CAATG), at least one copy of the wild type M6 set forth by SEQ ID NO: 17 (GGGTG) and at least one copy of the wild type M7 sequence set forth by SEQ ID NO: 18 (ACTTT) are provided in SEQ ID NOs:206-207.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M6 set forth by SEQ ID NO: 17 (GGGTG) and at least one copy of the wild type M7 sequence set forth by SEQ ID NO: 18 (ACTTT) are provided in SEQ ID NOs:208-209.

According to some embodiments of the invention, the isolated polynucleotide reduces expression in cells of a heterologous polynucleotide operably linked thereto. Such a polynucleotide can include mutations in M4, M5, M6 and/or M7, in the presence or absence of additional sequences from element X, and/or in the presence of other mutated sequences from element X.

According to some embodiments of the invention, the isolated polynucleotide comprises at least one mutation in wild type M4 (SEQ ID NO: 15) and/or in wild type M5 (SEQ ID NO:47) and in wild type M6 set forth by SEQ ID NO: 17 (GGGTG).

Non-limiting examples of isolated polynucleotides which include a mutation in at least one nucleotide of the wild type M4 sequence set forth by SEQ ID NO: 15 (CATTC) and a mutation in at least one nucleotide position of the wild type M6 set forth by SEQ ID NO: 17 (GGGTG) are provided in SEQ ID NOs:210-213.

Non-limiting examples of isolated polynucleotides which include a mutation in at least one nucleotide of the wild type M5 sequence set forth by SEQ ID NO: 16 (CAATG) and a mutation in at least one nucleotide position of the wild type M6 set forth by SEQ ID NO: 17 (GGGTG) are provided in SEQ ID NOs:214-222.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), and a mutation in at least one nucleotide position of the wild type M6 set forth by SEQ ID NO: 17 (GGGTG) are provided in SEQ ID NOs:223-231.

According to some embodiments of the invention, the isolated polynucleotide further comprises at least one mutation in wild type M7 set forth by SEQ ID NO: 18 (ACTTT).

Non-limiting examples of isolated polynucleotides which include a mutation in at least one nucleotide of the wild type M4 sequence set forth by SEQ ID NO: 15 (CATTC) and a mutation in at least one nucleotide position of the wild type M7 set forth by SEQ ID NO: 18 (ACTTT) are provided in SEQ ID NOs:232-236.

Non-limiting examples of isolated polynucleotides which include a mutation in at least one nucleotide of the wild type M5 sequence set forth by SEQ ID NO: 16 (CAATG) and a mutation in at least one nucleotide position of the wild type M7 set forth by SEQ ID NO: 18 (ACTTT) are provided in SEQ ID NOs:237-240.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), and a mutation in at least one nucleotide position of the wild type M7 set forth by SEQ ID NO: 18 (ACTTT) are provided in SEQ ID NOs:241-248.

According to some embodiments of the invention, the isolated polynucleotide further comprises at least one mutation in wild type M6 set forth by SEQ ID NO: 17 (GGGTG) and at least one mutation in wild type M7 set forth by SEQ ID NO: 18 (ACTTT).

Non-limiting examples of isolated polynucleotides which include a mutation in at least one nucleotide of the wild type M4 sequence set forth by SEQ ID NO: 15 (CATTC), a mutation in at least one nucleotide position of the wild type M6 set forth by SEQ ID NO: 17 (GGGTG) and a mutation in at least one nucleotide position of the wild type M7 set forth by SEQ ID NO: 18 (ACTTT) are provided in SEQ ID NOs:249-258.

Non-limiting examples of isolated polynucleotides which include a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG) and a mutation in at least one nucleotide position of the wild type M7 set forth by SEQ ID NO: 18 (ACTTT) are provided in SEQ ID NOs:259-264.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG) and a mutation in at least one nucleotide position of the wild type M7 set forth by SEQ ID NO: 18 (ACTTT) are provided in SEQ ID NOs:265-270.

According to some embodiments of the invention, the isolated polynucleotide comprises at least one copy of the wild type M8 sequence set forth by SEQ ID NO: 19 (GCTTC) with additional wild type or mutated sequences derived from element X (SEQ ID NO:6).

Non-limiting examples of isolated polynucleotides which includes a mutation in at least one nucleotide of the wild type M4 sequence set forth by SEQ ID NO: 15 (CATTC) and at least one copy of the wild type M8 sequence set forth by SEQ ID NO: 19 (GCTTC) are provided in SEQ ID NOs:271-279.

Non-limiting examples of isolated polynucleotides which include a mutation in at least one nucleotide of the wild type M5 sequence set forth by SEQ ID NO: 16 (CAATG) and at least one copy of the wild type M8 sequence set forth by SEQ ID NO: 19 (GCTTC) are provided in SEQ ID NOs:280-287.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG) and at least one copy of the wild type M8 sequence set forth by SEQ ID NO: 19 (GCTTC) are provided in SEQ ID NOs:288-291.

Non-limiting examples of isolated polynucleotides which include a mutation in at least one nucleotide of the wild type M4 sequence set forth by SEQ ID NO: 15 (CATTC), at least one copy of the wild type M6 set forth by SEQ ID NO: 17 (GGGTG) and at least one copy of the wild type M8 sequence set forth by SEQ ID NO: 19 (GCTTC) are provided in SEQ ID NOs:294-298.

Non-limiting examples of isolated polynucleotides which include a mutation in at least one nucleotide of the wild type M5 sequence set forth by SEQ ID NO: 16 (CAATG), at least one copy of the wild type M6 set forth by SEQ ID NO: 17 (GGGTG) and at least one copy of the wild type M8 sequence set forth by SEQ ID NO: 19 (GCTTC) are provided in SEQ ID NOs:299-301.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M6 set forth by SEQ ID NO: 17 (GGGTG) and at least one copy of the wild type M8 sequence set forth by SEQ ID NO: 19 (GCTTC) are provided in SEQ ID NOs:302-303.

Non-limiting examples of isolated polynucleotides which include a mutation in at least one nucleotide of the wild type M4 sequence set forth by SEQ ID NO: 15 (CATTC), at least one copy of the wild type M7 sequence set forth by SEQ ID NO: 18 (ACTTT) and at least one copy of the wild type M8 sequence set forth by SEQ ID NO: 19 (GCTTC) are provided in SEQ ID NOs:304-308.

Non-limiting examples of isolated polynucleotides which include a mutation in at least one nucleotide of the wild type M5 sequence set forth by SEQ ID NO: 16 (CAATG), at least one copy of the wild type M7 sequence set forth by SEQ ID NO: 18 (ACTTT) and at least one copy of the wild type M8 sequence set forth by SEQ ID NO: 19 (GCTTC) are provided in SEQ ID NOs:309-311.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M7 sequence set forth by SEQ ID NO: 18 (ACTTT) and at least one copy of the wild type M8 sequence set forth by SEQ ID NO: 19 (GCTTC) are provided in SEQ ID NOs:312-315.

Non-limiting examples of isolated polynucleotides which include a mutation in at least one nucleotide of the wild type M4 sequence set forth by SEQ ID NO: 15 (CATTC), at least one copy of the wild type M6 set forth by SEQ ID NO: 17 (GGGTG), at least one copy of the wild type M7 sequence set forth by SEQ ID NO: 18 (ACTTT) and at least one copy of the wild type M8 sequence set forth by SEQ ID NO: 19 (GCTTC) are provided in SEQ ID NO:316.

Non-limiting examples of isolated polynucleotides which include a mutation in at least one nucleotide of the wild type M5 sequence set forth by SEQ ID NO: 16 (CAATG), at least one copy of the wild type M6 set forth by SEQ ID NO: 17 (GGGTG), at least one copy of the wild type M7 sequence set forth by SEQ ID NO: 18 (ACTTT) and at least one copy of the wild type M8 sequence set forth by SEQ ID NO: 19 (GCTTC) are provided in SEQ ID NO:317.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M6 set forth by SEQ ID NO: 17 (GGGTG), at least one copy of the wild type M7 sequence set forth by SEQ ID NO: 18 (ACTTT) and at least one copy of the wild type M8 sequence set forth by SEQ ID NO: 19 (GCTTC) are provided in SEQ ID NO:318.

Non-limiting examples of isolated polynucleotides which include a mutation in at least one nucleotide of the wild type M4 sequence set forth by SEQ ID NO: 15 (CATTC), a mutation in at least one nucleotide position of the wild type M6 set forth by SEQ ID NO: 17 (GGGTG) and at least one copy of the wild type M8 sequence set forth by SEQ ID NO: 19 (GCTTC) are provided in SEQ ID NOs:319-327.

Non-limiting examples of isolated polynucleotides which include a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG) and at least one copy of the wild type M8 sequence set forth by SEQ ID NO: 19 (GCTTC) are provided in SEQ ID NOs:328-333.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG) and at least one copy of the wild type M8 sequence set forth by SEQ ID NO: 19 (GCTTC) are provided in SEQ ID NOs:334-337.

Non-limiting examples of isolated polynucleotides which include a mutation in at least one nucleotide of the wild type M4 sequence set forth by SEQ ID NO: 15 (CATTC), a mutation in at least one nucleotide position of the wild type M7 set forth by SEQ ID NO: 18 (ACTTT) and at least one copy of the wild type M8 sequence set forth by SEQ ID NO: 19 (GCTTC) are provided in SEQ ID NOs:338-344.

Non-limiting examples of isolated polynucleotides which include a mutation in at least one nucleotide of the wild type M5 sequence set forth by SEQ ID NO: 16 (CAATG), a mutation in at least one nucleotide position of the wild type M7 set forth by SEQ ID NO: 18 (ACTTT) and at least one copy of the wild type M8 sequence set forth by SEQ ID NO: 19 (GCTTC) are provided in SEQ ID NOs:345-348.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), a mutation in at least one nucleotide position of the wild type M7 set forth by SEQ ID NO: 18 (ACTTT) and at least one copy of the wild type M8 sequence set forth by SEQ ID NO: 19 (GCTTC) are provided in SEQ ID NOs:349-354.

Non-limiting examples of isolated polynucleotides which include a mutation in at least one nucleotide of the wild type M4 sequence set forth by SEQ ID NO: 15 (CATTC), a mutation in at least one nucleotide position of the wild type M6 set forth by SEQ ID NO: 17 (GGGTG), a mutation in at least one nucleotide position of the wild type M7 set forth by SEQ ID NO: 18 (ACTTT) and at least one copy of the wild type M8 sequence set forth by SEQ ID NO: 19 (GCTTC) are provided in SEQ ID NOs:355-361.

Non-limiting examples of isolated polynucleotides which include a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG), a mutation in at least one nucleotide position of the wild type M7 set forth by SEQ ID NO: 18 (ACTTT) and at least one copy of the wild type M8 sequence set forth by SEQ ID NO: 19 (GCTTC) are provided in SEQ ID NOs:362-365.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG), a mutation in at least one nucleotide position of the wild type M7 set forth by SEQ ID NO: 18 (ACTTT) and at least one copy of the wild type M8 sequence set forth by SEQ ID NO: 19 (GCTTC) are provided in SEQ ID NOs:366-369.

According to some embodiments of the invention, the isolated polynucleotide comprises at least one copy of the wild type M3 sequence set forth by SEQ ID NO: 21 (CTTTT) with additional wild type or mutated sequences derived from element X (SEQ ID NO:6).

Non-limiting examples of isolated polynucleotides which includes a mutation in at least one nucleotide of the wild type M4 sequence set forth by SEQ ID NO: 15 (CATTC) and at least one copy of the wild type M3 sequence set forth by SEQ ID NO: 21 (CTTTT) are provided in SEQ ID NOs:378-384.

Non-limiting examples of isolated polynucleotides which include a mutation in at least one nucleotide of the wild type M5 sequence set forth by SEQ ID NO: 16 (CAATG) and at least one copy of the wild type M3 sequence set forth by SEQ ID NO: 21 (CTTTT) are provided in SEQ ID NOs:628-634.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG) and at least one copy of the wild type M3 sequence set forth by SEQ ID NO: 21 (CTTTT) are provided in SEQ ID NOs:370-377.

Non-limiting examples of isolated polynucleotides which include a mutation in at least one nucleotide of the wild type M4 sequence set forth by SEQ ID NO: 15 (CATTC), at least one copy of the wild type M6 set forth by SEQ ID NO: 17 (GGGTG) and at least one copy of the wild type M3 sequence set forth by SEQ ID NO: 21 (CTTTT) are provided in SEQ ID NOs:385-390.

Non-limiting examples of isolated polynucleotides which include a mutation in at least one nucleotide of the wild type M5 sequence set forth by SEQ ID NO: 16 (CAATG), at least one copy of the wild type M6 set forth by SEQ ID NO: 17 (GGGTG) and at least one copy of the wild type M3 sequence set forth by SEQ ID NO: 21 (CTTTT) are provided in SEQ ID NOs:391-396.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M6 set forth by SEQ ID NO: 17 (GGGTG) and at least one copy of the wild type M3 sequence set forth by SEQ ID NO: 21 (CTTTT) are provided in SEQ ID NOs:397-401.

Non-limiting examples of isolated polynucleotides which include a mutation in at least one nucleotide of the wild type M4 sequence set forth by SEQ ID NO: 15 (CATTC), at least one copy of the wild type M7 sequence set forth by SEQ ID NO: 18 (ACTTT) and at least one copy of the wild type M3 sequence set forth by SEQ ID NO: 21 (CTTTT) are provided in SEQ ID NOs:402-409.

Non-limiting examples of isolated polynucleotides which include a mutation in at least one nucleotide of the wild type M5 sequence set forth by SEQ ID NO: 16 (CAATG), at least one copy of the wild type M7 sequence set forth by SEQ ID NO: 18 (ACTTT) and at least one copy of the wild type M3 sequence set forth by SEQ ID NO: 21 (CTTTT) are provided in SEQ ID NOs:410-417.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M7 sequence set forth by SEQ ID NO: 18 (ACTTT) and at least one copy of the wild type M3 sequence set forth by SEQ ID NO: 21 (CTTTT) are provided in SEQ ID NOs:418-423.

Non-limiting examples of isolated polynucleotides which include a mutation in at least one nucleotide of the wild type M4 sequence set forth by SEQ ID NO: 15 (CATTC), at least one copy of the wild type M6 set forth by SEQ ID NO: 17 (GGGTG), at least one copy of the wild type M7 sequence set forth by SEQ ID NO: 18 (ACTTT) and at least one copy of the wild type M3 sequence set forth by SEQ ID NO: 21 (CTTTT) are provided in SEQ ID NOs:424-425.

Non-limiting examples of isolated polynucleotides which include a mutation in at least one nucleotide of the wild type M5 sequence set forth by SEQ ID NO: 16 (CAATG), at least one copy of the wild type M6 set forth by SEQ ID NO: 17 (GGGTG), at least one copy of the wild type M7 sequence set forth by SEQ ID NO: 18 (ACTTT) and at least one copy of the wild type M3 sequence set forth by SEQ ID NO: 21 (CTTTT) are provided in SEQ ID NOs:538-540.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M6 set forth by SEQ ID NO: 17 (GGGTG), at least one copy of the wild type M7 sequence set forth by SEQ ID NO: 18 (ACTTT) and at least one copy of the wild type M3 sequence set forth by SEQ ID NO: 21 (CTTTT) are provided in SEQ ID NO:426.

Non-limiting examples of isolated polynucleotides which include a mutation in at least one nucleotide of the wild type M4 sequence set forth by SEQ ID NO: 15 (CATTC), a mutation in at least one nucleotide position of the wild type M6 set forth by SEQ ID NO: 17 (GGGTG) and at least one copy of the wild type M3 sequence set forth by SEQ ID NO: 21 (CTTTT) are provided in SEQ ID NOs:427-435.

Non-limiting examples of isolated polynucleotides which include a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG) and at least one copy of the wild type M3 sequence set forth by SEQ ID NO: 21 (CTTTT) are provided in SEQ ID NOs:436-444.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG) and at least one copy of the wild type M3 sequence set forth by SEQ ID NO: 21 (CTTTT) are provided in SEQ ID NOs:445-451.

Non-limiting examples of isolated polynucleotides which include a mutation in at least one nucleotide of the wild type M4 sequence set forth by SEQ ID NO: 15 (CATTC), a mutation in at least one nucleotide position of the wild type M7 set forth by SEQ ID NO: 18 (ACTTT) and at least one copy of the wild type M3 sequence set forth by SEQ ID NO: 21 (CTTTT) are provided in SEQ ID NOs:452-458.

Non-limiting examples of isolated polynucleotides which include a mutation in at least one nucleotide of the wild type M5 sequence set forth by SEQ ID NO: 16 (CAATG), a mutation in at least one nucleotide position of the wild type M7 set forth by SEQ ID NO: 18 (ACTTT) and at least one copy of the wild type M3 sequence set forth by SEQ ID NO: 21 (CTTTT) are provided in SEQ ID NOs:459-465.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), a mutation in at least one nucleotide position of the wild type M7 set forth by SEQ ID NO: 18 (ACTTT) and at least one copy of the wild type M3 sequence set forth by SEQ ID NO: 21 (CTTTT) are provided in SEQ ID NO:466.

Non-limiting examples of isolated polynucleotides which include a mutation in at least one nucleotide of the wild type M4 sequence set forth by SEQ ID NO: 15 (CATTC), a mutation in at least one nucleotide position of the wild type M6 set forth by SEQ ID NO: 17 (GGGTG), a mutation in at least one nucleotide position of the wild type M7 set forth by SEQ ID NO: 18 (ACTTT) and at least one copy of the wild type M3 sequence set forth by SEQ ID NO: 21 (CTTTT) are provided in SEQ ID NOs:467-471.

Non-limiting examples of isolated polynucleotides which include a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG), a mutation in at least one nucleotide position of the wild type M7 set forth by SEQ ID NO: 18 (ACTTT) and at least one copy of the wild type M3 sequence set forth by SEQ ID NO: 21 (CTTTT) are provided in SEQ ID NOs:472-477.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG), a mutation in at least one nucleotide position of the wild type M7 set forth by SEQ ID NO: 18 (ACTTT) and at least one copy of the wild type M3 sequence set forth by SEQ ID NO: 21 (CTTTT) are provided in SEQ ID NOs:478-483.

According to some embodiments of the invention, the isolated polynucleotide further comprises at least one copy of the wild type M8 sequence set forth by SEQ ID NO: 19 (GCTTC) and at least one copy of the wild type M3 sequence set forth by SEQ ID NO: 21 (CTTTT) with additional wild type or mutated sequences derived from element X (SEQ ID NO:6).

Non-limiting examples of isolated polynucleotides which includes a mutation in at least one nucleotide of the wild type M4 sequence set forth by SEQ ID NO: 15 (CATTC), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:484-495.

Non-limiting examples of isolated polynucleotides which include a mutation in at least one nucleotide of the wild type M5 sequence set forth by SEQ ID NO: 16 (CAATG), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:496-507.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:508-515.

Non-limiting examples of isolated polynucleotides which include a mutation in at least one nucleotide of the wild type M4 sequence set forth by SEQ ID NO: 15 (CATTC), at least one copy of the wild type M6 set forth by SEQ ID NO: 17 (GGGTG), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:516-519.

Non-limiting examples of isolated polynucleotides which include a mutation in at least one nucleotide of the wild type M5 sequence set forth by SEQ ID NO: 16 (CAATG), at least one copy of the wild type M6 set forth by SEQ ID NO: 17 (GGGTG), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:520-523.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M6 set forth by SEQ ID NO: 17 (GGGTG), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:524-525.

Non-limiting examples of isolated polynucleotides which include a mutation in at least one nucleotide of the wild type M4 sequence set forth by SEQ ID NO: 15 (CATTC), at least one copy of the wild type M7 sequence set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:526-529.

Non-limiting examples of isolated polynucleotides which include a mutation in at least one nucleotide of the wild type M5 sequence set forth by SEQ ID NO: 16 (CAATG), at least one copy of the wild type M7 sequence set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:530-533.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M7 sequence set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:534-535.

Non-limiting examples of isolated polynucleotides which include a mutation in at least one nucleotide of the wild type M4 sequence set forth by SEQ ID NO: 15 (CATTC), at least one copy of the wild type M6 set forth by SEQ ID NO: 17 (GGGTG), at least one copy of the wild type M7 sequence set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:536-537.

Non-limiting examples of isolated polynucleotides which include a mutation in at least one nucleotide of the wild type M5 sequence set forth by SEQ ID NO: 16 (CAATG), at least one copy of the wild type M6 set forth by SEQ ID NO: 17 (GGGTG), at least one copy of the wild type M7 sequence set forth by SEQ ID NO: 18 (ACTTT) at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:538-539.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), at least one copy of the wild type M6 set forth by SEQ ID NO: 17 (GGGTG), at least one copy of the wild type M7 sequence set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NO:540.

Non-limiting examples of isolated polynucleotides which include a mutation in at least one nucleotide of the wild type M4 sequence set forth by SEQ ID NO: 15 (CATTC), a mutation in at least one nucleotide position of the wild type M6 set forth by SEQ ID NO: 17 (GGGTG), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:541-547.

Non-limiting examples of isolated polynucleotides which include a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:548-554.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:555-559.

Non-limiting examples of isolated polynucleotides which include a mutation in at least one nucleotide of the wild type M4 sequence set forth by SEQ ID NO: 15 (CATTC), a mutation in at least one nucleotide position of the wild type M7 set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:560-566.

Non-limiting examples of isolated polynucleotides which include a mutation in at least one nucleotide of the wild type M5 sequence set forth by SEQ ID NO: 16 (CAATG), a mutation in at least one nucleotide position of the wild type M7 set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:567-573.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), a mutation in at least one nucleotide position of the wild type M7 set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:574-578.

Non-limiting examples of isolated polynucleotides which include a mutation in at least one nucleotide of the wild type M4 sequence set forth by SEQ ID NO: 15 (CATTC), a mutation in at least one nucleotide position of the wild type M6 set forth by SEQ ID NO: 17 (GGGTG), a mutation in at least one nucleotide position of the wild type M7 set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:579-583.

Non-limiting examples of isolated polynucleotides which include a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG), a mutation in at least one nucleotide position of the wild type M7 set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:584-588.

Non-limiting examples of isolated polynucleotides which include a mutation in at least one nucleotide of the wild type M4 sequence set forth by SEQ ID NO: 15 (CATTC), a mutation in at least one nucleotide of the wild type M5 sequence set forth by SEQ ID NO: 16 (CAATG), a mutation in at least one nucleotide position of the wild type M6 set forth by SEQ ID NO: 17 (GGGTG), a mutation in at least one nucleotide position of the wild type M7 set forth by SEQ ID NO: 18 (ACTTT), at least one copy of the wild type M8 sequence set forth by SEQ ID NO: 19 (GCTTC) and at least one copy of the wild type M3 sequence set forth by SEQ ID NO: 21 (CTTTT) are provided in SEQ ID NOs:589-592.

According to some embodiments of the invention, the isolated polynucleotide comprises at least one copy of wild type M3 sequence (SEQ ID NO: 21) and at least one copy of wild type M8 sequence (SEQ ID NO: 19), with at least one mutation in wild type M6 (SEQ ID NO: 17) and/or in wild type M7 (SEQ ID NO:50).

Non-limiting examples of isolated polynucleotides which include at least one copy of the wild type M8 sequence set forth by SEQ ID NO: 19 (GCTTC) and at least one copy of the wild type M3 sequence set forth by SEQ ID NO: 21 (CTTTT), with a mutation in at least one nucleotide of the wild type M6 sequence (SEQ ID NO: 17), and/or a mutation in at least one nucleotide of the wild type M7 (SEQ ID NO: 18) are provided in SEQ ID NOs:593-600.

The present inventors have envisaged that an isolated polynucleotide which includes the wild type M8 sequence (SEQ ID NO: 19) and/or the wild type M3 (SEQ ID NO: 21) sequence in addition to tissue specific enhancers (e.g., wild type M4 and/or wild type M5), and/or induced enhancers (e.g., developmentally related- or stress related-enhancers) is expected to exert a more specific regulatory effect by suppressing expression in non-target cells or under non-induced conditions.

According to some embodiments of the invention, the isolated polynucleotide comprises at least one copy of the wild type M8 sequence set forth by SEQ ID NO: 19 (GCTTC) and an endothelial specific enhancer sequence.

According to some embodiments of the invention, the isolated polynucleotide comprises at least one copy of the wild type M8 sequence set forth by SEQ ID NO: 19 (GCTTC) and at least one copy of wild type M4 sequence set forth by SEQ ID NO: 15.

According to some embodiments of the invention, the isolated polynucleotide comprises at least one copy of the wild type M8 sequence set forth by SEQ ID NO: 19 (GCTTC) and at least one copy of wild type M5 sequence set forth by SEQ ID NO:16.

According to some embodiments of the invention, the isolated polynucleotide comprises at least one copy of the wild type M8 sequence set forth by SEQ ID NO: 19 (GCTTC), at least one copy of wild type M4 sequence set forth by SEQ ID NO: 15 and at least one copy of wild type M5 sequence set forth by SEQ ID NO:16.

According to some embodiments of the invention, the isolated polynucleotide comprises at least one copy of the wild type M3 sequence set forth by SEQ ID NO: 21 (CTTTT) and an endothelial specific enhancer sequence.

According to some embodiments of the invention, the isolated polynucleotide comprises at least one copy of the wild type M3 sequence set forth by SEQ ID NO: 21 (CTTTT) and at least one copy of wild type M4 sequence set forth by SEQ ID NO: 15.

According to some embodiments of the invention, the isolated polynucleotide comprises at least one copy of the wild type M3 sequence set forth by SEQ ID NO: 21 (CTTTT) and at least one copy of wild type M5 sequence set forth by SEQ ID NO:16.

According to some embodiments of the invention, the isolated polynucleotide comprises at least one copy of the wild type M3 sequence set forth by SEQ ID NO: 21 (CTTTT), at least one copy of wild type M4 sequence set forth by SEQ ID NO: 15 and at least one copy of wild type M5 sequence set forth by SEQ ID NO:16.

According to some embodiments of the invention, the isolated polynucleotide comprises at least one copy of the wild type M3 sequence set forth by SEQ ID NO: 21 (CTTTT), at least one copy of wild type M8 sequence set forth by SEQ ID NO: 19 (GCTTC) and an endothelial specific enhancer sequence.

According to some embodiments of the invention, the isolated polynucleotide comprises at least one copy of the wild type M3 sequence set forth by SEQ ID NO: 21 (CTTTT), at least one copy of wild type M8 sequence set forth by SEQ ID NO: 19 (GCTTC) and at least one copy of wild type M4 sequence set forth by SEQ ID NO: 15.

According to some embodiments of the invention, the isolated polynucleotide comprises at least one copy of the wild type M3 sequence set forth by SEQ ID NO: 21 (CTTTT), at least one copy of wild type M8 sequence set forth by SEQ ID NO: 19 (GCTTC) and at least one copy of wild type M5 sequence set forth by SEQ ID NO: 16.

According to some embodiments of the invention, the isolated polynucleotide comprises at least one copy of the wild type M3 sequence set forth by SEQ ID NO: 21 (CTTTT), at least one copy of wild type M8 sequence set forth by SEQ ID NO: 19 (GCTTC), at least one copy of wild type M4 sequence set forth by SEQ ID NO: 15 and at least one copy of wild type M5 sequence set forth by SEQ ID NO: 16.

According to some embodiments of the invention, the isolated polynucleotide comprises at least one copy of the wild type M3 sequence set forth by SEQ ID NO: 21 (CTTTT), at least one copy of wild type M8 sequence set forth by SEQ ID NO: 19 (GCTTC) and at least one enhancer element such as wild type M6 (SEQ ID NO: 17) and/or wild type M7 sequence (SEQ ID NO:18).

According to some embodiments of the invention, the isolated polynucleotide includes at least one copy of wild type M8 with additional flanking sequences such as at least one copy of a wild type M8 sequence (SEQ ID NO:19), at least one copy of wild type M7 (SEQ ID NO: 18) and/or wild type M9 sequence (SEQ ID NO: 14, CTGGA); and/or the isolated polynucleotide includes at least one copy of wild type M8 and at least one mutation in M7, with or without M9 (SEQ ID NO: 22). Such polynucleotides can be used as a non-specific repressor.

According to some embodiments of the invention, the isolated polynucleotide is for increasing expression of a heterologous polynucleotide operably linked thereto in cells/tissues.

According to some embodiments of the invention, the isolated polynucleotide comprises at least one copy of wild type M6 sequence set forth by SEQ ID NO: 17 (GGGTG) and/or at least one copy of wild type M7 sequence set forth by SEQ ID NO: 18 (ACTTT).

According to some embodiments of the invention, the isolated polynucleotide includes at least one copy of wild type M6 (SEQ ID NO: 17) and a mutation in at least one nucleotide of wild type M8 (SEQ ID NO: 19).

Non-limiting examples of isolated polynucleotide which include at least one copy of wild type M6 (SEQ ID NO: 17) and a mutation in at least one nucleotide of the wild type M8 (SEQ ID NO: 19) are provided in SEQ ID NOs:23-26.

According to some embodiments of the invention, the isolated polynucleotide includes at least one copy of wild type M7 (SEQ ID NO: 18) and a mutation in at least one nucleotide of wild type M8 (SEQ ID NO: 19).

Non-limiting examples of isolated polynucleotide which include at least one copy of wild type M7 (SEQ ID NO: 18) and a mutation in at least one nucleotide of the wild type M8 (SEQ ID NO: 19) are provided in SEQ ID NOs:27-28.

According to some embodiments of the invention, the isolated polynucleotide includes at least one copy of wild type M6 (SEQ ID NO: 17), at least one copy of wild type M7 (SEQ ID NO: 18) and a mutation in at least one nucleotide of wild type M8 (SEQ ID NO: 19).

According to some embodiments of the invention, the isolated polynucleotide includes at least one copy of wild type M1 (SEQ ID NO: 20) and a mutation in at least one nucleotide of wild type M8 (SEQ ID NO: 19).

Non-limiting examples of isolated polynucleotide which include at least one copy of wild type M1 (SEQ ID NO: 20) and a mutation in at least one nucleotide of the wild type M8 (SEQ ID NO: 19) are provided in SEQ ID NOs:43-54 and 601-632.

According to some embodiments of the invention, the isolated polynucleotide includes at least one copy of wild type M1 (SEQ ID NO: 20), at least one copy of wild type M6 (SEQ ID NO: 17) and/or at least one copy of wild type M7 (SEQ ID NO: 18) and a mutation in at least one nucleotide of wild type M8 (SEQ ID NO: 19).

Non-limiting examples of isolated polynucleotides which include a mutation in at least one nucleotide of wild type M8 (SEQ ID NO: 19) and at least one copy of wild type M1 (SEQ ID NO: 20), wild type M6 (SEQ ID NO: 17) and/or wild type M7 (SEQ ID NO: 18) are provided in SEQ ID NOs:29-42.

Additional examples of regulatory isolated polynucleotides which can be used according to some embodiments of the invention are provided (; SEQ ID NOs: 633-644) in the Examples section which follows.

According to an aspect of some embodiments of the invention, there is provided an isolated polynucleotide comprising a nucleic acid sequence which comprises a first polynucleotide comprising the pre-proendothelin (PPE-1) promoter set forth by SEQ ID NO:13 and a second polynucleotide comprising at least one copy of a nucleic acid sequence selected from the group consisting of:

| (i) | wild type M4 sequence set forth by SEQ ID NO: 15 (CATTC), |
|---|---|
| (ii) | wild type M5 sequence set forth by SEQ ID NO: 16 (CAATG), |
| (iii) | wild type M8 sequence set forth by SEQ ID NO: 19 (GCTTC), |
| (iv) | wild type M6 sequence set forth by SEQ ID NO: 17 (GGGTG), |
| (v) | wild type M7 sequence set forth by SEQ ID NO: 18 (ACTTT); |
| (vi) | wild type M1 sequence set forth by SEQ ID NO: 20 (GTACT), |
| and | |
| (vii) | wild type M3 sequence set forth by SEQ ID NO: 21 (CTTTT); |

with the proviso that the second polynucleotide is not SEQ ID NO:6 (element X), and wherein the isolated polynucleotide is not SEQ ID NO:12 (PPE-1-3X).

According to some embodiments of the invention, each of the wild type M4, M5, M8, M6, M7 and/or M1 sequences is placed in a head to tail (5→3′) orientation with respect to the PPE-1 promoter set forth by SEQ ID NO:13.

According to some embodiments of the invention, each of the wild type M4, M5, M8, M6, M7 and/or M1 sequences is placed in a tail to head (3′→5′) orientation with respect to the PPE-1 promoter set forth by SEQ ID NO:13.

According to some embodiments of the invention, the wild type M4, M5, M8, M6, M7 and/or M1 sequences are placed in various orientations (head to tail or tail to head) and/or sequential order with respect the other wild type M4, M5, M8, M6, M7 and/or M1 sequences, and/or with respect to the orientation of SEQ ID NO:13.

Construction of such viral vectors may be effected using known molecular biology techniques such as those described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986].

Construction of the virus of SEQ ID NO: 9 is described in International Application WO/2008/132729, the contents of which are incorporated herein by reference. Construction of the Ad5-PPE-1-3X-Fas-c vector is described in great details in Example 2 of the Examples section, which follows.

The non-replicating adeno-virus of the invention is introduced into PER.C6® cells, available from Crucell™ (wwwdotcrucelldotcom). Example 3 below, describes an exemplary protocol of cell infection using a transfection reagent, Lipofectamine™ (Invitrogen).

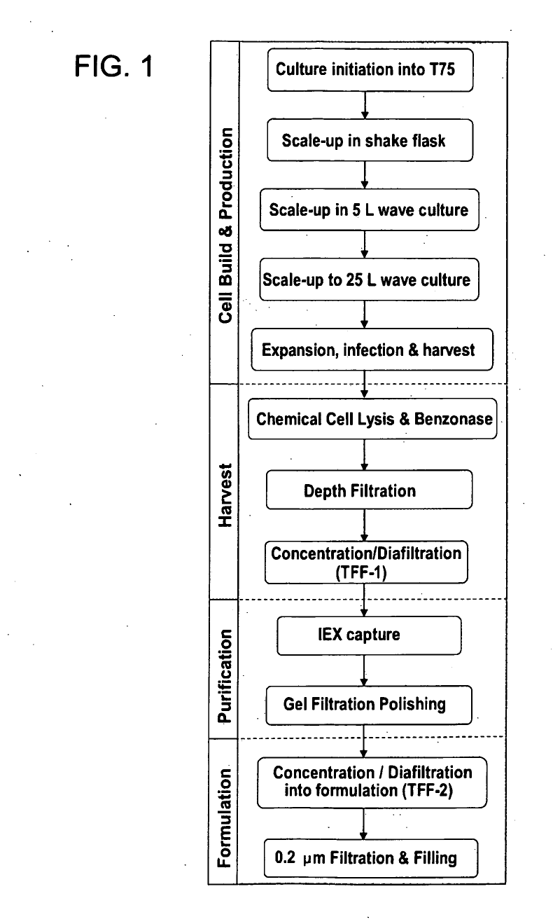

An outline of the key steps in a 25 L manufacturing process is shown in FIG. 1. This process is based on the initial cell culture being performed in a disposable 50 L Wave type reactor, followed by cell lysis and clarification and buffer exchange operations performed with disposable membrane technologies, followed by a two step chromatography purification process, and a final concentration and formulation operation again performed using disposable technologies. Scale-down models exist for the individual operations and for development purposes. As the process is based on scalable operations the manufacturing scale can also be increased from the planned manufacturing scale of 25 L without significant process changes.

It should be noted that the above description is only exemplary and by no means is intended to limit the scope of the invention.

Thus, as mentioned the cells are grown in suspension to increase viral yield.

As used herein a “suspension culture” refers to a culture in which cells multiply, while suspended in a suitable medium (as opposed to an adherent culture in which cells adhere to the culture vessel). Culturing is effected in a disposable or non-disposable bioractor.

Briefly, according to a specific embodiment the culture is initiated in small flasks (e.g., 75 cm²). A multistep process may be undertaken for reaching the final culture medium. For instance, through a 5 L to 25 L leap. Thus, the culture is initiated in a 10 L culture (e.g., wave culture) and increased to 25 L. Culturing is preferably effected in disposable dishes/bags, as described in the Examples section which follows, such as using the Wave reactor system (e.g., Wave 50-200 L) or Stri-Tank, hyclone SUB250-500 L.

According to a specific embodiment, culturing is effected at a 5-200 L volume culture.

According to a specific embodiment, culturing is effected at a 50-200 L volume culture.

According to a specific embodiment, culturing is effected at a 50-100 L volume culture.

According to a specific embodiment, culturing is effected at a 5-100 L volume culture.

According to a specific embodiment, culturing is effected at a 5-50 L volume.

According to a specific embodiment, culturing is effected at a 5-25 L volume.

According to a specific embodiment, culturing is effected at a 25 L volume.

According to a specific embodiment, culturing is effected at a 50 L volume.

The culture is expanded while exhibiting varying values of MOI an optimal value of same is selected as the point of recovery.

Within the following paragraphs the individual stages post culturing are reviewed.

Thus, the instant invention further comprises recovering the non-replicating adenovirus vector from the cells following said culturing.

According to a specific embodiment, recovering is effected at a point of harvest (POH) of 3-4 days post infection and an MOI of 5.

In order to recover the virus, the cells are subjected to lysis. According to a specific embodiment, recovering is effected by subjecting said cells to a detergent lysis.

Detergent-based cell lysis is an alternative to physical disruption of cell membranes, although it is sometimes used in conjunction with homogenization and mechanical grinding. Detergents disrupt the lipid barrier surrounding cells by disrupting lipid:lipid, lipid:protein and protein:protein interactions. The ideal detergent for cell lysis depends on cell type and source and on the downstream applications following cell lysis. In general, nonionic and zwitterionic detergents are milder, resulting in less protein denaturation upon cell lysis, than ionic detergents and are used to disrupt cells when it is critical to maintain protein function or interactions. CHAPS, a zwitterionic detergent, and the Triton X series of nonionic detergents are commonly used for these purposes. In contrast, ionic detergents are strong solubilizing agents and tend to denature proteins, thereby destroying protein activity and function. SDS, an ionic detergent that binds to and denatures proteins, is used extensively for studies assessing protein levels by gel electrophoresis and western blotting. In addition to the choice of detergent, other important considerations for optimal cell lysis include the buffer, pH, ionic strength and temperature. Specific conditions for detergent-based lysis are provided in the Examples section.

Once the cells are lysed, cellular DNA and cell debris are removed so as to obtain a clear feedstock. The clear feedstock is subjected to TFF (see Example 8) so as to obtain a concentrated viral pellet.

This concentrated pellet is now subject to further purifications. According to a specific embodiment, the purification is effected by subjecting the viral pellet to anion exchange chromatography and size exclusion chromatography (e.g., IEX capture and Gel filtration polishing of the “purification” step in FIG. 1). The purified batch is formulated and filtrated.

According to a specific embodiment, sterile filtration is done using a 0.2 μm PES sterile mini Capsule filter and filling of 1.1 ml aliquots into 1.8 ml cryovials.

According to another specific embodiment, the final product is stored in copolymer vials) Topas®, an advanced cyclolefin polymer) 2-5 ml with stopper. According to yet another embodiment, the final product is stored in Glass vials such as those available from West Pharmaceuticals (3-5 ml with stopper).

The final product is stored at ≦−65° C.

The final formulated batches (as well as any step preceding same) are subject to various quality control assays, such as described hereinbelow.

Harvests conforming to in-process specifications (Microbial limit, Mycoplasma) may be pooled.

Tables 4-5 below, illustrate non-limiting analytical assays for providing product characterization, in process, release and stability testing.

TABLE 4
methods used for batch release

| Fraction | |
|---|---|
| Tested | Method |
| Harvest | Identity by PCR |
| | Mycoplasma |
| | Bioburden |
| | In vitro Test for Detection of viral Contaminants in Adeno |
| | viral material using MRC-5, Vero & HeLa detector cell lines |
| | In vivo test for presence of in apparent viruses using suckling |
| | mice, adult mice, guinea pigs & embryonated eggs |
| Bulk Drug | RCA (Detection of replication competent Adenovirus using |
| Substance | the A549 detector cell line) |
| | Host Cell DNA Residues (qPCR) |
| | ELISA for detection of PER.C6 Host Cell Protein |
| | Residual Triton by Reverse Phase HPLC |
| | Residual Benzonase |
| Final | Appearance |
| Product | pH |
| | Quantitation of Viral Particles by OD₂₆₀ |
| | Potency by Plaque Forming Unit Assay (PFU)) |
| | Western Blot Analysis of Transgene Expression |
| | Sterility |
| | Endotoxins |
| | General Safety Test |

* Some of the tests performed on the Bulk Drug Substance (BDS) fraction
may also performed on the final product.

TABLE 5
Methods Used for In Process Testing

| Fraction/Stage | |
|---|---|
| Tested | Method |
| Harvest | Cell Count |
| | Bioburden |
| | Analysis & Determination of Titer using AEX-HPLC |
| | Infectious Titre of Adenovirus by Immunocytochemistry/or |
| | Potency by Plaque Forming Unit Assay (PFU) |
| Down Stream | Cell Count |
| Process | Analysis & Determination of Titer using AEX-HPLC |
| (in all stages) | Infectious Titre of Adenovirus by Immunocytochemistry |
| Bulk Drug | Bioburden |
| Substance | pH |
| | Appearance |
| | Protein Concentration by Bradford |
| | SDS-PAGE for Purity/Identity |
| | Analysis & Determination of Titer using AEX-HPLC |
| | Infectious Titre of Adenovirus by Immunocytochemistry/or |
| | Quantitation of Viral Particles by OD₂₆₀ |

Appearance

This test pertains to the final product that has been frozen and thawed. The final product is white or colorless.

Identity by PCR (In Process Control (IPC) Test Only)

The assay includes pAC-PPE-1-3X-Fas-C DNA as positive control and specific primers [PPE CTC TTG ATT CTT GAA CTC TG (SEQ ID NO: 645) and p55 TAC AAG TAG GTT CCT TTG TG (SEQ ID NO: 646)], yielding a DNA segment of about 750 bp including part of the PPE-1-3X promoter and part of the TNF-R1. This segment is unique to the final product and is therefore used for positive identification of the final product. The resulting DNA is analyzed on an agarose gel in comparison with the positive control.

Mycoplasma

This is an in-process test performed on the viral harvest, complying with the European Pharmacopoeia, section 2.6.7.

Both an indicator DNA fluorochrome test and a cultivation assay are performed. Test sensitivity is sufficient to detect >100 cfu/ml.

Mycoplasma (PCR)

EZ-PCR Mycoplasma Test Kit (Biological Industries, 20-700) is used to detect possible contamination with mycoplasma. The sample, a positive mycoplasma control, and a negative control sample (no DNA) all undergo PCR with primers designed to amplify mycoplasma DNA. The PCR products undergo electrophoresis on a 1% agarose gel and the resulting bands are compared visually.

Bioburden (Microbial Limit Test)

This is an in-process test performed on the viral harvest, complying with the European Pharmacopoeia section 2.6.12, Microbial Examination of Non-Sterile Products (Total Viable Aerobic Count).

Test sensitivity is sufficient to detect approximately 100 cfu/ml.

In Vitro Test for Detection of Viral Contaminants in Adenoviral Material Using MRC-5, VERO & HeLa Detector Cell Lines (Detection of Viral ADA—Adventitious Agents)

The test article is neutralized with anti-Adenovirus type 5 antibodies and is then used to inoculate cultures of MRC-5, Vero, and HeLa detector cell lines. All cultures are observed for evidence of cytopathic effect (CPE). On day 14 post inoculation a sub-culture is performed on all cultures not displaying CPE. The sub-cultures are maintained for an additional 14 days and are observed for CPE. At the end of the culture period the cultures are tested for the ability to haemadsorb a mixture of red blood cells from various species, as a sign of viral contamination. Samples of the test article are spiked and cultured as controls. Test sensitivity is 100 TCID50/ml.

In Vivo Test for Presence of Inapparent Viruses Using Suckling Mice, Adult Mice, Guinea Pigs & Embryonated Eggs (Chicken)

The test is performed according to FDA “Points to consider in Characterization of Cell Lines Used to Produce Biologicals (1993)”

Detection of Replication Competent Adenovirus (RCA)

In this assay, the presence of RCA in 3×10¹⁰ vp of the virus is detected by inoculation onto the human lung carcinoma cell line A549. Assays are performed to establish a suitable inoculum level at which, there is no interference and no cytotoxicity that is not related to RCA. Low levels of Adenovirus are amplified by three passages of the cultures with observation for evidence of cytopathic effect at each passage. Test sensitivity is 10-100 TCID₅₀.

Host Cell DNA Residues

Real time PCR is used to detect and quantify the Adenovirus E1 gene. This gene exists in the PER.C6 host cells and is essential for virus propagation, but has been deleted from the final product. If the gene is not detected, absence of host cell DNA is inferred. Assay sensitivity is 78.13 pg/ml, based on testing 8 μl of nucleic acid extracted from neat sample.

ELISA for Detection of PER.C6 Host Cell Protein

An Elisa method is used for detection of residual Host Cell Protein (HCP) in VB-111 Bulk Drug Substance or Drug Product. An Elisa kit which captures Per.C6 HCPs is used for the assay. Samples and standards are incubated with primary (coated on microtiter strips) and secondary antibodies in microtiter wells, then a substrate is added to yield a colorimetric change. Comparison of samples to a standard curve enables quantification of Residual HCP in the VB-111 sample.

Cs Residues

The sample is digested in a solution of 2% Nitric Acid in Purified Water and is then analyzed by ICP (Inductivity Coupled Plasma) Mass Spectrometry. The sample solution is introduced by pneumatic nebulization into radio frequency plasma where energy transfer processes cause desolvation, atomization, and ionization. The ions are extracted from the plasma through a differentially pumped vacuum interface and separated on the basis of their mass-to-charge ratio by a quadruple mass spectrometer. This test has a quantitation limit of 0.1 μg/ml.

Residual Triton by Reverse Phase HPLC

Triton X-100 is used for cell lysis as part of the manufacturing process of the virus.

This procedure determines the residual Triton X-100 in VB-111 samples by RP-HPLC in VB-111 Bulk Drug Substance or Drug Product.

Residual Benzonase

Benzonase endonuclease is used to reduce cell DNA levels. ELISA method is used to determine residual levels of Benzonase.

Elisa kit (Merck) includes polyclonal antibodies specific to Benzonase in pre-coated wells of polystyrene microtiter plates to which samples are added. Horse Radish Peroxidase (HRP) conjugated anti-benzonase antibodies are then added, and TMB (Tetramethylbenzidine, Hydrogen Peroxide) is used to visualize the bound sandwich complexes. The reaction is stopped by adding 0.2M H₂SO₄. The plate is read at 450 nm by a microtiter plate reader.

Protein Concentration by Bradford (IPC Test)

This method is used to determine the loading volume of solubilized protein concentration of VB-111 purified samples. BSA standards and a reference are run alongside the test sample and the results are compared.

SDS-PAGE for Purity/Identity (IPC Test Only)

This method provides visualization of presence of viral proteins when compared with a reference standard using an SDS-PAGE gel which is then stained with Colloidal Blue.

Analysis and Determination of Titer for Adenovirus Samples Using AEX-HPLC (IPC Test Only)

This method is used along the purification process from Harvest to BDS. titer determination using HPLC analysis is performed along the purification process. The method uses a salt gradient on an anion exchange phase HPLC column

Infectious Titer of Adenovirus by ImmunoCytoChemical Assay (IPC Test Only)

ImmunoCytoChemical (ICC) assay is used in-process to determine adenovirus infectious titer. This method utilizes an antibody against human adenovirus hexon capsid protein. Infectious titer is obtained in 3-days.

pH

According to USP <791>

Quantitation of Virus Particles

The determination of vp/ml is based on quantification of viral DNA by its optical density at A260 (1 OD₂₆₀ unit is equivalent to 1.1×10¹² viral particles, Green and Pina, 1963). In preparation for this test an SDS solution is added to the viral sample; the SDS dissolves the viral protein coat and the DNA is released. OD is read in the range 0.05-1: ARM—Adenovirus Reference Material Human, Adenovirus 5 reference, ATCC Cat #VR-1516, used as a reference in this assay was found to give results within the range recommended by the FDA.

Potency by Plaque Forming Units Assay

The PFU assay is based on serial dilutions of the vector that are added to sub-confluent cultures of HEK293 cells, overlaid with agarose, incubated at 37° C., and are followed for plaque formation. The plaques are counted at the end of the incubation period and the value of PFU per ml of the viral suspension is then calculated.

ARM—Adenovirus Reference Material Human, Adenovirus 5 reference, ATCC Cat #VR-1516, used as a reference in this assay was found to give results within the range recommended by the FDA.

Western Blot Analysis of Transgene Expression

The expression level of the transgene is quantified using an anti human TNF-Receptor antibody in a western blot analysis. The Fas chimera transgene includes domains of the human TNFR1 (Tumor Necrosis Factor Receptor 1), and can therefore be used in this assay as an indicator protein for the quantitation of the trannsgene expression level in endothelial cell culture. The level of the expressed protein is determined visually by comparing the intensity of the TNFR band in the sample to the various loads of the TNF-R1 used as a calibrator standard (2-12 ng/ml), analyzed on a 10% Bis-Tris gel followed by western blotting using h-TNF-R1 antibodies

Sterility

According to PhEur, JP, & USP, harmonized version.

Endotoxins Chromogenic Assay

According to USP <85>

General Safety

According to Food and Drugs Part 610.11 General Safety (2004).

Detection of Adeno Associated Virus (AAV)

This test is performed by real time PCR. As amplification of the target molecule proceeds, a reporter dye is released from the 5′ end of the probe and fluorescence increases in proportion to the increase in the PCR product. Detection limit is 10¹ DNA copies (performed on the MVB and on the early batches).

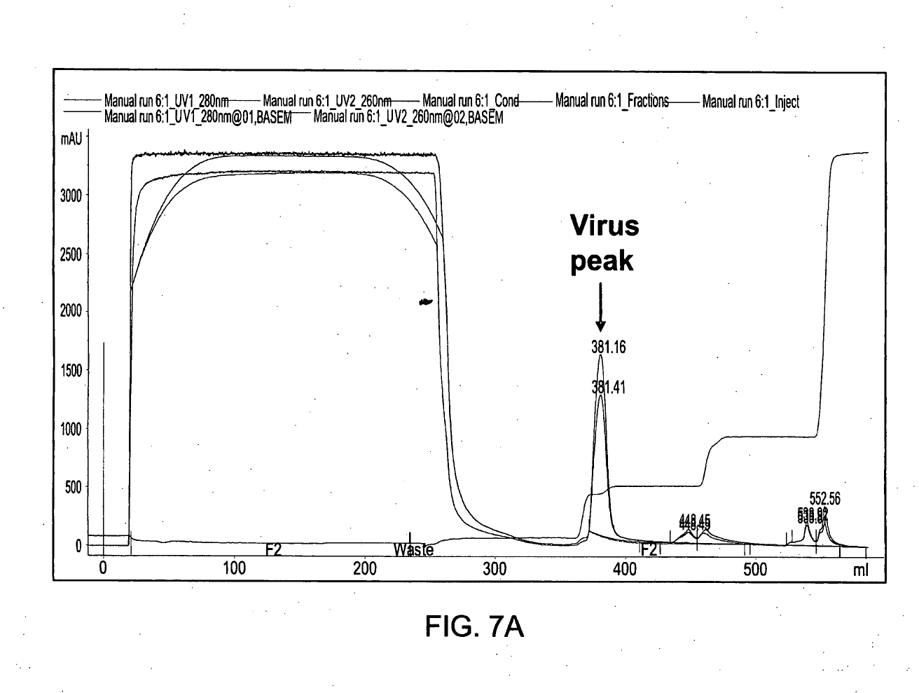

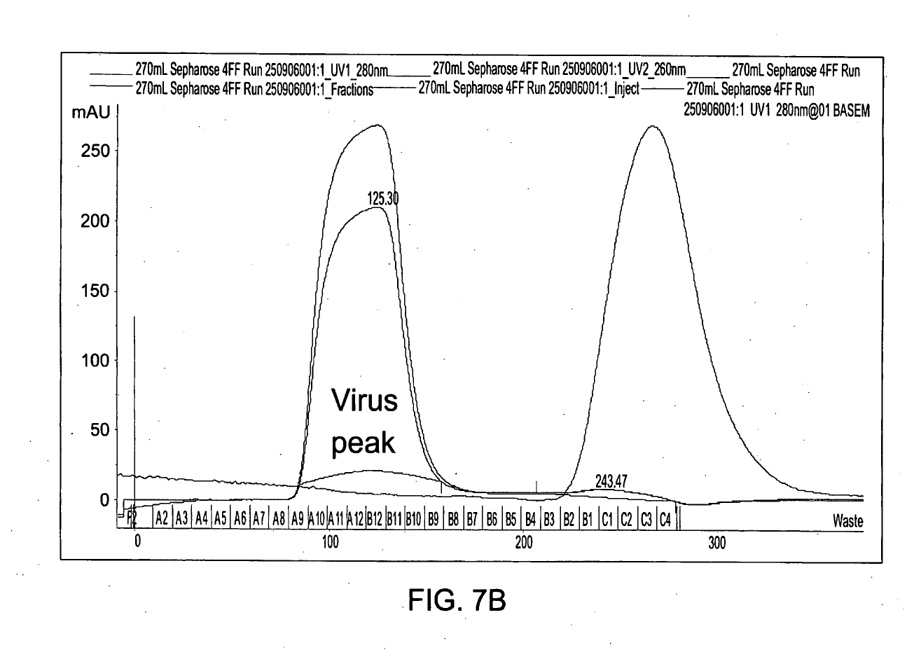

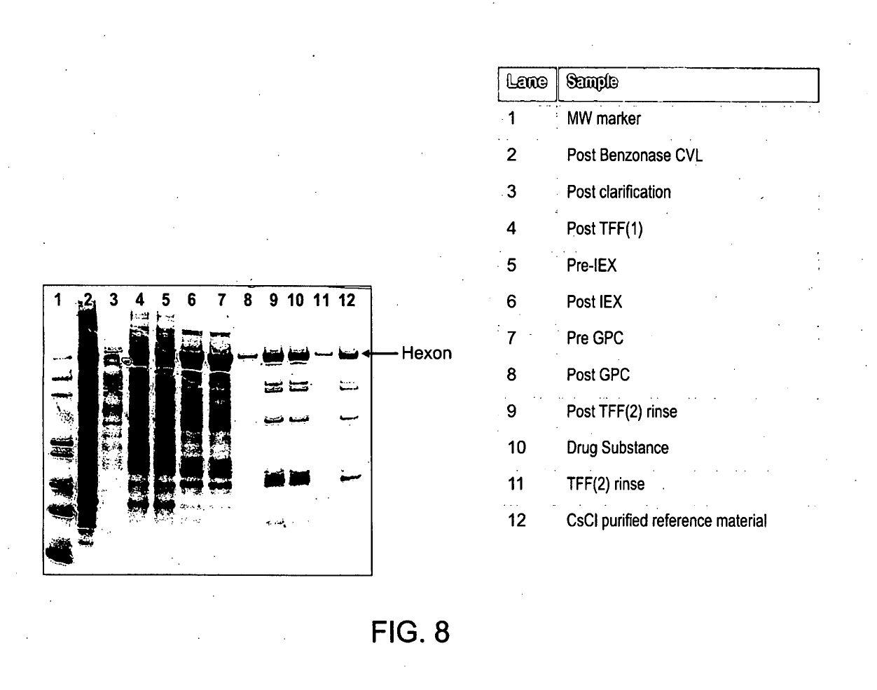

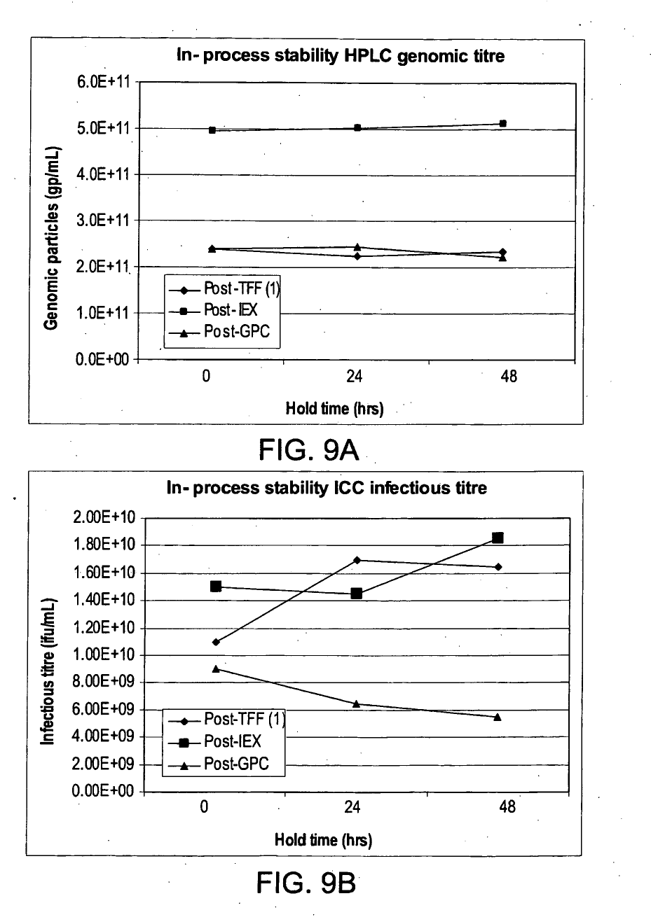

The final preparation (e.g., generated according to the above described large scale process) is characterized by ion exchange and size exclusion chromatography traces of FIGS. 7A-B and product profile of Table 6, below.

TABLE 6
Product specifications
(manufactured in non-adherent cells grown in serum free medium)

| Fraction | Parameter | Specifications | |
|---|---|---|---|
| Harvest | Identity | Co-migration in gel with positive control | |
| | PCR | | |
| | Impurities | Negative | |
| | Mycoplasma, indicator DNA fluorochrome | | |
| | test and cultivation assay | | |
| | Bioburden | ≦10 CFU/ml | |
| | In vitro Test for detection of viral | Negative | |
| | contaminants in Adenoviral material using | | |
| | MRC-5, VERO & HeLa detector cell lines | | |
| | In vivo test for presence of inapparent viruses | Negative | |
| | using suckling mice, adult mice, guinea pigs | | |
| | & embryonated eggs | | |
| Bulk Drug | Impurities | <1 RCA/3 × 10¹⁰ VP | |
| Substance (BDS)* | RCA (Detection of replication competent | | |
| | Adenovirus using the A549 detector cell line) | | |
| | Residual DNA qPCR | <5 ng/ML | |
| | | Result: 0.78 | |
| | | Report Result | |
| | ELISA for Detection of PER.C6 Host Cell | <5000 ng/ML | |
| | Protein | RESULTs: 1260 ng/ml | |
| | | .706 ng/ml | |
| | | Report Result | |
| | Residual Triton by Reverse Phase HPLC | 0-50 PPM | |
| | | Results: 0 ppm | |
| | | Report Result | |
| | Residual Benzonase | <5 NG/ML | |
| | | Results: 0.1 ng/ml | |
| | | Report Result | |
| Final Product | Appearance | White or colorless | |
| | Quantitation | ≧0.80 × 10¹² VP/m1 | |
| | Viral particles, OD₂₆₀ | | |
| | Potency | ≧3 × 10¹⁰ PFU/ml | |
| | PFU (Plaque Forming Units) | | |
| | Transgene expression, Western Blot | Positive | |
| | VP/PFU ratio | ≦30 | |
| | Impurities | Negative | |
| | Sterility | | |
| | Endotoxins, Chromogenic assay | ≦200 EU/dose | |
| | General Safety Test | 1) | The animals survive the test period |
| | | 2) | The animals do not exhibit any response |
| | | | which is not specific for or expected from |
| | | | the product and which may indicate a |
| | | | difference in its quality |
| | | 3) | The animals weight no less at the end of |
| | | | the test period than at the time of injection |

*Testing may be performed on BDS or alternatively on the Final Product

According to a specific embodiment, the viral preparation may comprise a detergent (e.g., Triton X-100).

According to a specific embodiment, the traces of detergent (e.g., Triton X-100) are in the range of, 10-100, 50-100 ppm or according to a specific embodiment 0-100 ppm, as assayed by HPLC.

According to a further specific embodiment, the detergent concentration is zero, as determined by HPLC.

Thus, the present invention also contemplates a pharmaceutical composition comprising as an active ingredient the above-described viral preparation (e.g., using the large-scale production method).

The purpose of a pharmaceutical composition is to facilitate administration of the active ingredient to an organism.

As used herein a “pharmaceutical composition” refers to a preparation of one or more of the active ingredients described herein (i.e., viral vector) with other chemical components such as physiologically suitable carriers and excipients.

Herein the term “active ingredient” refers to the viral vector of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. According to a specific embodiment, the formulation comprises PBS with 10% glycerol which prior to administration (e.g., by i.v. injection) is diluted with saline (according to a specific embodiment the dilution factor is 1/5, e.g., 1 ml of drug and 4 ml saline.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, intradermal, intraperitoneal, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections, sublingual, rectal, transdermal, intranasal, vaginal and inhalation routes. Injection of the viral vectors into a spinal fluid can also be used as a mode of administration.

The viral vectors or compositions thereof can be administered in an in-patient or out-patient setting. In one particular embodiment, the viral vectors or compositions thereof are administered in an injection or in an intravenous drip.

The present invention also contemplates engineering of the viral vectors in order to avoid, suppress or manipulate the immune response, ideally resulting in sustained expression and immune tolerance to the transgene product—such methods are described for example in Nayak et al., Gene Therapy (12 Nov. 2009), incoporated herein by reference.

Alternately, one may administer the pharmaceutical composition in a local rather than systemic manner, for example, via injection of the pharmaceutical composition directly into the tissue or tumor mass of a patient and even more directly into the tumor cells themselves.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (i.e. viral particles) effective to prevent, alleviate or ameliorate symptoms of a disorder (e.g., thyroid cancer, neuroendocrine cancer) or prolong the survival of the subject being treated.

Determination of a therapeutically effective amount is well within the capability of those skilled in the art, especially in light of the detailed disclosure provided herein. Therapeutic efficacy of administration of the adenoviral vector of the present invention can be assessed according to a variety of criteria, including clinical presentation, biochemical parameters, radiological evaluation and the like. In some embodiments, efficacy is evaluated according to one or more of the following exemplary parameters:

Biodistribution: for example, levels of virus DNA in blood and urine samples, expression of the fas-c transgene (mRNA) in blood;

Antibodies: for example, levels of total anti-Ad-51 g, IgG and neutralizing anti-Ad5 antibodies in serum;

Angiogenic biomarkers: for example, von Willebrand Factor and TNFα levels in the blood;

Cytokine levels: for example, peripheral blood cytokine levels;

Tumor response: Tumor dimensions can be measured on CT (or MRI) scans; or other radiographic means. Tumor response can then be evaluated according to accepted criteria, such as Response Evaluation Criteria in Solid Tumors (RECIST).

The criteria can be evaluated at any time following administration, and can also be compared to pre-dosing values. In one embodiment, the evaluation criteria are assessed prior to administration of the adenovirus vector, and then on days 4±1, 7±1, 14±1, 28±2, day 56±3, day 112±4, about 3 months, about 4 months, about 5 months, about 6 months, about 1 year or more post dosing.

Determination of safety of dosing or dosing regimen is well within the ability of one skilled in the art. Safety can be assessed according to a variety of criteria, including, but not limited to, clinical presentation, tissue and organ pathology, presence of abnormal vital signs (e.g. pyrexia, fatigue, chills, tachycardia, hypertension, constipation and the like), hematology values (e.g. hemoglobin, hematocrit, RCV and the like), chemistry or urinalysis abnormalities (elevated enzymes such as alkaline phosphatase ALT, AST, bilirubin and the like) and ECG, EEG, etc.

The therapeutically effective amount of the active ingredient can be formulated in a unit dose. As used herein “unit dose” refers to a physically discrete unit containing a predetermined quantity of an active material calculated to individually or collectively produce a desired effect such as an anti-cancer effect. A single unit dose or a plurality of unit doses can be used to provide the desired effect, such as an anti-cancer therapeutic effect.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The pharmaceutical compositions of the invention can be used to treat diseases or conditions associated with aberrant angiogenesis alone or in combination with one or more other established or experimental therapeutic regimen for such disorders (e.g., cancer and even more specifically primary or metastatic solid tumor). Therapeutic regimen for treatment of cancer suitable for combination with the nucleic acid constructs of the present invention or polynucleotide encoding same include, but are not limited to chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

All the information contained therein is incorporated herein by reference.

### Example 1

### Generation of Adherent PER.C6 WCB (Working Cell Bank)

The working cell bank (WCB) was propagated under GMP conditions to create the VBL WCB WCBP6001. A vial of the Crucell WCB (Lot #B127-006, p36), was thawed and expanded through serial passages to P(passage)39. These cells were harvested at 70% confluence and stored as a working cell bank in 1 ml aliquots in liquid N2. The cells are of human origin, viable, negative for bacteria and fungi, negative for mycoplasma, no exhibition of CPE, No HA, No HAD, as determined by in vitro assay for Adventitious viruses, negative for in apparent viruses (using suckling mice, adult mice, guinea pigs and embryonated eggs).

### Example 2

### Generation of Suspended (Non Adherent) PER.C6 MCB (Master Cell Bank)

Whilst it is possible to produce ppe-1-3x-Fasc using adherent cell culture approach, it is preferable to use suspension cell cultures where possible, due to the scale limitation of adherent cell cultures and the need for serum-containing media.

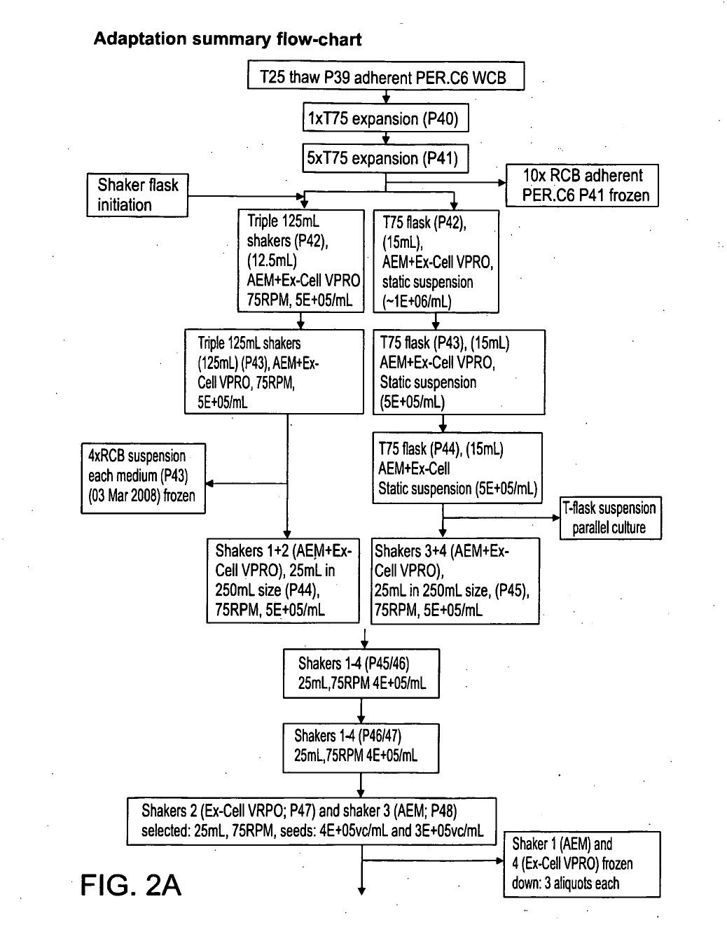

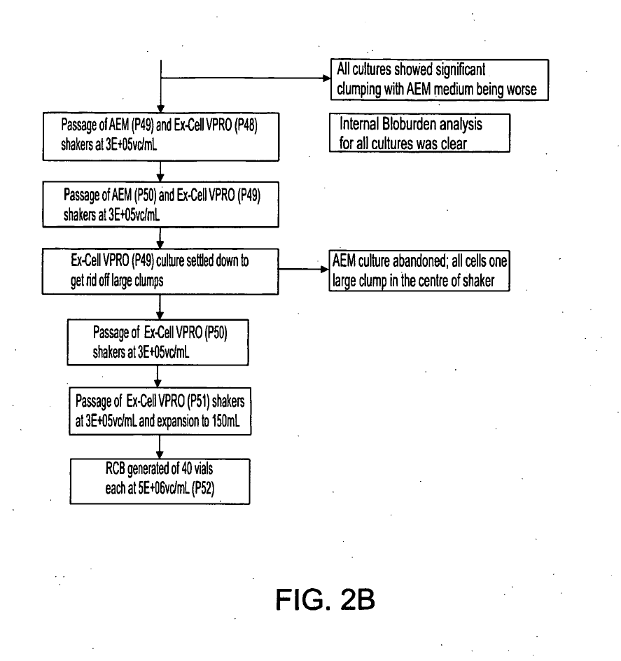

FIGS. 2A-B are flow charts that summarize the adaptation steps of adherent WCB to the RCB in suspension. Suspended MCB is generated from the RCB, as outlined in Table 7, below.

TABLE 7
Preparation of Master Cell Bank (Procedure)
Cell Thawing
Thaw 1 ampoule of 1 ml RCB PER.C6 cells at 37° C. in a water bath.
Slowly add 9 ml of pre-warmed growth medium to the thawed cells. Centrifuge at 210 g at 22° C. for 5
minutes. Discard the supernatant; add 10 ml of growth medium
Seed cells in a T75 cm² flask. Incubate at 37 ± 2° C. for 3 days.
↓
Cell Expansion

| | |
|---|---|
| First Passage: | Incubate 4-T75 cm² flasks at 37 ± 2° C. for 3 days at a density of 3.0 × 10⁵ viable |
| | cells/ml. |
| Second passage: | Pool the cells, perform one passage to 3 × 250 ml and 1 × 500 ml Erlenmeyer |
| | flasks. Incubate by shaking at 90 rpm, at 37 ± 2° C. for 3 days. |
| Third Passage: | Pool the cells, passage to 4 × 1 L Erlenmeyer flasks. Incubate by |
| | shaking at 90 rpm, at 37 ± 2° C. for 4 days. |
| Fourth Passage: | Pool the cells, passage to 6 × 2 L Erlenmeyer flasks. Incubate by |
| | shaking at 90 rpm, at 37 ± 2° C. for 3 days. |

↓
Preparation of MCB
Pool the cells and centrifuge for 5 min at 210 g at 4° C.
Total Cell bank concentration is 1.4 × 10⁹ cells.
An equal volume (140 ml) of cell bank ( 1.0 × 10⁷ viable cells/ml)
is mixed with 140 ml of freezing mix ×2 medium.
Aliquot the cell bank to 1.1 ml aliquots in a final concentration of 5.0 × 10⁶ viable cells/ml,
Keep frozen at liquid N₂.

TABLE 8
specification of the non-adherent MCB.

| Test | Result |
|---|---|
| Growth testing and viability of PER.C6 cell banks | First vial—8.01 × 10⁵ cells/mL, viability 89.7%. |
| | Last vial 8.08 × 10⁵ cells/mL, % viability 90.7%. |
| Cell growth and sample preparation | Satisfactory growth |
| Sterility testing by direct inclusion method (EP, | Ne bacteria or fungi detected |
| JP and USP) harmonized version | |
| Qualification of test article material for sterility | Test article successfully qualified |
| by direct inoculation method (EP, JP, USP) | |
| Mycoplasma detection, EP (Vero, broth and agar | No mycoplasma not micoplasma activity detected |
| with inhibition assay) | |
| In vitro detection of viral contaminants (3 detector | No evidence of presence of viral contaminants |
| cell lines—MRC-5, Vero and HeLa) | |
| Identification and Characterization of cultured | Isoenzyme migration distances observed were |
| cells by analysis of 6 isoenzymes | consistent with those expected |
| In vitro assay using suckling mice, adult mice, | No evidence of presence of viral contaminants |
| guinea pigs and embryonated eggs | |

### Example 3

### Construction of the PPE1-3X-Fas-c Chimera

pWE.Ad.AfAfIII-rITRsp Backbone Cosmid, is a 40.5 kb cosmid, purchased from Crucell. This backbone contains most of the genome of adenovirus type 5, as well as partial homology to the pAdAdpt5 adaptor plasmid, which enables recombination.

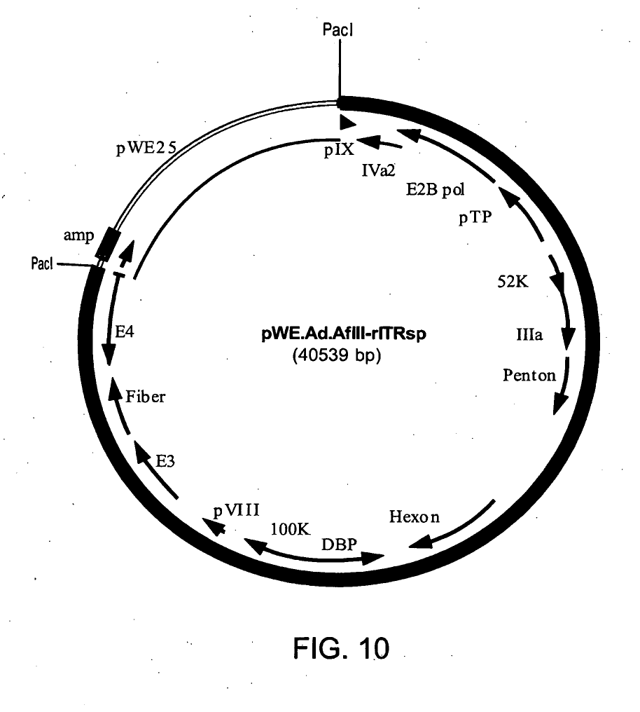

The E1 early transcriptional unit was deleted from the backbone plasmid (pWE.Ad.Afiii-rlTRsp). The cosmid was digested with Pad restriction enzyme deleting the pWE25 and the Amp resistance selection marker site (see FIG. 10).

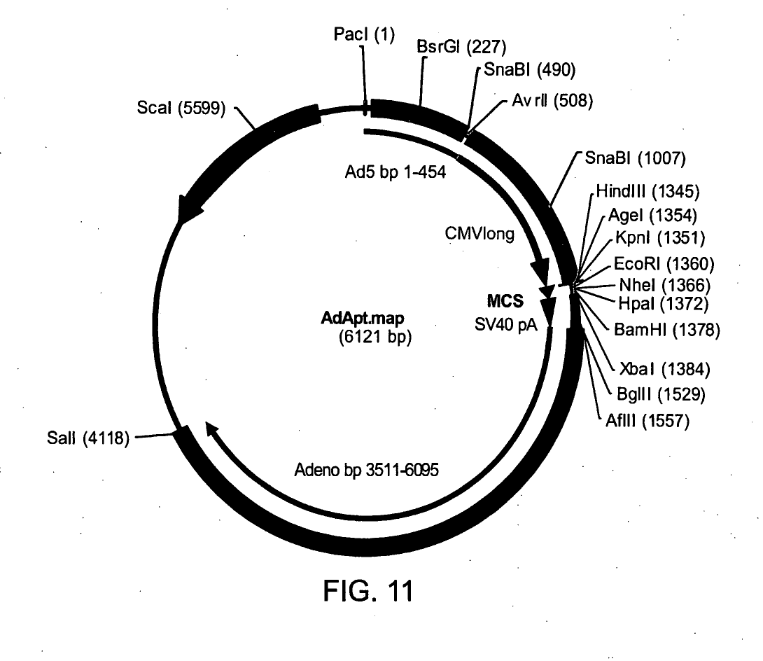

The Adaptor Plasmid—The pAdApt plasmid, 6121 bp, contains sequences of the Ad5, CMV promoter, MCS, and SV40 polyA (see FIG. 11).

The plasmid was digested at the SnaB1 and EcoR1 sites deleting the CMV promoter. These sites were used to insert the PPE and Fas-c fragment.

Gene Insert

Restricted expression of the transgene to those tissues that endogenously recognize the promoter PPE-1—the angiogenic endothelial cells is based in the PPE-1-3x, a modified version of the PPE-1 promoter. PPE-1-3x, further induces specificity to angiogenic vessels. The modified promoter contains three copies of the 43 bp regulatory region. Two copies were added in the same direction as in the wild-type promoter and the third was split in two and the order of the two fragments was inversed. The modified promoter was utilized for construction of the adenoviral vector. (See SEQ ID NO: 7)

The transgene of the invention contains a unique human Fas-chimera (Fas-c) pro-apoptotic transgene, under the control of the PPE-1 promoter. This chimera is composed of the extra cellular and intra membranal domains of the human TNF-R1 (Tumor Necrosis Factor Receptor 1, SEQ ID NO: 2) and of the Fas (p55) intracellular domain (SEQ ID NO: 3, Boldin et al, JBC, 1995). Fas gene has been shown to effectively induce cell death both in endothelial and in non-endothelial cells.

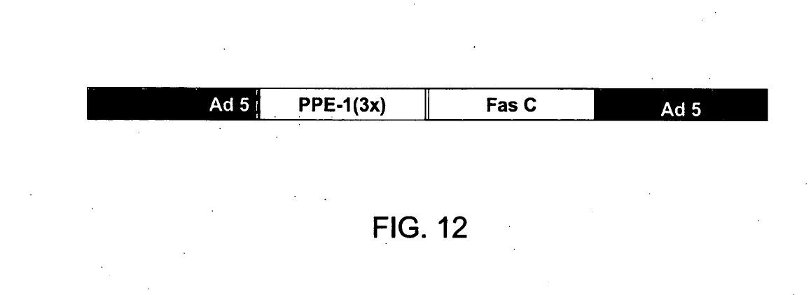

The PPE-1-(3X)-Fas-c element (2115 bp) was constructed from the PPE-1-(3X)-luc element. This element contains the 1.4 kb of the murine preproendothelin PPE-1-(3X) promoter, the Luciferase gene, the SV40 polyA site and the first intron of the murine ET-1 gene, originated from the pEL8 plasmid (8848 bp) used by Harats et al (Harats D. et al., JCI, 1995). The PPE-3-Luc cassette was extracted from the pEL8 plasmid using the BamHI restriction enzyme. The Luciferase gene was substituted by the Fas-c gene to obtain the PPE-1-3x-Fas-c cassette as shown in FIG. 12.

pACPPE-1(3x)-Fas-c Plasmid—The cassette was further introduced into the backbone plasmid pACCMV.pLpA using the BamH1 sites, resulting with the pACPPE-1(3 x)-Fas-c plasmid.

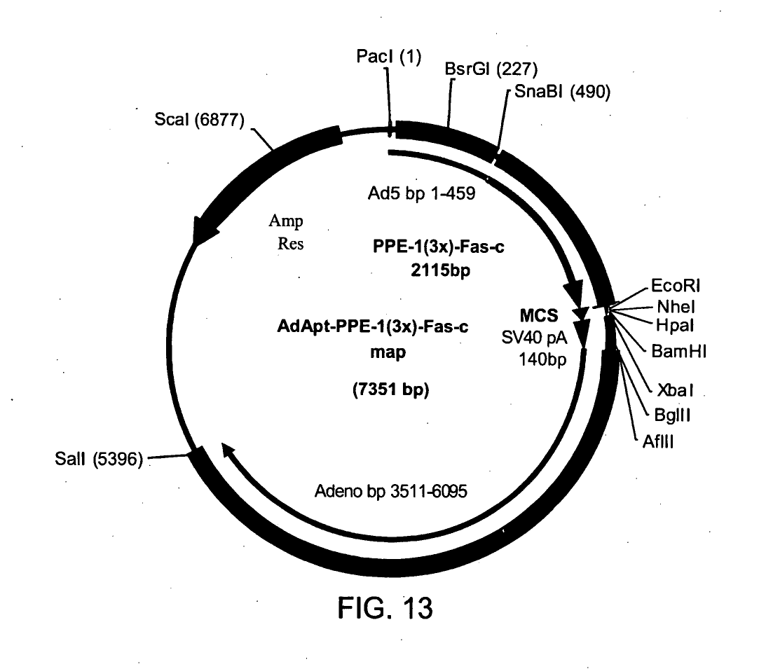

AdApt-PPE-1(3x)-Fas-c Plasmid—The PPE-1-3x-Fas-c element was extracted from the first generation construct, pACPPE-1-3x-Fas-c, and was amplified with the SnaB1 and EcoR1 PCR primers introducing SnaB1 and EcoR1 sites at the 5′-and-3′-end respectively. These sites were used to clone the PPE-Fas-c fragment into pAdApt digested with SnaB1 and EcoR1 resulting with the AdApt-PPE-1-3x-Fas-c used for transfection of the PER.C6 cells (see FIG. 13).

### Example 4

### Seed Stock

PER.C6 cells were co-transfected to generate Ad5.PPE.Fas-c virus vector using lipofectamine mediated transfection with the linearized (Pac 1/Sal 1 digested) pAdApt.PPE.Fas-c plasmid and the linearized (Pac 1 digested) backbone cosmid pWE.Ad.AfAfIII-rITRsp to produce second generation final product. Generation of the virus was established by full cytopathic effect (CPE) shown by the virus vector.

For each of the product generations viral seed stocks were prepared. At the end of the plaque purification process the plaques were identified as the final product by PCR and found sterile and Mycoplasma free. The chosen plaque was shown to be RCA free, as well.

### Example 5

### Production

For the production process employing adherent cells grown with serum: a WCB vial is thawed, seeded in growth media, and expanded. Cells are infected with the VB-111 MVB. The virus is harvested after 72 hr of incubation, with 90% CPE. The media is centrifuged and the pellet collected. Freezing and thawing releases the virus particles from the cells, followed by an additional centrifugation to remove cell debris.

TABLE 9
Cell Thawing and Expansion
In a class 100 biosafety cabinet (BSC) with a class 10,000 room background (Cell Culture Room)
Thawing of PER.C6 cells from Working Cell Bank
Seeding in Growth Medium
Incubation
↓
Repeated expansions through incubation to 70% confluence
Cells are transferred to Virus Culture Room

TABLE 10
Viral Infection of Cells and Propagation of Virus
In a class 100 BSC with a class 10,000 room background
(Virus Culture Room)
Thawing of virus from Master Viral Bank
Viral infection in 55-70% confluent cells
Viral propagation through incubation
↓
90%-100% CPE
Centrifugation and collection of pellet (Initially Clarified Harvest)
Freeze and thaw
Centrifugation and collection of supernatant (Clarified Harvest)
Harvest frozen at ≦−65° C.

For the production process employing non-adherent cells in serum free medium: the production process includes suspending the expanded PER.C6 cells in Erlenmeyer flasks followed by an expansion in a 10 L Cultibag (wave bag) and an expansion in the final 50 L wave bag (total 25 L).

Recovery of virus from cells is achieved by lysis using detergents such as Triton X-100. For a process based on disposable systems the detergent lysis is the preferred option as it does not require capital investment, cleaning validation. Addition of 10% Triton 3-4 days post infection for 1 hr incubation at 37° C. in 17 rpm (into the cultibag wave)

Removal of host DNA is achieved by the addition of Benzonase (15 IU/ml) and 1 mM of MgCl₂ for 1 hr incubation at 37° C. in 17 rpm (into the cultibag wave)

TABLE 11
Cell Thawing and Expansion non adherent process

| | |
|---|---|
| Thawing of PER.C6 Cells from Master Cell Bank | UPSTREAM |
| Thaw two vials from the MCB at a 37° C. water bath. Transfer each vial to a 50 ml tube and add | |
| 9 ml of warmed growth medium to each tube. Centrifuge at 850 rpm for 5 minutes at RT. | |
| Seed into 2 × T75 cm² flasks. | |
| Incubate in a static position at 37 ± 2° C. for 3-4 days. | |
| ↓ | |
| Static Culture in T-75 cm² flask | |
| Pool the cells and seed into 5 × T-75 cm² flasks at a concentration of 3 × 10⁵ viable cells/ml. | |
| Incubate in a static position at 37 ± 2° C. for 3 days. | |
| ↓ | |
| Cell Culture in 500 ml Erlenmeyer shaking flasks | |
| Pool the cells, seed into 2 × 500 m1 Erlenmeyer shaking flasks at a concentration of | |
| 3 × 10⁵ viable cells/ml. | |
| Incubate at 37 ± 2° C. by shaking rotation of 90 rpm for 3-4 days. | |
| ↓ | |
| Cell Culture in 2 L Erlenmeyer shaking flasks | |
| Pool the cells, seed into 2 × 2 L Erlenmeyer flasks at a concentration of 3 × 10⁵ viable cells/ml | |
| Incubate at 37 ± 2° C., by rocking at 90 rpm for 3-4 days. | |
| Repeat this step only by seeding into at least 5 × 2 L Erlenmeyer flasks. | |
| ↓ | |
| Cell Culture in 10 L or 50 L Cultibag | |
| Setup of the BIOSTAT 10 L/50 L Cultibag and BIOSTAT control tower. | |
| Transfer the filtered complete growth medium into the wave bag (3 L for 10 L wave | |
| and 20 L for the 50 L wave). | |
| Set the rocker to 17 rpm, temperature to 37° C., gas flow to 300 cc and CO₂ to 10%. | |
| Pool the cells and perform a cell count. | |
| Seed at a concentration of 4 × 10⁵ viable cells/ml for a total of 2 × 10⁹ cells in a 10 L | |
| Cultibag and 1 × 10¹⁰ cells in a 50 L Cultibag. | |
| Incubate at 37° C., 17 rpm, gas flow 300 cc and 10% CO₂. | |
| ↓ | |

TABLE 12

| Viral Infection of Cells and Propagation of Virus non adherent process | |
|---|---|
| Virus Infection of PER.C6 Cells | UPSTREAM |
| Infect 25 L of 1-1.5 × 10⁶/ml viable PER.C6 cells with WVB or MVB at MOI 5.0 and incubate | |
| for 72-96 hours at 37 ± 2° C., by rocking at 17 rpm, pH 7.0, | |
| DO₂ 30% | |
| ↓ | |
| Harvest and Cell Lysis | |
| Stop the DO₂ and the pH control, add 2.8 L of 10% Triton X-100. Incubate for 1 hour at 37 ± 2° C., | |
| by rocking at 17 rpm. | |
| Add 15 IU/ml of Benzonase and 1 mM of MgCl₂ to the complete growth media (100 ml total), | |
| incubate for 1 hour at 37 ± 2° C., by rocking at 17 rpm | |

Comparison between adherent and suspension PER.C6 cells (Crucell) process with respect to multiple parameters is provided in Table 13, below.

TABLE 13

| | Adherent Process | | Suspension Process | |
|---|---|---|---|---|
| | Cell | | Cell | |
| Stage | viability | Seeding concentration/Vessel | viability | Seeding concentration/Vessel |
| Thawing | 95-99% | Starting with 1 ampoule (1 CC) | 92% | Starting with 2 (2 CC) ampoules |
| | | from CB | | from CB |
| | | (WCBP6001 P-39, | | (CTL 2008#015ON, |
| | | 5 × 10⁶ cells/1 ml) | | 5 × 10⁶ cells/ml) |
| | | Seeding T-25 cm² flask with 5 ml | | Seeding 2 × T-75 cm² flasks with |
| | | of 6 × 10⁵-1 × 10⁶ cells/ml | | 10 m1 of 3 × 10⁵ cells/ml per flask |
| | | 2 harvests (5 ml total per | | 4 days in culture (20 ml total) |
| | | harvest) 4 days in culture (10 ml, | | |
| | | total) | | |
| Expansion | 95-99% | Seeding 1 × T-75 cm² flask with | 89% | Seeding 5 × T-75 cm² flasks with |
| 1 | | 15 ml of 2.7-4.7 × 10⁵ cells/ml per | | 10 ml of 3 × 10⁵ cells/ml per flask |
| | | flask | | 3-4 days in culture (50 ml, |
| | | 4-6 days in culture (30 ml, | | 1.5 × 10⁷ cells total) |
| | | 10⁷ cells total of 2 harvests) | | |
| Expansion | 95-99% | Seeding 5 × T-75 cm2 flasks with | 87% | Seeding 2 × 500 ml Erlenmeyer |
| 2 | | 15 ml of 2.7-4.7 × 10⁵ cells/ml per | | with 75 ml of 3 × 10⁵ cells/ml per |
| | | flask | | Erlenmeyer |
| | | 4-6 days in culture (150 ml, | | 3-4 days in culture (150 ml |
| | | 5.5 × 10⁷ cells total of 2 harvests) | | 4.5 × 10⁷ cells total) |
| Expansion | 95-99% | Seeding 12 × T-150 cm² flasks | 98% | Seeding 2 × 2 L Erlenmeyer with |
| 3 | | with 30 ml of 2.7- | | 400 ml of 3 × 10⁵ cells/ml per |
| | | 4.7 × 10⁵ cells/ml per flask 4-6 | | Erlenmeyer |
| | | days in culture | | 3-4 days in culture (800 ml, |
| | | (720 ml, 2.6 × 10⁸ cells total of 2 | | 2.4 × 10⁸ cells total) |
| | | harvests) | | |
| Expansion | 95-99% | Seeding 30 × T-300 cm² flasks | 99% | Seeding 5 × 2 L Erlenmeyer with |
| 4 | | with 120 ml of 2.3- | | 400 ml of 3 × 10⁵ cells/ml per |
| | | 6.6 × 10⁵ cells/ml per flask | | Erlenmeyer |
| | | 4-6 days in culture | | 3-4 days in culture (2 L, |
| | | (3.6 L, 1.5 × 10⁹ cells total of 2 | | 0.6 × 10⁹ cells total |
| | | harvests) | | |
| Expansion | 95-99% | Seeding 150 × T-300 flasks with | 99% | Seeding 10 L Cultibag wave 5 L |
| 5 | | 60 ml of 2.3-6.6 × 10⁵ cells/ml per | | of 4 × 10⁵/ml |
| | | flask | | 4 days in culture (5 L, |
| | | 4-6 days in culture (18 L, 8 × 10⁹ | | tota1, 2 × 10⁹ cells total) |
| | | cells total of 2 harvests) | | |
| Expansion | NA | NA | 96.9% | Seeding 50 L Cultibag wave with |
| 6 | | Final volume 18L, ~3.5- | | 25 L of 4 × 10⁵/ml |
| | | 5 × 10¹⁰ cells | | 3 days in culture (25 L, 10¹⁰ cells |
| | | | | total,) |
| | | | | Final volume of 25 L, - |
| | | | | ~3.9 × 10¹⁰ cells |

### Example 6

### Optimization of Viral Titre (for VB-III Produced in Non-Adherent Serum Free Cells)

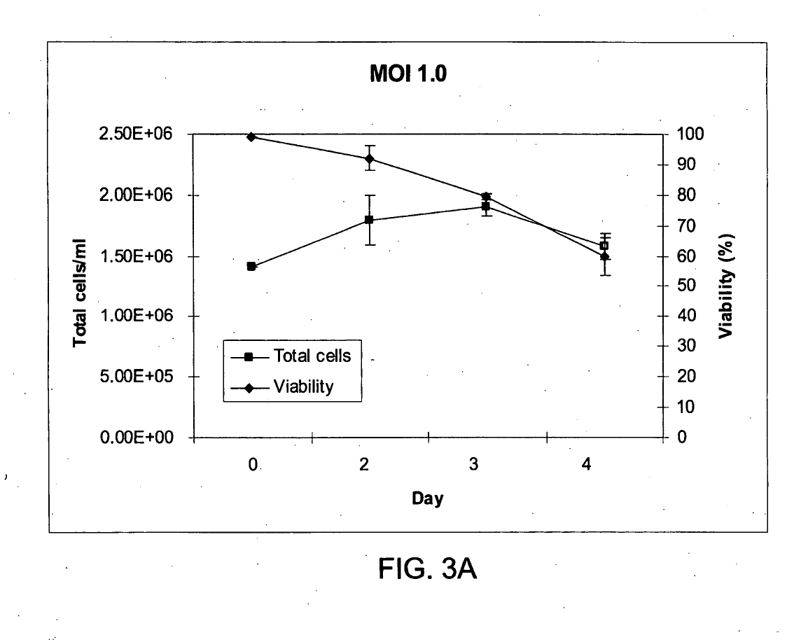

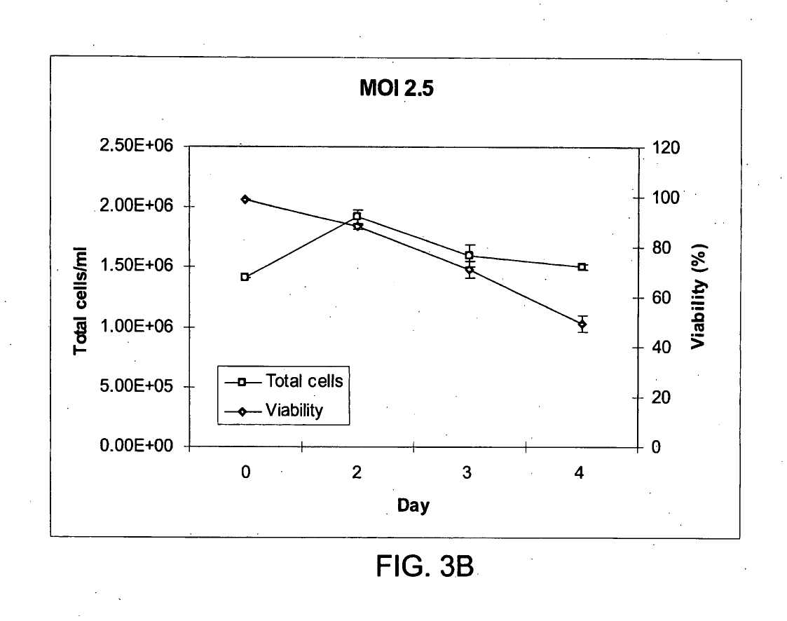

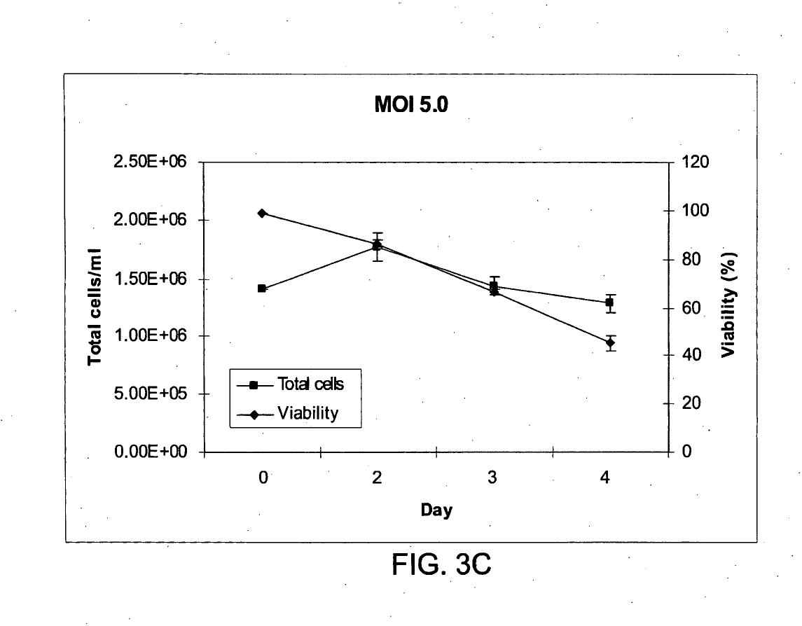

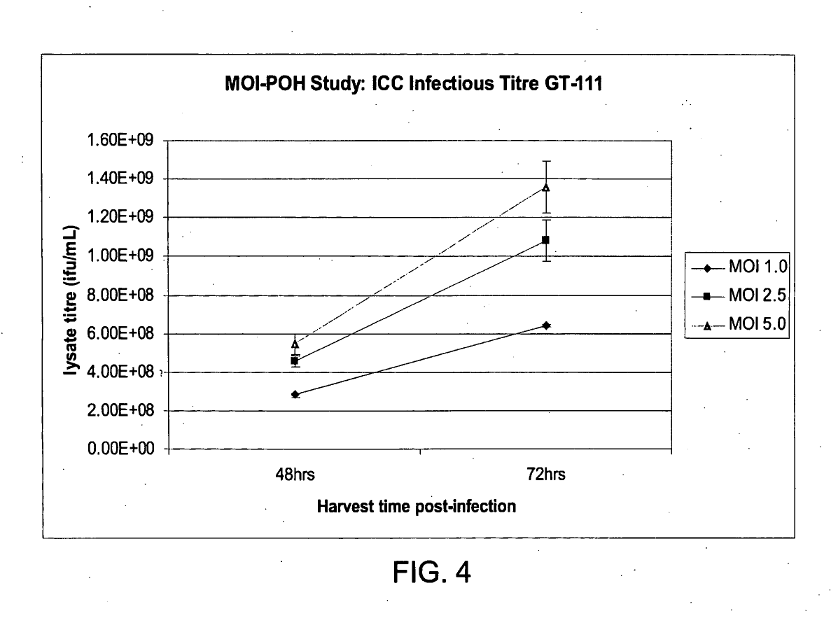

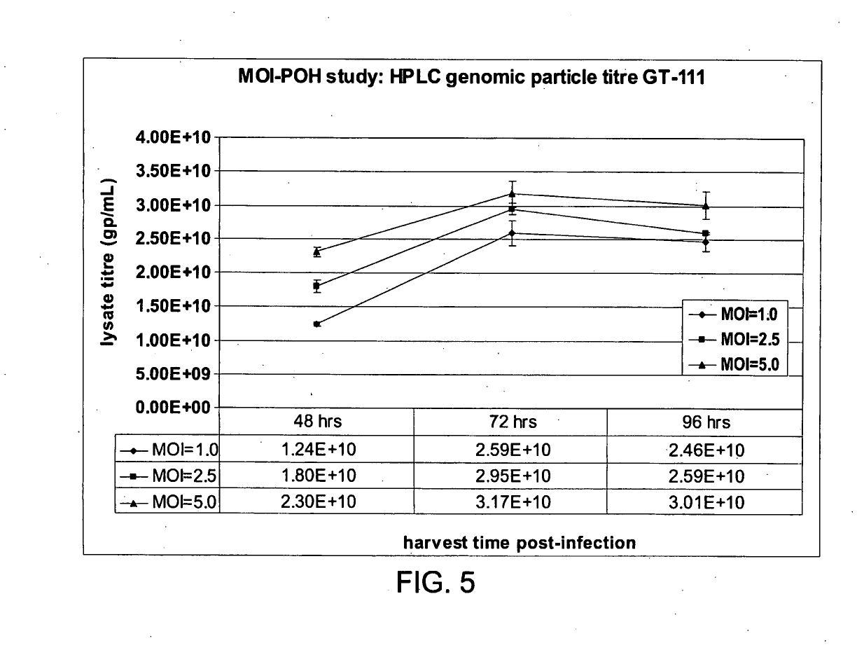

It is usually necessary to optimize conditions around the quantity of virus used in the infection of the producer cells and in the determination of the optimal point of harvest for the cell culture. Therefore, it is normal to optimize the MOI (multiplicity of infection) and point of harvest (POH) values within the development programs so as to optimize the viral productivity. The optimisation of MOI for the virus production process using the generated RCB PER.C6 and VBL's MVBP611 as infection material. MOIs 1, 2.5 and 5 and harvest points of 48 and 72 hrs were evaluated (FIGS. 3-4). Samples at 96 hrs harvest point were also generated were assayed only for genomic titre by HPLC (FIG. 5). The recommended MOI is 5 and the POH is 3 days post infection.

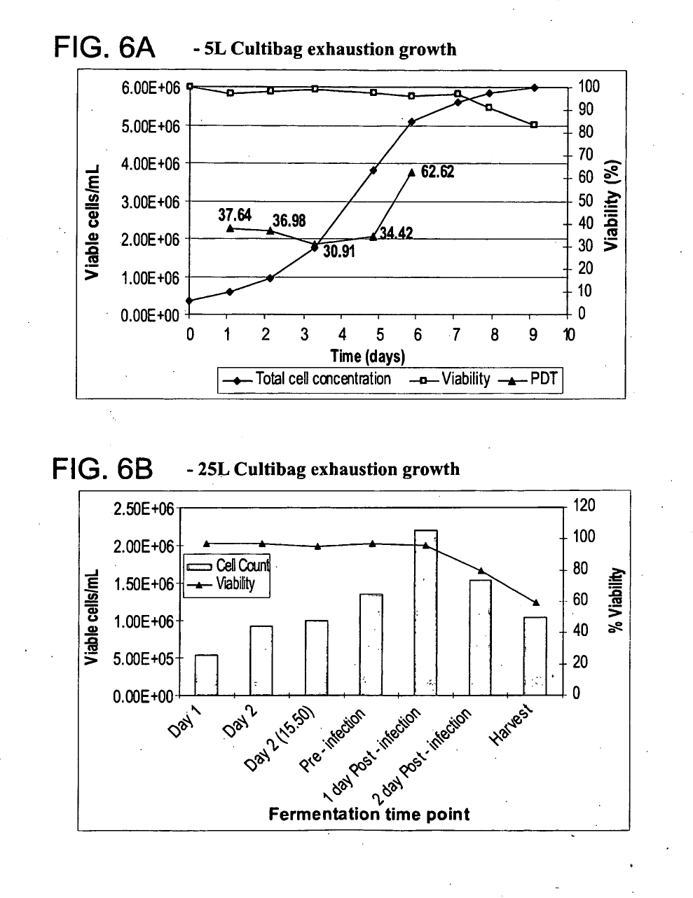

Growing PER.C6 in Sartorius CultiBag™ wave technology, comparable growth curves and virus productivity were seen at the 5 L and 25 L scale. FIGS. 6A-B show a typical PER.C6 exhaustion cell growth study at the 5 L scale and growth combined with to VB111 infection/production at the 25 L scale. PER.C6 cells grow to about 6×10⁶ viable cells/mL with consistent high viability. When infected at reasonable multiplicity of infection (MOI), cell growth is inhibited soon after infection.

### Example 7

### Downstream—for Production in Adherent Cells Grown with Serum

The downstream process includes centrifugation on a discontinuous CsCl gradient followed by centrifugation on a continuous CsCl gradient. This stage is essential in order to remove defective particles and proteins present in the cell lysate, as well as media, serum and cellular debris and to concentrate the virus to a level suitable for injection. The residual Cs is removed by two rounds of Sephadex desalting columns (elution of the virus is done with PBS).

TABLE 14
Virus Purification
In a class 100 BSC with a class 10,000 room background (Virus Culture Room)
Clarified Harvest thawed*
Virus loaded on discontinuous gradient (CsCl d1.2 & d1.4)
Centrifugation
↓
Virus collection
Virus loaded on continuous gradient (CsCl d1.2 to d1.4)
Centrifugation
↓
Virus collection
Removal of CsCl residues with 2 rounds on a Sephadex column
*Two Clarified Harvests may be combined at this point, to produce a larger batch, following appropriate testing

TABLE 15
Sterile Filtration and Filling
In a class 100 BSC with a class 10,000 room background (Filling Room)
Dilution to required concentration (vp/ml) with a solution of
PBS with 10% glycerol
↓
Sterile filtration and filling 0.5-0.6 ml in cryovials
Batch stored at ≦−65° C.

### Example 8

### Downstream for Production in Non-Adherent Serum Free Cells

### Recovery and Purification by Ion Exchange and Size Exclusion Chromatography

After infection and harvesting, clarification is done and purification of the virus is done using Gel Permeation Chromatography (GPC) and ion exchange (IEX) columns (500 ml 10¹² VP/ml purified material).

The next process step is removal of cell debris, which at small scale <500 L, can normally be achieved using depth filtration. The scale of filters required for development scale processes means that disposable units can be used through out and once established it is possible to apply the same filter train for a range of products.

Having obtained a clarified feedstock, the next step applied is an ultrafiltration step. This has three functions: firstly, it allows the process volumes to be significantly reduced; secondly, the process media can be exchanged for an optimal buffer system for the initial capture chromatography step and thirdly, due to the very large size of the viral vectors, it is possible to use high cut-off molecular weight membranes ≦300 Kd that not only allow for the removal of the lysis detergent from the product stream, but also a significant portion of the low molecular weight contaminants, including the digested nucleic acid, and a significant amount of the host protein. This step can therefore also be regarded as a key purification operation.

TABLE 16
Virus Clarification

| | |
|---|---|
| Harvest Filtration | DOWNSTREAM |
| Harvest the material through clarification filters | |
| and adjust the salt concentration to 500 mM by adding | |
| 3M NaCl solution | |
| ↓ | |
| Diafiltrate the material on a Hollow fiber TFF (TFF1) using UFP-300-E-55 HF | |
| cartridge | |
| (2.1 m², 60 cm, 1 mm lumen) | |
| Load on an IEX chromatography column containing | |
| Q Sepharose XI virus licensed | |
| ↓ | |
| Load on a GPC chromatography column containing Sepahrose 4FF | |
| ↓ | |
| Diafiltrate the material on Hollow fiber TFF (TFF2) using UFP-300-C-4A HF | |
| cartridge | |
| (0.065 m², 30 cm, 0.5 mm lumen) | |
| ↓ | |
| Filtrate the material in PBS + 10% Glycerol using a 0.45 μm filter, aliquot into final | |
| containers and remove QC and retain samples from the last container filled. | |
| ↓ | |
| Store the Bulk Drug Substance aliquots, QC samples and retains at ≦−65° C. | |

TABLE 17
Sterile Filtration and Filling
Sterile filtration is done using a 0.2 μm PES sterile mini Capsule filter,
and filling of 1.1 ml aliquots into 1.8 ml cryovials.
The Final Product is stored at ≦−65° C.

This operation can be performed with hollow fiber tangential flow system. With regards to development operations it is critical that optimal concentration factors are determined for specific viral constructs as over-concentration can lead to product precipitation.

Having performed the initial recovery operations, the next process stages are chromatographic purification. The aims of these purification steps are predominantly to remove host and product related contaminants from the product, rather than achieve separation of infective and non-infective viral particles.

The capture step is performed with a packed bed anion exchange chromatographic step. Here, the resin choice is critical to obtain high purities and product recoveries. In addition, it is necessary to optimize product elution conditions for different viral constructs to ensure high recoveries and purities. The process currently uses Q-Sepharose-XL from GE Healthcare. As the loading of the virus onto the chromatography resin is known to be a critical parameter with regards to process recoveries and purities, the dynamic resin capacity should be confirmed/determined for each new virus product as should potential wash steps to enhance the clearance of impurities. With binding chromatographic operations, it is also necessary to ensure that appropriate steps are taken to stabilize the virus during this process step. For example, product concentrations may be very high during the elution from binding chromatographic steps and the virus may also be exposed to high salt concentrations.

These types of events may result in aggregation of the virus and significant product loss during later stages of processing.

The second chromatographic step applied is a size exclusion step run as a group separation where up to 30% of the column volume is loaded and the virus is collected at the excluded fraction. Due to the large size of the virus it is possible to use very large pore size resins, which allows for the complete removal of the “low molecular weight” (e.g., <1,000 Kd) particles, and also exchange of the viral product into the required formulation buffer. A typical OD₂₆₀/OD₂₈₀ trace is shown in FIGS. 7A-B.

### Example 9

### Comparisons of 5 L and 25 L Process Scale Runs

### Production in Non-Adherent Serum Free Cells

With regards to overall process performance both yield and product quality have been retained (Table 17, below), including the clearance of critical impurities such as residual Triton X100, Benzonase and host DNA. From data such as this it is possible to conclude that the outlined VB 111 process is robust and suitable for the production of clinical grade material.

TABLE 18
QC testing results summary for 5 L development, 25 L toxicity and 25 L
cGMP batch materials.

| | 5 L verification run | 25 L technical run | 25 L cGMP batch |
|---|---|---|---|
| Harvest genomic titre | 3.93 × 10¹⁰ gp/mL | 7.78 × 10¹⁰ gp/mL | not available |
| (HPLC) | | | |
| Harvest infectious titre | not available | 2.2 × 10⁹ ifu/mL | not available |
| (ICC) | | | |
| Drug substance/product | 1.52 × 10¹² gp/mL | 2.02 × 10¹² gp/mL | 1.7 × 10¹² gp/mL |
| titre (HPLC) | | | |
| Drug substance/product | 2.45 × 10¹⁰ ifu/mL | 6.4 × 10¹⁰ ifu/mL | 1.2 × 10¹⁰ ifu/mL |
| (ICC) | | | |
| SDS-PAGE | conforms to reference | conforms to reference | conforms to reference |
| (identity/purity) | | | |
| Endotoxin | not available | 31.1 EU/10¹³ vp | 1.63 EU/10¹³ vp |
| Benzonase ELISA | not available | not available | <0.1 ng/mL |
| Residual Triton X-100 | not available | none detected | none detected |
| Bioburden | not available | 0 cfu/mL | 0 cfu/mL |
| PH | not available | 7.2 | 7.2 |
| residual host DNA by | 72 pg/1.0 × 10¹¹ gp | 16.8 pg/1.0 × 10¹¹ gp | <45.9 pg/1.0 × 10¹¹ gp |
| QPCR | | | |
| Overall process yield | ~40% | ~52% | not available |

### Example 10

### Construction and Characterization of the AdPPE-1(3x)-TK Vector

The HSV-TK/GCV is the most widely studied and implemented cytoreductive gene-drug combination. Cells transfected with an HSV-TK-containing plasmid or transduced with an HSV-TK containing vector, are made sensitive to the drug super-family including aciclovir, ganciclovir (GCV), valciclovir and famciclovir. The guanosine analog GCV is the most active drug in combination with TK. HSV-TK positive cells produce a viral TK, which is three orders of magnitude more efficient in phosphorylating GCV into GCV monophosphate (GCV-MP) than the human TK. GCV-MP is subsequently phosphorylated by the native thymidine kinase into GCV diphosphate and finally to GCV triphosphate (GCV-TP).

Constructing an Adenovirus-5 Vector Armed with the HSV-TK Gene Controlled by the Modified Murine Pre-Proendothelin-1 Promoter.

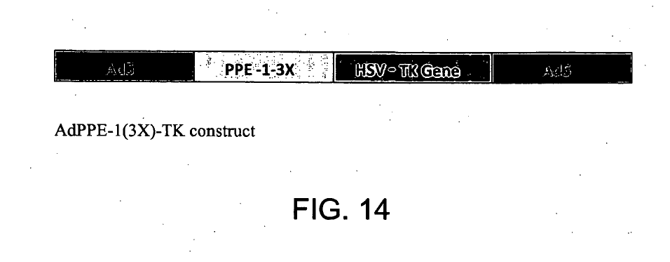

The replication-deficient vector, designated AdPPE-1(3x)-TK, was constructed on the basis of a first generation (E1 gene deleted, E3 incomplete) adenovirus-5 vector. The recombinant vector was prepared by co-transfection of the plasmids pACPPE-1(3x)-TK (described in details in WO2008/132729) and pJM-17 (40.3 kb, WO2008/132729) in human embryonal kidney-293 (HEK-293) using well-known conventional cloning techniques. The pJM-17 plasmid contains the entire adenovirus-5 genome except for the E1 gene. The HEK-293 cell line substitutes the E1 deletions, since they contain an E1 gene in trans. One out of 40 homologous recombinations induced the vector AdPPE-1(3x)-TK. FIG. 14 shows a schematic map of the vector AdPPE-1(3x)-TK. The specific sequence of the PPE-1(3x) is as described in Example 3 of the Fas-c chimera vector. Clinical samples of the vector (AdPPE-1(3x)-TK) are generated using PER.C6 cells as described above.

### Example 11

### Conditionally Replicating Adenovirus Vectors

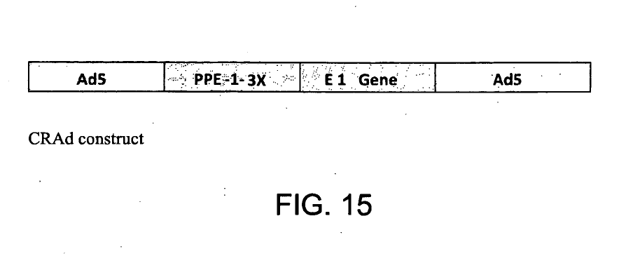

The CRADs were constructed as described in WO2008/132729, which is hereby incorporated by reference in its entirety. Briefly, the plasmids were constructed using the AdEasy method (Stratagene, LaJolla Calif.). PShuttle-MK, a plasmid containing parts of the adenovirus-5 DNA sequence, has been modified as follows: the multiple cloning site and right arm in pShuttle (Stratagene, La Jolla, Calif.) were replaced by Midkine (Ink) promoter and the consecutive adenoviral E1 region. Later, the MK promoter was replaced by PPE1-3x without intron. A second plasmid was constructed by subcloning IRES sequence (from p IRES-EYFP plasmid, BD Biosciences) and FAS-chimera cDNA between the promoter and E1. IRES permits translation of two proteins from the same transcript. The resultant two shuttles were linearized with PmeI digestion and subsequently transformed into Escherichia coli BJ5183ADEASY-1 (Stratagem). This type of bacteria has already been transformed with pADEASY-1 plasmid, which contains most of the adenovirus-5 sequence, except E1 and E3 gene regions. The plasmids undergo homologous recombination within the bacteria (between pShuttle and pADEASY-1), thus creating the complete vector genome (see exemplary schematic FIG. 15). The recombinants were later Pad digested and transfected with calcium phosphate method into 293 human embryonic kidney cell-line (ATCC). Clinical samples are generated using the PER.C6 cells as described for the Fas-c above.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for producing an adenovirus, the method comprising culturing, in a serum-free suspension culture or in an adherent culture, PER.C6 cells infected with an adenovirus comprising a murine pre-proendothelin promoter, whereby said adenovirus is produced.

2. The method of claim 1, wherein said adenovirus is selected from the group consisting of a non-replicating adenovirus and a conditionally replicating adenovirus.

3. The method of claim 2, wherein said non-replicating adenovirus comprises a polynucleotide which comprises a fas-chimera transgene transcriptionally linked to said murine pre-proendothelin promoter.

4. The method of claim 2, wherein said conditionally replicating adenovirus is transcriptionally linked to said murine pre-proendothelin promoter.

5. The method of claim 2, wherein said non-replicating adenovirus comprises a polynucleotide which comprises an anti-angiogenic transgene transcriptionally linked to said murine pre-proendothelin promoter.

6. The method of claim 2, wherein said non-replicating adenovirus comprises a polynucleotide which comprises a pro-angiogenic transgene transcriptionally linked to said murine pre-proendothelin promoter.

7. The method of claim 2, wherein said non-replicating adenovirus comprises a polynucleotide which comprises a suicide transgene transcriptionally linked to said murine pre-proendothelin promoter.

8. The method of claim 2, wherein said adenovirus is a conditionally replicating adenovirus that is transcriptionally linked to said murine pre-proendothelin promoter, and wherein said adenovirus is devoid of non-viral heterologous sequences encoding pro- or anti-angiogenic agents.

9. The method of claim 7, wherein said suicide transgene comprises a thymidine kinase.

10. The method of claim 1, wherein said adenovirus further comprises a heterologous nucleic acid sequence encoding a therapeutic agent operably linked to said murine pre-proendothelin promoter.

11. The method of claim 10, wherein said heterologous nucleic acid sequence comprises an apoptotic gene.

12. The method of claim 1, further comprising recovering virus from said cells following said culturing.

13. The method of claim 12, wherein said recovering is effected at a point of harvest (POH) of 3-4 days post infection and an MOI of 5.

14. The method of claim 1, wherein said culturing is effected at a 5-200 L volume.

15-18. (canceled)

19. The method of claim 12, wherein said recovering is effected by subjecting said cells to a detergent lysis.

20. The method of claim 19, wherein said detergent comprises Triton X-100.

21. The method of claim 19, further comprising removing cellular DNA and cell debris so as to obtain a clear feedstock.

22. The method of claim 21, wherein said feedstock is subjected to Tangential Flow Filtration (TFF).

23. The method of claim 22, further comprising obtaining a viral pellet and subjecting the viral pellet to anion exchange chromatography and size exclusion chromatography.

24. The method of claim 3, wherein said fas-chimera transgene comprises a polynucleotide having a nucleotide sequence selected from the group consisting of SEQ ID NO 2, SEQ ID NO 3 and SEQ ID NO: 4.

25-26. (canceled)

27. The method of claim 1, wherein said murine pre-pro endothelin promoter further comprises a polynucleotide having a nucleotide sequence selected from the group consisting of SEQ ID NO: 5, SEQ ID NO: 6 or the antisense sequence thereof, SEQ ID NO: 7 or the antisense sequence thereof, SEQ ID NO: 8 or the antisense sequence thereof, and an combinations thereof.

28.-32. (canceled)

33. The method of claim 27, wherein said murine pre-pro endothelin promoter comprises a polynucleotide having a nucleotide sequence as set forth in SEQ ID NO: 12.

34. The method of claim 3, wherein said non-replicating adenovirus vector is an adenovirus 5 vector.

35. The method of claim 34, wherein said adenovirus 5 vector comprises a nucleic acid sequence as set forth in SEQ ID NO: 9 or 10.

36.-50. (canceled)

51. The method of claim 12, wherein said recovering is effected by freeze-thaw releasing of the virus.

52. The method of claim 21, wherein said removing cellular DNA and cell debris is effected by ultracentrifugation.

53.-67. (canceled)

68. A viral preparation generated according to the method of claim 1, wherein said viral preparation exhibits (a) ion exchange and size exclusion chromatography traces of FIGS. 7A-B and a product profile of Table 6, or (b) the product profile of Table 3.

69. (canceled)

70. A pharmaceutical composition comprising as an active ingredient the viral preparation of claim 68.

71. A method of reducing angiogenesis in a subject in need thereof, the method comprising administering to the subject a therapeutically effective amount of the viral preparation of claim 68, thereby reducing angiogenesis in the subject.

72. The method of claim 71, wherein the subject has a solid tumor.

73. The method of claim 71, wherein said administering intravenous administration.

74. The method of claim 1, wherein said method of producing is a large scale method.

75. The method of claim 74, wherein the method starts with a culture volume of 5-100 L.


SEQUENCE LISTING (646 sequences)

SEQ ID NO: 1 - DNA, 34350 bp, Artificial sequence
  Empty Ad5 vector sequence without repeats
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtacgtctcgagcatgcatctaggcggccgcatggcagaaattcgcgaattcgctagcgttaacggatcctctagacgagatccgaacttgtttattgcagcttataatggttacaaataaagcaatagcatcacaaatttcacaaataaagcatttttttcactgcattctagttgtggtttgtccaaactcatcaatgtatcttatcatgtctagatctgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgctttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 2 - DNA, 590 bp, Artificial sequence
  TNFR portion of the TNFR-Fas chimera (Fas-c)
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctacc

SEQ ID NO: 3 - DNA, 511 bp, Artificial sequence
  Fas portion of the TNFR-Fas chimera (Fas-c)
aagcttaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 4 - DNA, 1101 bp, Artificial sequence
  TNFR1-Fas chimera (Fas-c) coding sequence
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccaagcttaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 5 - DNA, 6 bp, Artificial sequence
  Hypoxia responsive element - E-box
gcacgt

SEQ ID NO: 6 - DNA, 44 bp, Artificial sequence
  Murine endothelial specific enhancer elemet
gtacttcatacttttcattccaatggggtgactttgcttctgga

SEQ ID NO: 7 - DNA, 143 bp, Artificial sequence
  A triplicate copy of a murine enhancer sequence originated from the PPE-1 promoter
gtacttcatacttttcattccaatggggtgactttgcttctggagggtgactttgcttctggagccagtacttcatacttttcattgtacttcatacttttcattccaatggggtgactttgcttctggaggctagctgccag

SEQ ID NO: 8 - DNA, 47 bp, Artificial sequence
  EDC fragment
ctggagggtgactttgcttctggagccagtacttcatacttttcatt

SEQ ID NO: 9 - DNA, 36460 bp, Artificial sequence
  PPE-1-3X-FasC virl construct including repeat sequences
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtacgtacgtgtacttctgatcggcgatactagggagataaggatgtacctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcctccagaagcaaagtcaccccattggaatgaaaagtatgaagtacaatgaaaagtatgaagtactggctccagaagcaaagtcaccctccagaagcaaagtcaccccattggaatgaaaagtatgaagtacgctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaataaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgctaattctgcagatcgggatccggcatgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccaagcttaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagctcgagcatgcatctaggcggccgcatggcagaaattcgcgaattcgctagcgttaacggatcctctagacgagatccgaacttgtttattgcagcttataatggttacaaataaagcaatagcatcacaaatttcacaaataaagcatttttttcactgcattctagttgtggtttgtccaaactcatcaatgtatcttatcatgtctagatctgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgctttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 10 - DNA, 35203 bp, Artificial sequence
  PPE-1-3X-FasC virl construct (lacking repeats)
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtacgtacgtgtacttctgatcggcgatactagggagataaggatgtacctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcctccagaagcaaagtcaccccattggaatgaaaagtatgaagtacaatgaaaagtatgaagtactggctccagaagcaaagtcaccctccagaagcaaagtcaccccattggaatgaaaagtatgaagtacgctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaataaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgctaattctgcagatcgggatccggcatgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccaagcttaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagctcgagcatgcatctaggcggccgcatggcagaaattcgcgaattcgctagcgttaacggatcctctagacgagatccgaacttgtttattgcagcttataatggttacaaataaagcaatagcatcacaaatttcacaaataaagcatttttttcactgcattctagttgtggtttgtccaaactcatcaatgtatcttatcatgtctagatctgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgctttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgat

SEQ ID NO: 11 - DNA, 33093 bp, Artificial sequence
  Empty Ad5 vector sequence  (repeats included)
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtacgtctcgagcatgcatctaggcggccgcatggcagaaattcgcgaattcgctagcgttaacggatcctctagacgagatccgaacttgtttattgcagcttataatggttacaaataaagcaatagcatcacaaatttcacaaataaagcatttttttcactgcattctagttgtggtttgtccaaactcatcaatgtatcttatcatgtctagatctgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgctttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgat

SEQ ID NO: 12 - DNA, 978 bp, Artificial sequence
  PPE-1-3X promoter
acgtgtacttctgatcggcgatactagggagataaggatgtacctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcctccagaagcaaagtcaccccattggaatgaaaagtatgaagtacaatgaaaagtatgaagtactggctccagaagcaaagtcaccctccagaagcaaagtcaccccattggaatgaaaagtatgaagtacgctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaataaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctg

SEQ ID NO: 13 - DNA, 1334 bp, Mus musculus
gtgtgtgtgtgtgtgtgtgtgtgtgtgtgtgtgtgtgtgtgtgtagtgtacttctgatcggcgatactagggagataaggatgtacctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaataaagtcagagctgtttacccccactctataggggttcaatataaaaaggcggcggagaactgtccgagtcagacgcgttcctgcaccggcgctgagagcctgacccggtctgctccgctgtccttgcgcgctgcctcccggctgcccgcgacgctttcgccccagtggaagggccacttgctgaggaccgcgctgagatctaaaaaaaaaacaaaaaacaaaaaacaaaaaaacccagaggcgatcagagcgaccagacaccgtcctcttcgttttgcattgagttccatttgcaaccgagttttctttttttcctttttccccactcttctgacccctttgcagaatggattattttcccgtgatcttctctctgctgttcgtgactttccaaggagctccagaaacaggtaggcgccacttgcgaatctttctacttcagcgcagcagttatcgcttctgttttccacttttctttctttcttttctttcattctttcctttttatttatttttttaattactgaagctccagcagcaagtgccttacaattaattaacttctgtgtgaagcgaaagaaataaaacccctgtttgaatacagctgactacaaccgagtatcgcatagcttc

SEQ ID NO: 14 - DNA, 5 bp, Artificial sequence
  wild type element M9
ctgga

SEQ ID NO: 15 - DNA, 5 bp, Artificial sequence
  wild type element M4
cattc

SEQ ID NO: 16 - DNA, 5 bp, Artificial sequence
  wild type element M5
caatg

SEQ ID NO: 17 - DNA, 5 bp, Artificial sequence
  wild type element M6
gggtg

SEQ ID NO: 18 - DNA, 5 bp, Artificial sequence
  wild type element M7
acttt

SEQ ID NO: 19 - DNA, 5 bp, Artificial sequence
  wild type element M8
gcttc

SEQ ID NO: 20 - DNA, 5 bp, Artificial sequence
  wild type element M1
gtact

SEQ ID NO: 21 - DNA, 5 bp, Artificial sequence
  wild type element M3
ctttt

SEQ ID NO: 22 - DNA, 19 bp, Artificial sequence
  Exemplary element X variant
gggtgactttgcttctgga

SEQ ID NO: 23 - DNA, 19 bp, Artificial sequence
  Exemplary element X variant
gggtgactttgcttctgga

SEQ ID NO: 24 - DNA, 19 bp, Artificial sequence
  Exemplary element X variant
ggggtgactttgcttctgg

SEQ ID NO: 25 - DNA, 19 bp, Artificial sequence
  Exemplary element X variant
caatggggtggcttctgga

SEQ ID NO: 26 - DNA, 19 bp, Artificial sequence
  Exemplary element X variant
ccaatggggtggcttctgg

SEQ ID NO: 27 - DNA, 19 bp, Artificial sequence
  Exemplary element X variant
gggtgactttgcttctgga

SEQ ID NO: 28 - DNA, 19 bp, Artificial sequence
  Exemplary element X variant
ggggtgactttgcttctgg

SEQ ID NO: 29 - DNA, 44 bp, Artificial sequence; identical to SEQ ID NO: 6
  Exemplary element X variant

SEQ ID NO: 30 - DNA, 39 bp, Artificial sequence
  Exemplary element X variant
gtactcttttcattccaatggggtgactttgcttctgga

SEQ ID NO: 31 - DNA, 39 bp, Artificial sequence
  Exemplary element X variant
gtacttcatacattccaatggggtgactttgcttctgga

SEQ ID NO: 32 - DNA, 39 bp, Artificial sequence
  Exemplary element X variant
gtacttcatacttttcaatggggtgactttgcttctgga

SEQ ID NO: 33 - DNA, 39 bp, Artificial sequence
  Exemplary element X variant
gtacttcatacttttcattcgggtgactttgcttctgga

SEQ ID NO: 34 - DNA, 34 bp, Artificial sequence
  Exemplary element X variant
gtactcattccaatggggtgactttgcttctgga

SEQ ID NO: 35 - DNA, 29 bp, Artificial sequence
  Exemplary element X variant
gtactcaatggggtgactttgcttctgga

SEQ ID NO: 36 - DNA, 24 bp, Artificial sequence
  Exemplary element X variant
gtactgggtgactttgcttctgga

SEQ ID NO: 37 - DNA, 34 bp, Artificial sequence
  Exemplary element X variant
gtacttcatacaatggggtgactttgcttctgga

SEQ ID NO: 38 - DNA, 29 bp, Artificial sequence
  Exemplary element X variant
gtacttcatagggtgactttgcttctgga

SEQ ID NO: 39 - DNA, 34 bp, Artificial sequence
  Exemplary element X variant
gtacttcatacttttgggtgactttgcttctgga

SEQ ID NO: 40 - DNA, 34 bp, Artificial sequence
  Exemplary element X variant
gtactcttttcaatggggtgactttgcttctgga

SEQ ID NO: 41 - DNA, 34 bp, Artificial sequence
  Exemplary element X variant
gtacttcatacattcgggtgactttgcttctgga

SEQ ID NO: 42 - DNA, 34 bp, Artificial sequence
  Exemplary element X variant
gtactcttttcattcgggtgactttgcttctgga

SEQ ID NO: 43 - DNA, 44 bp, Artificial sequence; identical to SEQ ID NO: 6
  Exemplary element X variant

SEQ ID NO: 44 - DNA, 39 bp, Artificial sequence; identical to SEQ ID NO: 30
  Exemplary element X variant

SEQ ID NO: 45 - DNA, 39 bp, Artificial sequence; identical to SEQ ID NO: 31
  Exemplary element X variant

SEQ ID NO: 46 - DNA, 39 bp, Artificial sequence; identical to SEQ ID NO: 32
  Exemplary element X variant

SEQ ID NO: 47 - DNA, 39 bp, Artificial sequence; identical to SEQ ID NO: 33
  Exemplary element X variant

SEQ ID NO: 48 - DNA, 39 bp, Artificial sequence
  Exemplary element X variant
gtacttcatacttttcattccaatgactttgcttctgga

SEQ ID NO: 49 - DNA, 39 bp, Artificial sequence
  Exemplary element X variant
gtacttcatacttttcattccaatggggtggcttctgga

SEQ ID NO: 50 - DNA, 34 bp, Artificial sequence; identical to SEQ ID NO: 34
  Exemplary element X variant

SEQ ID NO: 51 - DNA, 29 bp, Artificial sequence
  Exemplary element X variant
gtactcaatggggtgactttgcttctgga

SEQ ID NO: 52 - DNA, 24 bp, Artificial sequence
  Exemplary element X variant
gtactgggtgactttgcttctgga

SEQ ID NO: 53 - DNA, 19 bp, Artificial sequence
  Exemplary element X variant
gtactactttgcttctgga

SEQ ID NO: 54 - DNA, 34 bp, Artificial sequence; identical to SEQ ID NO: 37
  Exemplary element X variant

SEQ ID NO: 55 - DNA, 28 bp, Artificial sequence
  Exemplary element X variant
cattccaatggggtgactttgcttctgg

SEQ ID NO: 56 - DNA, 23 bp, Artificial sequence
  Exemplary element X variant
cattcgggtgactttgcttctgg

SEQ ID NO: 57 - DNA, 19 bp, Artificial sequence
  Exemplary element X variant
cattcactttgcttctgga

SEQ ID NO: 58 - DNA, 19 bp, Artificial sequence
  Exemplary element X variant
cttttcattcgcttctgga

SEQ ID NO: 59 - DNA, 23 bp, Artificial sequence
  Exemplary element X variant
cattccaatgactttgcttctgg

SEQ ID NO: 60 - DNA, 19 bp, Artificial sequence
  Exemplary element X variant
cattccaatggcttctgga

SEQ ID NO: 61 - DNA, 23 bp, Artificial sequence
  Exemplary element X variant
cattccaatggggtggcttctgg

SEQ ID NO: 62 - DNA, 19 bp, Artificial sequence
  Exemplary element X variant
cattcgggtggcttctgga

SEQ ID NO: 63 - DNA, 23 bp, Artificial sequence
  Exemplary element X variant
caatggggtgactttgcttctgg

SEQ ID NO: 64 - DNA, 19 bp, Artificial sequence
  Exemplary element X variant
caatggggtggcttctgga

SEQ ID NO: 65 - DNA, 19 bp, Artificial sequence
  Exemplary element X variant
caatgactttgcttctgga

SEQ ID NO: 66 - DNA, 19 bp, Artificial sequence
  Exemplary element X variant
cattccaatggcttctgga

SEQ ID NO: 67 - DNA, 28 bp, Artificial sequence
  Exemplary element X variant
cattccaatggggtgactttgcttctgg

SEQ ID NO: 68 - DNA, 23 bp, Artificial sequence
  Exemplary element X variant
cattccaatgactttgcttctgg

SEQ ID NO: 69 - DNA, 19 bp, Artificial sequence
  Exemplary element X variant
cattccaatggcttctgga

SEQ ID NO: 70 - DNA, 23 bp, Artificial sequence
  Exemplary element X variant
cattccaatggggtggcttctgg

SEQ ID NO: 71 - DNA, 43 bp, Artificial sequence
  Exemplary element X variant
gtacttcatacttttcattccaatggggtgactttgcttctgg

SEQ ID NO: 72 - DNA, 43 bp, Artificial sequence; identical to SEQ ID NO: 71
  Exemplary element X variant

SEQ ID NO: 73 - DNA, 38 bp, Artificial sequence
  Exemplary element X variant
gtactcttttcattccaatggggtgactttgcttctgg

SEQ ID NO: 74 - DNA, 38 bp, Artificial sequence
  Exemplary element X variant
gtacttcatacattccaatggggtgactttgcttctgg

SEQ ID NO: 75 - DNA, 33 bp, Artificial sequence
  Exemplary element X variant
gtactcattccaatggggtgactttgcttctgg

SEQ ID NO: 76 - DNA, 43 bp, Artificial sequence; identical to SEQ ID NO: 71
  Exemplary element X variant

SEQ ID NO: 77 - DNA, 38 bp, Artificial sequence
  Exemplary element X variant
gtacttcatacttttcattcgggtgactttgcttctgg

SEQ ID NO: 78 - DNA, 38 bp, Artificial sequence
  Exemplary element X variant
gtacttcatacttttcattccaatgactttgcttctgg

SEQ ID NO: 79 - DNA, 38 bp, Artificial sequence
  Exemplary element X variant
gtacttcatacttttcattccaatggggtggcttctgg

SEQ ID NO: 80 - DNA, 33 bp, Artificial sequence
  Exemplary element X variant
gtacttcatacttttcattcactttgcttctgg

SEQ ID NO: 81 - DNA, 33 bp, Artificial sequence
  Exemplary element X variant
gtacttcatacttttcattcgggtggcttctgg

SEQ ID NO: 82 - DNA, 28 bp, Artificial sequence
  Exemplary element X variant
gtacttcatacttttcattcgcttctgg

SEQ ID NO: 83 - DNA, 33 bp, Artificial sequence
  Exemplary element X variant
gtacttcatacttttcattccaatggcttctgg

SEQ ID NO: 84 - DNA, 33 bp, Artificial sequence
  Exemplary element X variant
gtacttcatacattcgggtgactttgcttctgg

SEQ ID NO: 85 - DNA, 33 bp, Artificial sequence
  Exemplary element X variant
gtacttcatacattccaatgactttgcttctgg

SEQ ID NO: 86 - DNA, 33 bp, Artificial sequence
  Exemplary element X variant
gtacttcatacattccaatggggtggcttctgg

SEQ ID NO: 87 - DNA, 28 bp, Artificial sequence
  Exemplary element X variant
gtacttcatacattcactttgcttctgg

SEQ ID NO: 88 - DNA, 28 bp, Artificial sequence
  Exemplary element X variant
gtacttcatacattcgggtggcttctgg

SEQ ID NO: 89 - DNA, 23 bp, Artificial sequence
  Exemplary element X variant
gtacttcatacattcgcttctgg

SEQ ID NO: 90 - DNA, 28 bp, Artificial sequence
  Exemplary element X variant
gtacttcatacattccaatggcttctgg

SEQ ID NO: 91 - DNA, 38 bp, Artificial sequence; identical to SEQ ID NO: 73
  Exemplary element X variant

SEQ ID NO: 92 - DNA, 33 bp, Artificial sequence
  Exemplary element X variant
gtactcttttcattcgggtgactttgcttctgg

SEQ ID NO: 93 - DNA, 33 bp, Artificial sequence
  Exemplary element X variant
gtactcttttcattccaatgactttgcttctgg

SEQ ID NO: 94 - DNA, 33 bp, Artificial sequence
  Exemplary element X variant
gtactcttttcattccaatggggtggcttctgg

SEQ ID NO: 95 - DNA, 28 bp, Artificial sequence
  Exemplary element X variant
gtactcttttcattcactttgcttctgg

SEQ ID NO: 96 - DNA, 28 bp, Artificial sequence
  Exemplary element X variant
gtactcttttcattcgggtggcttctgg

SEQ ID NO: 97 - DNA, 23 bp, Artificial sequence
  Exemplary element X variant
gtactcttttcattcgcttctgg

SEQ ID NO: 98 - DNA, 28 bp, Artificial sequence
  Exemplary element X variant
gtactcttttcattccaatggcttctgg

SEQ ID NO: 99 - DNA, 33 bp, Artificial sequence; identical to SEQ ID NO: 75
  Exemplary element X variant

SEQ ID NO: 100 - DNA, 28 bp, Artificial sequence
  Exemplary element X variant
gtactcattcgggtgactttgcttctgg

SEQ ID NO: 101 - DNA, 28 bp, Artificial sequence
  Exemplary element X variant
gtactcattccaatgactttgcttctgg

SEQ ID NO: 102 - DNA, 28 bp, Artificial sequence
  Exemplary element X variant
gtactcattccaatggggtggcttctgg

SEQ ID NO: 103 - DNA, 23 bp, Artificial sequence
  Exemplary element X variant
gtactcattcactttgcttctgg

SEQ ID NO: 104 - DNA, 23 bp, Artificial sequence
  Exemplary element X variant
gtactcattcgggtggcttctgg

SEQ ID NO: 105 - DNA, 23 bp, Artificial sequence
  Exemplary element X variant
gtactcattccaatggcttctgg

SEQ ID NO: 106 - DNA, 43 bp, Artificial sequence; identical to SEQ ID NO: 71
  Exemplary element X variant

SEQ ID NO: 107 - DNA, 38 bp, Artificial sequence; identical to SEQ ID NO: 73
  Exemplary element X variant

SEQ ID NO: 108 - DNA, 38 bp, Artificial sequence; identical to SEQ ID NO: 74
  Exemplary element X variant

SEQ ID NO: 109 - DNA, 38 bp, Artificial sequence
  Exemplary element X variant
gtacttcatacttttcaatggggtgactttgcttctgg

SEQ ID NO: 110 - DNA, 33 bp, Artificial sequence; identical to SEQ ID NO: 75
  Exemplary element X variant

SEQ ID NO: 111 - DNA, 33 bp, Artificial sequence
  Exemplary element X variant
gtacttcatacaatggggtgactttgcttctgg

SEQ ID NO: 112 - DNA, 33 bp, Artificial sequence
  Exemplary element X variant
gtactcttttcaatggggtgactttgcttctgg

SEQ ID NO: 113 - DNA, 28 bp, Artificial sequence
  Exemplary element X variant
gtactcaatggggtgactttgcttctgg

SEQ ID NO: 114 - DNA, 38 bp, Artificial sequence; identical to SEQ ID NO: 78
  Exemplary element X variant

SEQ ID NO: 115 - DNA, 38 bp, Artificial sequence; identical to SEQ ID NO: 79
  Exemplary element X variant

SEQ ID NO: 116 - DNA, 33 bp, Artificial sequence; identical to SEQ ID NO: 83
  Exemplary element X variant

SEQ ID NO: 117 - DNA, 33 bp, Artificial sequence; identical to SEQ ID NO: 93
  Exemplary element X variant

SEQ ID NO: 118 - DNA, 33 bp, Artificial sequence; identical to SEQ ID NO: 85
  Exemplary element X variant

SEQ ID NO: 119 - DNA, 33 bp, Artificial sequence
  Exemplary element X variant
gtacttcatacttttcaatgactttgcttctgg

SEQ ID NO: 120 - DNA, 28 bp, Artificial sequence
  Exemplary element X variant
gtactcattccaatgactttgcttctgg

SEQ ID NO: 121 - DNA, 28 bp, Artificial sequence
  Exemplary element X variant
gtacttcatacaatgactttgcttctgg

SEQ ID NO: 122 - DNA, 28 bp, Artificial sequence
  Exemplary element X variant
gtactcttttcaatgactttgcttctgg

SEQ ID NO: 123 - DNA, 23 bp, Artificial sequence
  Exemplary element X variant
gtactcaatgactttgcttctgg

SEQ ID NO: 124 - DNA, 33 bp, Artificial sequence; identical to SEQ ID NO: 94
  Exemplary element X variant

SEQ ID NO: 125 - DNA, 33 bp, Artificial sequence; identical to SEQ ID NO: 86
  Exemplary element X variant

SEQ ID NO: 126 - DNA, 33 bp, Artificial sequence
  Exemplary element X variant
gtacttcatacttttcaatggggtggcttctgg

SEQ ID NO: 127 - DNA, 28 bp, Artificial sequence
  Exemplary element X variant
gtactcattccaatggggtggcttctgg

SEQ ID NO: 128 - DNA, 28 bp, Artificial sequence
  Exemplary element X variant
gtacttcatacaatggggtggcttctgg

SEQ ID NO: 129 - DNA, 28 bp, Artificial sequence
  Exemplary element X variant
gtactcttttcaatggggtggcttctgg

SEQ ID NO: 130 - DNA, 23 bp, Artificial sequence
  Exemplary element X variant
gtactcaatggggtggcttctgg

SEQ ID NO: 131 - DNA, 28 bp, Artificial sequence
  Exemplary element X variant
gtactcttttcattccaatggcttctgg

SEQ ID NO: 132 - DNA, 28 bp, Artificial sequence
  Exemplary element X variant
gtacttcatacattccaatggcttctgg

SEQ ID NO: 133 - DNA, 28 bp, Artificial sequence
  Exemplary element X variant
gtacttcatacttttcaatggcttctgg

SEQ ID NO: 134 - DNA, 23 bp, Artificial sequence
  Exemplary element X variant
gtactcattccaatggcttctgg

SEQ ID NO: 135 - DNA, 23 bp, Artificial sequence
  Exemplary element X variant
gtacttcatacaatggcttctgg

SEQ ID NO: 136 - DNA, 23 bp, Artificial sequence
  Exemplary element X variant
gtactcttttcaatggcttctgg

SEQ ID NO: 137 - DNA, 43 bp, Artificial sequence; identical to SEQ ID NO: 71
  Exemplary element X variant

SEQ ID NO: 138 - DNA, 38 bp, Artificial sequence; identical to SEQ ID NO: 73
  Exemplary element X variant

SEQ ID NO: 139 - DNA, 38 bp, Artificial sequence; identical to SEQ ID NO: 74
  Exemplary element X variant

SEQ ID NO: 140 - DNA, 33 bp, Artificial sequence; identical to SEQ ID NO: 75
  Exemplary element X variant

SEQ ID NO: 141 - DNA, 38 bp, Artificial sequence; identical to SEQ ID NO: 78
  Exemplary element X variant

SEQ ID NO: 142 - DNA, 38 bp, Artificial sequence; identical to SEQ ID NO: 79
  Exemplary element X variant

SEQ ID NO: 143 - DNA, 33 bp, Artificial sequence; identical to SEQ ID NO: 83
  Exemplary element X variant

SEQ ID NO: 144 - DNA, 33 bp, Artificial sequence; identical to SEQ ID NO: 93
  Exemplary element X variant

SEQ ID NO: 145 - DNA, 33 bp, Artificial sequence; identical to SEQ ID NO: 85
  Exemplary element X variant

SEQ ID NO: 146 - DNA, 28 bp, Artificial sequence
  Exemplary element X variant
gtactcattccaatgactttgcttctgg

SEQ ID NO: 147 - DNA, 33 bp, Artificial sequence; identical to SEQ ID NO: 94
  Exemplary element X variant

SEQ ID NO: 148 - DNA, 33 bp, Artificial sequence; identical to SEQ ID NO: 86
  Exemplary element X variant

SEQ ID NO: 149 - DNA, 28 bp, Artificial sequence
  Exemplary element X variant
gtactcattccaatggggtggcttctgg

SEQ ID NO: 150 - DNA, 28 bp, Artificial sequence
  Exemplary element X variant
gtactcttttcattccaatggcttctgg

SEQ ID NO: 151 - DNA, 28 bp, Artificial sequence
  Exemplary element X variant
gtacttcatacattccaatggcttctgg

SEQ ID NO: 152 - DNA, 23 bp, Artificial sequence
  Exemplary element X variant
gtactcattccaatggcttctgg

SEQ ID NO: 153 - DNA, 43 bp, Artificial sequence; identical to SEQ ID NO: 71
  Exemplary element X variant

SEQ ID NO: 154 - DNA, 19 bp, Artificial sequence
  Exemplary element X variant
acttttcattccaatgggg

SEQ ID NO: 155 - DNA, 19 bp, Artificial sequence
  Exemplary element X variant
cttttcattccaatggggt

SEQ ID NO: 156 - DNA, 19 bp, Artificial sequence
  Exemplary element X variant
ttttcattccaatggggtg

SEQ ID NO: 157 - DNA, 19 bp, Artificial sequence
  Exemplary element X variant
tttcattccaatggggtga

SEQ ID NO: 158 - DNA, 19 bp, Artificial sequence
  Exemplary element X variant
tacttttcattccaatggg

SEQ ID NO: 159 - DNA, 19 bp, Artificial sequence
  Exemplary element X variant
atacttttcattccaatgg

SEQ ID NO: 160 - DNA, 19 bp, Artificial sequence
  Exemplary element X variant
catacttttcattccaatg

SEQ ID NO: 161 - DNA, 19 bp, Artificial sequence
  Exemplary element X variant
tcatacttttcattccaat

SEQ ID NO: 162 - DNA, 19 bp, Artificial sequence
  Exemplary element X variant
ttcatacttttcattccaa

SEQ ID NO: 163 - DNA, 19 bp, Artificial sequence
  Exemplary element X variant
ttccaatggggtgactttg

SEQ ID NO: 164 - DNA, 19 bp, Artificial sequence
  Exemplary element X variant
attccaatggggtgacttt

SEQ ID NO: 165 - DNA, 19 bp, Artificial sequence
  Exemplary element X variant
cattccaatggggtgactt

SEQ ID NO: 166 - DNA, 19 bp, Artificial sequence
  Exemplary element X variant
tcattccaatggggtgact

SEQ ID NO: 167 - DNA, 19 bp, Artificial sequence
  Exemplary element X variant
ttcattccaatggggtgac

SEQ ID NO: 168 - DNA, 19 bp, Artificial sequence
  Exemplary element X variant
tttcattccaatggggtga

SEQ ID NO: 169 - DNA, 19 bp, Artificial sequence
  Exemplary element X variant
ttttcattccaatggggtg

SEQ ID NO: 170 - DNA, 19 bp, Artificial sequence
  Exemplary element X variant
cttttcattccaatggggt

SEQ ID NO: 171 - DNA, 19 bp, Artificial sequence
  Exemplary element X variant
acttttcattccaatgggg

SEQ ID NO: 172 - DNA, 24 bp, Artificial sequence
  Exemplary element X variant
tttcattccaatggggtgactttg

SEQ ID NO: 173 - DNA, 24 bp, Artificial sequence
  Exemplary element X variant
ttttcattccaatggggtgacttt

SEQ ID NO: 174 - DNA, 24 bp, Artificial sequence
  Exemplary element X variant
cttttcattccaatggggtgactt

SEQ ID NO: 175 - DNA, 24 bp, Artificial sequence
  Exemplary element X variant
acttttcattccaatggggtgact

SEQ ID NO: 176 - DNA, 24 bp, Artificial sequence
  Exemplary element X variant
tacttttcattccaatggggtgac

SEQ ID NO: 177 - DNA, 24 bp, Artificial sequence
  Exemplary element X variant
atacttttcattccaatggggtga

SEQ ID NO: 178 - DNA, 24 bp, Artificial sequence
  Exemplary element X variant
catacttttcattccaatggggtg

SEQ ID NO: 179 - DNA, 24 bp, Artificial sequence
  Exemplary element X variant
tcatacttttcattccaatggggt

SEQ ID NO: 180 - DNA, 24 bp, Artificial sequence
  Exemplary element X variant
ttcatacttttcattccaatgggg

SEQ ID NO: 181 - DNA, 19 bp, Artificial sequence
  Exemplary element X variant
ttttcattccaatggggtg

SEQ ID NO: 182 - DNA, 19 bp, Artificial sequence
  Exemplary element X variant
tttcattccaatggggtga

SEQ ID NO: 183 - DNA, 19 bp, Artificial sequence
  Exemplary element X variant
attccaatggggtgacttt

SEQ ID NO: 184 - DNA, 19 bp, Artificial sequence
  Exemplary element X variant
ttccaatggggtgactttg

SEQ ID NO: 185 - DNA, 19 bp, Artificial sequence
  Exemplary element X variant
cattccaatggggtgactt

SEQ ID NO: 186 - DNA, 19 bp, Artificial sequence
  Exemplary element X variant
tcattccaatggggtgact

SEQ ID NO: 187 - DNA, 19 bp, Artificial sequence
  Exemplary element X variant
ttcattccaatggggtgac

SEQ ID NO: 188 - DNA, 19 bp, Artificial sequence
  Exemplary element X variant
tttcattccaatggggtga

SEQ ID NO: 189 - DNA, 19 bp, Artificial sequence
  Exemplary element X variant
ttttcattccaatggggtg

SEQ ID NO: 190 - DNA, 24 bp, Artificial sequence
  Exemplary element X variant
ttttcattccaatggggtgacttt

SEQ ID NO: 191 - DNA, 25 bp, Artificial sequence
  Exemplary element X variant
tcatacttttcattccaatggggtg

SEQ ID NO: 192 - DNA, 23 bp, Artificial sequence
  Exemplary element X variant
tttcattccaatggggtgacttt

SEQ ID NO: 193 - DNA, 19 bp, Artificial sequence
  Exemplary element X variant
ttttcattcgggtgacttt

SEQ ID NO: 194 - DNA, 24 bp, Artificial sequence
  Exemplary element X variant
ttttcattccaatgactttgcttc

SEQ ID NO: 195 - DNA, 19 bp, Artificial sequence
  Exemplary element X variant
ttttcattcactttgcttc

SEQ ID NO: 196 - DNA, 19 bp, Artificial sequence
  Exemplary element X variant
attccaatggggtgacttt

SEQ ID NO: 197 - DNA, 19 bp, Artificial sequence
  Exemplary element X variant
attccaatgactttgcttc

SEQ ID NO: 198 - DNA, 19 bp, Artificial sequence
  Exemplary element X variant
ttccaatgactttgcttct

SEQ ID NO: 199 - DNA, 24 bp, Artificial sequence
  Exemplary element X variant
tttcattccaatggggtgactttg

SEQ ID NO: 200 - DNA, 24 bp, Artificial sequence
  Exemplary element X variant
ttttcattccaatggggtgacttt

SEQ ID NO: 201 - DNA, 25 bp, Artificial sequence
  Exemplary element X variant
cttttcattccaatggggtgacttt

SEQ ID NO: 202 - DNA, 24 bp, Artificial sequence
  Exemplary element X variant
ttttcattccaatgactttgcttc

SEQ ID NO: 203 - DNA, 23 bp, Artificial sequence
  Exemplary element X variant
tttcattccaatggggtgacttt

SEQ ID NO: 204 - DNA, 19 bp, Artificial sequence
  Exemplary element X variant
ttttcattcgggtgacttt

SEQ ID NO: 205 - DNA, 19 bp, Artificial sequence
  Exemplary element X variant
tttcattcgggtgactttg

SEQ ID NO: 206 - DNA, 19 bp, Artificial sequence
  Exemplary element X variant
attccaatggggtgacttt

SEQ ID NO: 207 - DNA, 19 bp, Artificial sequence
  Exemplary element X variant
ttccaatggggtgactttg

SEQ ID NO: 208 - DNA, 24 bp, Artificial sequence
  Exemplary element X variant
ttttcattccaatggggtgacttt

SEQ ID NO: 209 - DNA, 24 bp, Artificial sequence
  Exemplary element X variant
tttcattccaatggggtgactttg

SEQ ID NO: 210 - DNA, 24 bp, Artificial sequence
  Exemplary element X variant
tttcattccaatggggtgactttg

SEQ ID NO: 211 - DNA, 24 bp, Artificial sequence
  Exemplary element X variant
ttttcattccaatggggtgacttt

SEQ ID NO: 212 - DNA, 24 bp, Artificial sequence
  Exemplary element X variant
cttttcattccaatggggtgactt

SEQ ID NO: 213 - DNA, 24 bp, Artificial sequence
  Exemplary element X variant
acttttcattccaatggggtgact

SEQ ID NO: 214 - DNA, 24 bp, Artificial sequence
  Exemplary element X variant
ttccaatggggtgactttgcttct

SEQ ID NO: 215 - DNA, 24 bp, Artificial sequence
  Exemplary element X variant
attccaatggggtgactttgcttc

SEQ ID NO: 216 - DNA, 24 bp, Artificial sequence
  Exemplary element X variant
cattccaatggggtgactttgctt

SEQ ID NO: 217 - DNA, 24 bp, Artificial sequence
  Exemplary element X variant
tcattccaatggggtgactttgct

SEQ ID NO: 218 - DNA, 24 bp, Artificial sequence
  Exemplary element X variant
ttcattccaatggggtgactttgc

SEQ ID NO: 219 - DNA, 24 bp, Artificial sequence
  Exemplary element X variant
tttcattccaatggggtgactttg

SEQ ID NO: 220 - DNA, 24 bp, Artificial sequence
  Exemplary element X variant
ttttcattccaatggggtgacttt

SEQ ID NO: 221 - DNA, 24 bp, Artificial sequence
  Exemplary element X variant
cttttcattccaatggggtgactt

SEQ ID NO: 222 - DNA, 24 bp, Artificial sequence
  Exemplary element X variant
acttttcattccaatggggtgact

SEQ ID NO: 223 - DNA, 29 bp, Artificial sequence
  Exemplary element X variant
tttcattccaatggggtgactttgcttct

SEQ ID NO: 224 - DNA, 29 bp, Artificial sequence
  Exemplary element X variant
ttttcattccaatggggtgactttgcttc

SEQ ID NO: 225 - DNA, 29 bp, Artificial sequence
  Exemplary element X variant
cttttcattccaatggggtgactttgctt

SEQ ID NO: 226 - DNA, 29 bp, Artificial sequence
  Exemplary element X variant
acttttcattccaatggggtgactttgct

SEQ ID NO: 227 - DNA, 29 bp, Artificial sequence
  Exemplary element X variant
tacttttcattccaatggggtgactttgc

SEQ ID NO: 228 - DNA, 29 bp, Artificial sequence
  Exemplary element X variant
atacttttcattccaatggggtgactttg

SEQ ID NO: 229 - DNA, 29 bp, Artificial sequence
  Exemplary element X variant
catacttttcattccaatggggtgacttt

SEQ ID NO: 230 - DNA, 29 bp, Artificial sequence
  Exemplary element X variant
tcatacttttcattccaatggggtgactt

SEQ ID NO: 231 - DNA, 29 bp, Artificial sequence
  Exemplary element X variant
ttcatacttttcattccaatggggtgact

SEQ ID NO: 232 - DNA, 26 bp, Artificial sequence
  Exemplary element X variant
tttcattccaatggggtgactttgct

SEQ ID NO: 233 - DNA, 24 bp, Artificial sequence
  Exemplary element X variant
cttttcattccaatgactttgctt

SEQ ID NO: 234 - DNA, 24 bp, Artificial sequence
  Exemplary element X variant
cttttcattcgggtgactttgctt

SEQ ID NO: 235 - DNA, 24 bp, Artificial sequence
  Exemplary element X variant
ttttcattccaatgactttgcttc

SEQ ID NO: 236 - DNA, 24 bp, Artificial sequence
  Exemplary element X variant
ttttcattcgggtgactttgcttc

SEQ ID NO: 237 - DNA, 24 bp, Artificial sequence
  Exemplary element X variant
attccaatggggtgactttgcttc

SEQ ID NO: 238 - DNA, 24 bp, Artificial sequence
  Exemplary element X variant
ttccaatggggtgactttgcttct

SEQ ID NO: 239 - DNA, 24 bp, Artificial sequence
  Exemplary element X variant
cattccaatggggtgactttgctt

SEQ ID NO: 240 - DNA, 24 bp, Artificial sequence
  Exemplary element X variant
tcattccaatggggtgactttgct

SEQ ID NO: 241 - DNA, 29 bp, Artificial sequence
  Exemplary element X variant
tttcattccaatggggtgactttgcttct

SEQ ID NO: 242 - DNA, 29 bp, Artificial sequence
  Exemplary element X variant
ttttcattccaatggggtgactttgcttc

SEQ ID NO: 243 - DNA, 29 bp, Artificial sequence
  Exemplary element X variant
cttttcattccaatggggtgactttgctt

SEQ ID NO: 244 - DNA, 29 bp, Artificial sequence
  Exemplary element X variant
tcatacttttcattccaatgactttgctt

SEQ ID NO: 245 - DNA, 29 bp, Artificial sequence
  Exemplary element X variant
catacttttcattccaatgactttgcttc

SEQ ID NO: 246 - DNA, 29 bp, Artificial sequence
  Exemplary element X variant
atacttttcattccaatgactttgcttct

SEQ ID NO: 247 - DNA, 29 bp, Artificial sequence
  Exemplary element X variant
tacttttcattccaatgactttgcttctg

SEQ ID NO: 248 - DNA, 29 bp, Artificial sequence
  Exemplary element X variant
acttttcattccaatgactttgcttctgg

SEQ ID NO: 249 - DNA, 29 bp, Artificial sequence
  Exemplary element X variant
tttcattccaatggggtgactttgcttct

SEQ ID NO: 250 - DNA, 29 bp, Artificial sequence
  Exemplary element X variant
ttttcattccaatggggtgactttgcttc

SEQ ID NO: 251 - DNA, 29 bp, Artificial sequence
  Exemplary element X variant
cttttcattccaatggggtgactttgctt

SEQ ID NO: 252 - DNA, 29 bp, Artificial sequence
  Exemplary element X variant
acttttcattccaatggggtgactttgct

SEQ ID NO: 253 - DNA, 29 bp, Artificial sequence
  Exemplary element X variant
ttcatacttttcattcgggtgactttgct

SEQ ID NO: 254 - DNA, 29 bp, Artificial sequence
  Exemplary element X variant
tcatacttttcattcgggtgactttgctt

SEQ ID NO: 255 - DNA, 29 bp, Artificial sequence
  Exemplary element X variant
catacttttcattcgggtgactttgcttc

SEQ ID NO: 256 - DNA, 29 bp, Artificial sequence
  Exemplary element X variant
atacttttcattcgggtgactttgcttct

SEQ ID NO: 257 - DNA, 29 bp, Artificial sequence
  Exemplary element X variant
tacttttcattcgggtgactttgcttctg

SEQ ID NO: 258 - DNA, 29 bp, Artificial sequence
  Exemplary element X variant
acttttcattcgggtgactttgcttctgg

SEQ ID NO: 259 - DNA, 29 bp, Artificial sequence
  Exemplary element X variant
acttttcattccaatggggtgactttgct

SEQ ID NO: 260 - DNA, 29 bp, Artificial sequence
  Exemplary element X variant
cttttcattccaatggggtgactttgctt

SEQ ID NO: 261 - DNA, 29 bp, Artificial sequence
  Exemplary element X variant
ttttcattccaatggggtgactttgcttc

SEQ ID NO: 262 - DNA, 29 bp, Artificial sequence
  Exemplary element X variant
tttcattccaatggggtgactttgcttct

SEQ ID NO: 263 - DNA, 29 bp, Artificial sequence
  Exemplary element X variant
ttcattccaatggggtgactttgcttctg

SEQ ID NO: 264 - DNA, 29 bp, Artificial sequence
  Exemplary element X variant
tcattccaatggggtgactttgcttctgg

SEQ ID NO: 265 - DNA, 34 bp, Artificial sequence
  Exemplary element X variant
ttcatacttttcattccaatggggtgactttgct

SEQ ID NO: 266 - DNA, 34 bp, Artificial sequence
  Exemplary element X variant
tcatacttttcattccaatggggtgactttgctt

SEQ ID NO: 267 - DNA, 34 bp, Artificial sequence
  Exemplary element X variant
catacttttcattccaatggggtgactttgcttc

SEQ ID NO: 268 - DNA, 34 bp, Artificial sequence
  Exemplary element X variant
atacttttcattccaatggggtgactttgcttct

SEQ ID NO: 269 - DNA, 34 bp, Artificial sequence
  Exemplary element X variant
tacttttcattccaatggggtgactttgcttctg

SEQ ID NO: 270 - DNA, 34 bp, Artificial sequence
  Exemplary element X variant
acttttcattccaatggggtgactttgcttctgg

SEQ ID NO: 271 - DNA, 28 bp, Artificial sequence
  Exemplary element X variant
tttcattccaatggggtgactttgcttc

SEQ ID NO: 272 - DNA, 24 bp, Artificial sequence
  Exemplary element X variant
ttttcattcgggtgactttgcttc

SEQ ID NO: 273 - DNA, 24 bp, Artificial sequence
  Exemplary element X variant
ttttcattccaatgactttgcttc

SEQ ID NO: 274 - DNA, 24 bp, Artificial sequence
  Exemplary element X variant
ttttcattccaatggggtggcttc

SEQ ID NO: 275 - DNA, 25 bp, Artificial sequence
  Exemplary element X variant
cttttcattccaatggggtggcttc

SEQ ID NO: 276 - DNA, 24 bp, Artificial sequence
  Exemplary element X variant
catacttttcattcgggtggcttc

SEQ ID NO: 277 - DNA, 24 bp, Artificial sequence
  Exemplary element X variant
catacttttcattcactttgcttc

SEQ ID NO: 278 - DNA, 24 bp, Artificial sequence
  Exemplary element X variant
catacttttcattccaatggcttc

SEQ ID NO: 279 - DNA, 24 bp, Artificial sequence
  Exemplary element X variant
tacttcatacttttcattcgcttc

SEQ ID NO: 280 - DNA, 23 bp, Artificial sequence
  Exemplary element X variant
ttccaatggggtgactttgcttc

SEQ ID NO: 281 - DNA, 19 bp, Artificial sequence
  Exemplary element X variant
ttccaatgactttgcttct

SEQ ID NO: 282 - DNA, 19 bp, Artificial sequence
  Exemplary element X variant
ttccaatggggtggcttct

SEQ ID NO: 283 - DNA, 19 bp, Artificial sequence
  Exemplary element X variant
tttcattccaatggcttct

SEQ ID NO: 284 - DNA, 19 bp, Artificial sequence
  Exemplary element X variant
ttttcattccaatggcttc

SEQ ID NO: 285 - DNA, 24 bp, Artificial sequence
  Exemplary element X variant
attccaatggggtgactttgcttc

SEQ ID NO: 286 - DNA, 20 bp, Artificial sequence
  Exemplary element X variant
attccaatgactttgcttct

SEQ ID NO: 287 - DNA, 20 bp, Artificial sequence
  Exemplary element X variant
attccaatggggtggcttct

SEQ ID NO: 288 - DNA, 28 bp, Artificial sequence
  Exemplary element X variant
tttcattccaatggggtgactttgcttc

SEQ ID NO: 289 - DNA, 24 bp, Artificial sequence
  Exemplary element X variant
ttttcattccaatgactttgcttc

SEQ ID NO: 290 - DNA, 24 bp, Artificial sequence
  Exemplary element X variant
ttttcattccaatggggtggcttc

SEQ ID NO: 291 - DNA, 24 bp, Artificial sequence
  Exemplary element X variant
catacttttcattccaatggcttc

SEQ ID NO: 292 - DNA, 28 bp, Artificial sequence
  Exemplary element X variant
tttcattccaatggggtgactttgcttc

SEQ ID NO: 293 - DNA, 24 bp, Artificial sequence
  Exemplary element X variant
ttttcattccaatgactttgcttc

SEQ ID NO: 294 - DNA, 28 bp, Artificial sequence
  Exemplary element X variant
tttcattccaatggggtgactttgcttc

SEQ ID NO: 295 - DNA, 23 bp, Artificial sequence
  Exemplary element X variant
tttcattccaatggggtggcttc

SEQ ID NO: 296 - DNA, 23 bp, Artificial sequence
  Exemplary element X variant
tttcattcgggtgactttgcttc

SEQ ID NO: 297 - DNA, 19 bp, Artificial sequence
  Exemplary element X variant
tttcattcgggtggcttct

SEQ ID NO: 298 - DNA, 19 bp, Artificial sequence
  Exemplary element X variant
ttttcattcgggtggcttc

SEQ ID NO: 299 - DNA, 23 bp, Artificial sequence
  Exemplary element X variant
ttccaatggggtgactttgcttc

SEQ ID NO: 300 - DNA, 19 bp, Artificial sequence
  Exemplary element X variant
attccaatggggtggcttc

SEQ ID NO: 301 - DNA, 19 bp, Artificial sequence
  Exemplary element X variant
attcaatggggtggcttct

SEQ ID NO: 302 - DNA, 28 bp, Artificial sequence
  Exemplary element X variant
tttcattccaatggggtgactttgcttc

SEQ ID NO: 303 - DNA, 24 bp, Artificial sequence
  Exemplary element X variant
ttttcattccaatggggtggcttc

SEQ ID NO: 304 - DNA, 28 bp, Artificial sequence
  Exemplary element X variant
tttcattccaatggggtgactttgcttc

SEQ ID NO: 305 - DNA, 23 bp, Artificial sequence
  Exemplary element X variant
tttcattcgggtgactttgcttc

SEQ ID NO: 306 - DNA, 23 bp, Artificial sequence
  Exemplary element X variant
tttcattccaatgactttgcttc

SEQ ID NO: 307 - DNA, 19 bp, Artificial sequence
  Exemplary element X variant
ttttcattcactttgcttc

SEQ ID NO: 308 - DNA, 19 bp, Artificial sequence
  Exemplary element X variant
tttcattcactttgcttct

SEQ ID NO: 309 - DNA, 23 bp, Artificial sequence
  Exemplary element X variant
ttccaatggggtgactttgcttc

SEQ ID NO: 310 - DNA, 19 bp, Artificial sequence
  Exemplary element X variant
ttccaatgactttgcttct

SEQ ID NO: 311 - DNA, 19 bp, Artificial sequence
  Exemplary element X variant
attccaatgactttgcttc

SEQ ID NO: 312 - DNA, 28 bp, Artificial sequence
  Exemplary element X variant
tttcattccaatggggtgactttgcttc

SEQ ID NO: 313 - DNA, 24 bp, Artificial sequence
  Exemplary element X variant
ttttcattccaatgactttgcttc

SEQ ID NO: 314 - DNA, 24 bp, Artificial sequence
  Exemplary element X variant
tttcattccaatgactttgcttct

SEQ ID NO: 315 - DNA, 28 bp, Artificial sequence
  Exemplary element X variant
tttcattccaatggggtgactttgcttc

SEQ ID NO: 316 - DNA, 23 bp, Artificial sequence
  Exemplary element X variant
tttcattcgggtgactttgcttc

SEQ ID NO: 317 - DNA, 23 bp, Artificial sequence
  Exemplary element X variant
ttccaatggggtgactttgcttc

SEQ ID NO: 318 - DNA, 28 bp, Artificial sequence
  Exemplary element X variant
tttcattccaatggggtgactttgcttc

SEQ ID NO: 319 - DNA, 28 bp, Artificial sequence
  Exemplary element X variant
tttcattccaatggggtgactttgcttc

SEQ ID NO: 320 - DNA, 24 bp, Artificial sequence
  Exemplary element X variant
ttttcattcgggtgactttgcttc

SEQ ID NO: 321 - DNA, 24 bp, Artificial sequence
  Exemplary element X variant
ttttcattccaatggggtggcttc

SEQ ID NO: 322 - DNA, 24 bp, Artificial sequence
  Exemplary element X variant
catacttttcattcgggtggcttc

SEQ ID NO: 323 - DNA, 24 bp, Artificial sequence
  Exemplary element X variant
tttcattcgggtgactttgcttct

SEQ ID NO: 324 - DNA, 24 bp, Artificial sequence
  Exemplary element X variant
tttcattccaatggggtggcttct

SEQ ID NO: 325 - DNA, 24 bp, Artificial sequence
  Exemplary element X variant
atacttttcattcgggtggcttct

SEQ ID NO: 326 - DNA, 24 bp, Artificial sequence
  Exemplary element X variant
tacttttcattcgggtggcttctg

SEQ ID NO: 327 - DNA, 24 bp, Artificial sequence
  Exemplary element X variant
acttttcattcgggtggcttctgg

SEQ ID NO: 328 - DNA, 24 bp, Artificial sequence
  Exemplary element X variant
ttccaatggggtgactttgcttct

SEQ ID NO: 329 - DNA, 24 bp, Artificial sequence
  Exemplary element X variant
attccaatggggtgactttgcttc

SEQ ID NO: 330 - DNA, 24 bp, Artificial sequence
  Exemplary element X variant
ttttcattccaatggggtggcttc

SEQ ID NO: 331 - DNA, 24 bp, Artificial sequence
  Exemplary element X variant
tttcattccaatggggtggcttct

SEQ ID NO: 332 - DNA, 24 bp, Artificial sequence
  Exemplary element X variant
ttcattccaatggggtggcttctg

SEQ ID NO: 333 - DNA, 29 bp, Artificial sequence
  Exemplary element X variant
tcattccaatggggtgactttgcttctgg

SEQ ID NO: 334 - DNA, 28 bp, Artificial sequence
  Exemplary element X variant
tttcattccaatggggtgactttgcttc

SEQ ID NO: 335 - DNA, 30 bp, Artificial sequence
  Exemplary element X variant
ttttcattccaatggggtgactttgcttct

SEQ ID NO: 336 - DNA, 29 bp, Artificial sequence
  Exemplary element X variant
atacttttcattccaatggggtggcttct

SEQ ID NO: 337 - DNA, 29 bp, Artificial sequence
  Exemplary element X variant
catacttttcattccaatggggtggcttc

SEQ ID NO: 338 - DNA, 28 bp, Artificial sequence
  Exemplary element X variant
tttcattccaatggggtgactttgcttc

SEQ ID NO: 339 - DNA, 24 bp, Artificial sequence
  Exemplary element X variant
ttttcattccaatgactttgcttc

SEQ ID NO: 340 - DNA, 24 bp, Artificial sequence
  Exemplary element X variant
ttttcattcgggtgactttgcttc

SEQ ID NO: 341 - DNA, 24 bp, Artificial sequence
  Exemplary element X variant
catacttttcattcactttgcttc

SEQ ID NO: 342 - DNA, 24 bp, Artificial sequence
  Exemplary element X variant
atacttttcattcactttgcttct

SEQ ID NO: 343 - DNA, 24 bp, Artificial sequence
  Exemplary element X variant
tacttttcattcactttgcttctg

SEQ ID NO: 344 - DNA, 24 bp, Artificial sequence
  Exemplary element X variant
acttttcattcactttgcttctgg

SEQ ID NO: 345 - DNA, 24 bp, Artificial sequence
  Exemplary element X variant
ttccaatggggtgactttgcttct

SEQ ID NO: 346 - DNA, 24 bp, Artificial sequence
  Exemplary element X variant
attccaatggggtgactttgcttc

SEQ ID NO: 347 - DNA, 24 bp, Artificial sequence
  Exemplary element X variant
tttcattccaatgactttgcttct

SEQ ID NO: 348 - DNA, 24 bp, Artificial sequence
  Exemplary element X variant
ttttcattccaatgactttgcttc

SEQ ID NO: 349 - DNA, 29 bp, Artificial sequence
  Exemplary element X variant
ttttcattccaatggggtgactttgcttc

SEQ ID NO: 350 - DNA, 29 bp, Artificial sequence
  Exemplary element X variant
tttcattccaatggggtgactttgcttct

SEQ ID NO: 351 - DNA, 29 bp, Artificial sequence
  Exemplary element X variant
catacttttcattccaatgactttgcttc

SEQ ID NO: 352 - DNA, 29 bp, Artificial sequence
  Exemplary element X variant
atacttttcattccaatgactttgcttct

SEQ ID NO: 353 - DNA, 29 bp, Artificial sequence
  Exemplary element X variant
tacttttcattccaatgactttgcttctg

SEQ ID NO: 354 - DNA, 29 bp, Artificial sequence
  Exemplary element X variant
acttttcattccaatgactttgcttctgg

SEQ ID NO: 355 - DNA, 29 bp, Artificial sequence
  Exemplary element X variant
ttttcattccaatggggtgactttgcttc

SEQ ID NO: 356 - DNA, 29 bp, Artificial sequence
  Exemplary element X variant
ttttcattccaatggggtgactttgcttc

SEQ ID NO: 357 - DNA, 24 bp, Artificial sequence
  Exemplary element X variant
tttcattcgggtgactttgcttct

SEQ ID NO: 358 - DNA, 29 bp, Artificial sequence
  Exemplary element X variant
catacttttcattcgggtgactttgcttc

SEQ ID NO: 359 - DNA, 29 bp, Artificial sequence
  Exemplary element X variant
atacttttcattcgggtgactttgcttct

SEQ ID NO: 360 - DNA, 29 bp, Artificial sequence
  Exemplary element X variant
tacttttcattcgggtgactttgcttctg

SEQ ID NO: 361 - DNA, 29 bp, Artificial sequence
  Exemplary element X variant
acttttcattcgggtgactttgcttctgg

SEQ ID NO: 362 - DNA, 29 bp, Artificial sequence
  Exemplary element X variant
ttttcattccaatggggtgactttgcttc

SEQ ID NO: 363 - DNA, 29 bp, Artificial sequence
  Exemplary element X variant
tttcattccaatggggtgactttgcttct

SEQ ID NO: 364 - DNA, 29 bp, Artificial sequence
  Exemplary element X variant
ttcattccaatggggtgactttgcttctg

SEQ ID NO: 365 - DNA, 29 bp, Artificial sequence
  Exemplary element X variant
tcattccaatggggtgactttgcttctgg

SEQ ID NO: 366 - DNA, 34 bp, Artificial sequence; identical to SEQ ID NO: 270
  Exemplary element X variant

SEQ ID NO: 367 - DNA, 34 bp, Artificial sequence; identical to SEQ ID NO: 269
  Exemplary element X variant

SEQ ID NO: 368 - DNA, 34 bp, Artificial sequence; identical to SEQ ID NO: 268
  Exemplary element X variant

SEQ ID NO: 369 - DNA, 34 bp, Artificial sequence; identical to SEQ ID NO: 267
  Exemplary element X variant

SEQ ID NO: 370 - DNA, 25 bp, Artificial sequence
  Exemplary element X variant
cttttcattccaatggggtgacttt

SEQ ID NO: 371 - DNA, 25 bp, Artificial sequence
  Exemplary element X variant
acttttcattccaatggggtgactt

SEQ ID NO: 372 - DNA, 25 bp, Artificial sequence
  Exemplary element X variant
tacttttcattccaatggggtgact

SEQ ID NO: 373 - DNA, 25 bp, Artificial sequence
  Exemplary element X variant
atacttttcattccaatggggtgac

SEQ ID NO: 374 - DNA, 25 bp, Artificial sequence
  Exemplary element X variant
catacttttcattccaatggggtga

SEQ ID NO: 375 - DNA, 25 bp, Artificial sequence
  Exemplary element X variant
tcatacttttcattccaatggggtg

SEQ ID NO: 376 - DNA, 25 bp, Artificial sequence
  Exemplary element X variant
ttcatacttttcattccaatggggt

SEQ ID NO: 377 - DNA, 25 bp, Artificial sequence
  Exemplary element X variant
cttcatacttttcattccaatgggg

SEQ ID NO: 378 - DNA, 19 bp, Artificial sequence
  Exemplary element X variant
ttcatacttttcattccaa

SEQ ID NO: 379 - DNA, 19 bp, Artificial sequence
  Exemplary element X variant
tcatacttttcattccaat

SEQ ID NO: 380 - DNA, 19 bp, Artificial sequence
  Exemplary element X variant
catacttttcattccaatg

SEQ ID NO: 381 - DNA, 19 bp, Artificial sequence
  Exemplary element X variant
atacttttcattccaatgg

SEQ ID NO: 382 - DNA, 19 bp, Artificial sequence
  Exemplary element X variant
tacttttcattccaatggg

SEQ ID NO: 383 - DNA, 19 bp, Artificial sequence
  Exemplary element X variant
acttttcattccaatgggg

SEQ ID NO: 384 - DNA, 19 bp, Artificial sequence
  Exemplary element X variant
cttttcattccaatggggt

SEQ ID NO: 385 - DNA, 20 bp, Artificial sequence
  Exemplary element X variant
cttttcattccaatggggtg

SEQ ID NO: 386 - DNA, 20 bp, Artificial sequence
  Exemplary element X variant
catacttttcattcgggtga

SEQ ID NO: 387 - DNA, 20 bp, Artificial sequence
  Exemplary element X variant
atacttttcattcgggtgac

SEQ ID NO: 388 - DNA, 20 bp, Artificial sequence
  Exemplary element X variant
tacttttcattcgggtgact

SEQ ID NO: 389 - DNA, 20 bp, Artificial sequence
  Exemplary element X variant
acttttcattcgggtgactt

SEQ ID NO: 390 - DNA, 20 bp, Artificial sequence
  Exemplary element X variant
cttttcattcgggtgacttt

SEQ ID NO: 391 - DNA, 20 bp, Artificial sequence
  Exemplary element X variant
cttttcattccaatggggtg

SEQ ID NO: 392 - DNA, 19 bp, Artificial sequence
  Exemplary element X variant
catacttttcaatggggtg

SEQ ID NO: 393 - DNA, 19 bp, Artificial sequence
  Exemplary element X variant
atacttttcaatggggtga

SEQ ID NO: 394 - DNA, 19 bp, Artificial sequence
  Exemplary element X variant
tacttttcaatggggtgac

SEQ ID NO: 395 - DNA, 19 bp, Artificial sequence
  Exemplary element X variant
acttttcaatggggtgact

SEQ ID NO: 396 - DNA, 19 bp, Artificial sequence
  Exemplary element X variant
cttttcaatggggtgactt

SEQ ID NO: 397 - DNA, 24 bp, Artificial sequence
  Exemplary element X variant
catacttttcattccaatggggtg

SEQ ID NO: 398 - DNA, 24 bp, Artificial sequence
  Exemplary element X variant
atacttttcattccaatggggtga

SEQ ID NO: 399 - DNA, 24 bp, Artificial sequence
  Exemplary element X variant
tacttttcattccaatggggtgac

SEQ ID NO: 400 - DNA, 24 bp, Artificial sequence
  Exemplary element X variant
acttttcattccaatggggtgact

SEQ ID NO: 401 - DNA, 24 bp, Artificial sequence
  Exemplary element X variant
cttttcattccaatggggtgactt

SEQ ID NO: 402 - DNA, 25 bp, Artificial sequence
  Exemplary element X variant
cttttcattccaatggggtgacttt

SEQ ID NO: 403 - DNA, 20 bp, Artificial sequence
  Exemplary element X variant
cttttcattccaatgacttt

SEQ ID NO: 404 - DNA, 20 bp, Artificial sequence
  Exemplary element X variant
cttttcattcgggtgacttt

SEQ ID NO: 405 - DNA, 19 bp, Artificial sequence
  Exemplary element X variant
catacttttcattcacttt

SEQ ID NO: 406 - DNA, 19 bp, Artificial sequence
  Exemplary element X variant
atacttttcattcactttg

SEQ ID NO: 407 - DNA, 19 bp, Artificial sequence
  Exemplary element X variant
tacttttcattcactttgc

SEQ ID NO: 408 - DNA, 19 bp, Artificial sequence
  Exemplary element X variant
acttttcattcactttgct

SEQ ID NO: 409 - DNA, 19 bp, Artificial sequence
  Exemplary element X variant
cttttcattcactttgctt

SEQ ID NO: 410 - DNA, 25 bp, Artificial sequence
  Exemplary element X variant
cttttcattccaatggggtgacttt

SEQ ID NO: 411 - DNA, 20 bp, Artificial sequence
  Exemplary element X variant
cttttcattccaatgacttt

SEQ ID NO: 412 - DNA, 20 bp, Artificial sequence
  Exemplary element X variant
cttttcaatggggtgacttt

SEQ ID NO: 413 - DNA, 19 bp, Artificial sequence
  Exemplary element X variant
catacttttcaatgacttt

SEQ ID NO: 414 - DNA, 19 bp, Artificial sequence
  Exemplary element X variant
atacttttcaatgactttg

SEQ ID NO: 415 - DNA, 19 bp, Artificial sequence
  Exemplary element X variant
tacttttcaatgactttgc

SEQ ID NO: 416 - DNA, 19 bp, Artificial sequence
  Exemplary element X variant
acttttcaatgactttgct

SEQ ID NO: 417 - DNA, 19 bp, Artificial sequence
  Exemplary element X variant
cttttcaatgactttgctt

SEQ ID NO: 418 - DNA, 25 bp, Artificial sequence
  Exemplary element X variant
cttttcattccaatggggtgacttt

SEQ ID NO: 419 - DNA, 24 bp, Artificial sequence
  Exemplary element X variant
catacttttcattccaatgacttt

SEQ ID NO: 420 - DNA, 24 bp, Artificial sequence
  Exemplary element X variant
atacttttcattccaatgactttg

SEQ ID NO: 421 - DNA, 24 bp, Artificial sequence
  Exemplary element X variant
tacttttcattccaatgactttgc

SEQ ID NO: 422 - DNA, 24 bp, Artificial sequence
  Exemplary element X variant
acttttcattccaatgactttgct

SEQ ID NO: 423 - DNA, 24 bp, Artificial sequence
  Exemplary element X variant
cttttcattccaatgactttgctt

SEQ ID NO: 424 - DNA, 25 bp, Artificial sequence
  Exemplary element X variant
cttttcattccaatggggtgacttt

SEQ ID NO: 425 - DNA, 20 bp, Artificial sequence
  Exemplary element X variant
cttttcattcgggtgacttt

SEQ ID NO: 426 - DNA, 25 bp, Artificial sequence
  Exemplary element X variant
cttttcattccaatggggtgacttt

SEQ ID NO: 427 - DNA, 24 bp, Artificial sequence
  Exemplary element X variant
cttttcattccaatggggtgactt

SEQ ID NO: 428 - DNA, 24 bp, Artificial sequence
  Exemplary element X variant
acttttcattccaatggggtgact

SEQ ID NO: 429 - DNA, 24 bp, Artificial sequence
  Exemplary element X variant
ttcatacttttcattcgggtgact

SEQ ID NO: 430 - DNA, 24 bp, Artificial sequence
  Exemplary element X variant
tcatacttttcattcgggtgactt

SEQ ID NO: 431 - DNA, 24 bp, Artificial sequence
  Exemplary element X variant
catacttttcattcgggtgacttt

SEQ ID NO: 432 - DNA, 24 bp, Artificial sequence
  Exemplary element X variant
atacttttcattcgggtgactttg

SEQ ID NO: 433 - DNA, 24 bp, Artificial sequence
  Exemplary element X variant
tacttttcattcgggtgactttgc

SEQ ID NO: 434 - DNA, 24 bp, Artificial sequence
  Exemplary element X variant
acttttcattcgggtgactttgct

SEQ ID NO: 435 - DNA, 24 bp, Artificial sequence
  Exemplary element X variant
cttttcattcgggtgactttgctt

SEQ ID NO: 436 - DNA, 24 bp, Artificial sequence
  Exemplary element X variant
cttttcattccaatggggtgactt

SEQ ID NO: 437 - DNA, 24 bp, Artificial sequence
  Exemplary element X variant
acttttcattccaatggggtgact

SEQ ID NO: 438 - DNA, 24 bp, Artificial sequence
  Exemplary element X variant
ttcatacttttcaatggggtgact

SEQ ID NO: 439 - DNA, 24 bp, Artificial sequence
  Exemplary element X variant
tcatacttttcaatggggtgactt

SEQ ID NO: 440 - DNA, 24 bp, Artificial sequence
  Exemplary element X variant
catacttttcaatggggtgacttt

SEQ ID NO: 441 - DNA, 24 bp, Artificial sequence
  Exemplary element X variant
atacttttcaatggggtgactttg

SEQ ID NO: 442 - DNA, 24 bp, Artificial sequence
  Exemplary element X variant
tacttttcaatggggtgactttgc

SEQ ID NO: 443 - DNA, 24 bp, Artificial sequence
  Exemplary element X variant
acttttcaatggggtgactttgct

SEQ ID NO: 444 - DNA, 24 bp, Artificial sequence
  Exemplary element X variant
cttttcaatggggtgactttgctt

SEQ ID NO: 445 - DNA, 29 bp, Artificial sequence
  Exemplary element X variant
ttcatacttttcattccaatggggtgact

SEQ ID NO: 446 - DNA, 29 bp, Artificial sequence
  Exemplary element X variant
tcatacttttcattccaatggggtgactt

SEQ ID NO: 447 - DNA, 29 bp, Artificial sequence
  Exemplary element X variant
catacttttcattccaatggggtgacttt

SEQ ID NO: 448 - DNA, 29 bp, Artificial sequence
  Exemplary element X variant
atacttttcattccaatggggtgactttg

SEQ ID NO: 449 - DNA, 29 bp, Artificial sequence
  Exemplary element X variant
tacttttcattccaatggggtgactttgc

SEQ ID NO: 450 - DNA, 29 bp, Artificial sequence
  Exemplary element X variant
acttttcattccaatggggtgactttgct

SEQ ID NO: 451 - DNA, 29 bp, Artificial sequence
  Exemplary element X variant
cttttcattccaatggggtgactttgctt

SEQ ID NO: 452 - DNA, 28 bp, Artificial sequence
  Exemplary element X variant
cttttcattccaatggggtgactttgct

SEQ ID NO: 453 - DNA, 24 bp, Artificial sequence
  Exemplary element X variant
cttttcattcgggtgactttgctt

SEQ ID NO: 454 - DNA, 24 bp, Artificial sequence
  Exemplary element X variant
cttttcattccaatgactttgctt

SEQ ID NO: 455 - DNA, 24 bp, Artificial sequence
  Exemplary element X variant
tcatacttttcattcactttgctt

SEQ ID NO: 456 - DNA, 24 bp, Artificial sequence
  Exemplary element X variant
acttttcattcgggtgactttgct

SEQ ID NO: 457 - DNA, 24 bp, Artificial sequence
  Exemplary element X variant
acttttcattccaatgactttgct

SEQ ID NO: 458 - DNA, 24 bp, Artificial sequence
  Exemplary element X variant
ttcatacttttcattcactttgct

SEQ ID NO: 459 - DNA, 28 bp, Artificial sequence
  Exemplary element X variant
cttttcattccaatggggtgactttgct

SEQ ID NO: 460 - DNA, 24 bp, Artificial sequence
  Exemplary element X variant
cttttcaatggggtgactttgctt

SEQ ID NO: 461 - DNA, 24 bp, Artificial sequence
  Exemplary element X variant
cttttcattccaatgactttgctt

SEQ ID NO: 462 - DNA, 24 bp, Artificial sequence
  Exemplary element X variant
tcatacttttcaatgactttgctt

SEQ ID NO: 463 - DNA, 24 bp, Artificial sequence
  Exemplary element X variant
acttttcaatggggtgactttgct

SEQ ID NO: 464 - DNA, 24 bp, Artificial sequence
  Exemplary element X variant
acttttcattccaatgactttgct

SEQ ID NO: 465 - DNA, 24 bp, Artificial sequence
  Exemplary element X variant
ttcatacttttcaatgactttgct

SEQ ID NO: 466 - DNA, 29 bp, Artificial sequence
  Exemplary element X variant
cttttcattccaatggggtgactttgctt

SEQ ID NO: 467 - DNA, 29 bp, Artificial sequence
  Exemplary element X variant
acttttcattccaatggggtgactttgct

SEQ ID NO: 468 - DNA, 29 bp, Artificial sequence
  Exemplary element X variant
acttttcattccaatgactttgcttctgg

SEQ ID NO: 469 - DNA, 29 bp, Artificial sequence
  Exemplary element X variant
cttttcattccaatgactttgcttctgga

SEQ ID NO: 470 - DNA, 29 bp, Artificial sequence
  Exemplary element X variant
tcatacttttcattccaatgactttgctt

SEQ ID NO: 471 - DNA, 29 bp, Artificial sequence
  Exemplary element X variant
ttcatacttttcattccaatgactttgct

SEQ ID NO: 472 - DNA, 29 bp, Artificial sequence
  Exemplary element X variant
cttttcattccaatggggtgactttgctt

SEQ ID NO: 473 - DNA, 29 bp, Artificial sequence
  Exemplary element X variant
acttttcattccaatggggtgactttgct

SEQ ID NO: 474 - DNA, 29 bp, Artificial sequence
  Exemplary element X variant
acttttcaatggggtgactttgcttctgg

SEQ ID NO: 475 - DNA, 29 bp, Artificial sequence
  Exemplary element X variant
cttttcaatggggtgactttgcttctgga

SEQ ID NO: 476 - DNA, 29 bp, Artificial sequence
  Exemplary element X variant
tcatacttttcaatggggtgactttgctt

SEQ ID NO: 477 - DNA, 29 bp, Artificial sequence
  Exemplary element X variant
ttcatacttttcaatggggtgactttgct

SEQ ID NO: 478 - DNA, 34 bp, Artificial sequence; identical to SEQ ID NO: 270
  Exemplary element X variant

SEQ ID NO: 479 - DNA, 34 bp, Artificial sequence; identical to SEQ ID NO: 269
  Exemplary element X variant

SEQ ID NO: 480 - DNA, 34 bp, Artificial sequence; identical to SEQ ID NO: 268
  Exemplary element X variant

SEQ ID NO: 481 - DNA, 34 bp, Artificial sequence; identical to SEQ ID NO: 267
  Exemplary element X variant

SEQ ID NO: 482 - DNA, 34 bp, Artificial sequence; identical to SEQ ID NO: 266
  Exemplary element X variant

SEQ ID NO: 483 - DNA, 34 bp, Artificial sequence; identical to SEQ ID NO: 265
  Exemplary element X variant

SEQ ID NO: 484 - DNA, 30 bp, Artificial sequence
  Exemplary element X variant
cttttcattccaatggggtgactttgcttc

SEQ ID NO: 485 - DNA, 25 bp, Artificial sequence
  Exemplary element X variant
cttttcattcgggtgactttgcttc

SEQ ID NO: 486 - DNA, 25 bp, Artificial sequence
  Exemplary element X variant
cttttcattccaatgactttgcttc

SEQ ID NO: 487 - DNA, 25 bp, Artificial sequence
  Exemplary element X variant
cttttcattccaatggggtggcttc

SEQ ID NO: 488 - DNA, 20 bp, Artificial sequence
  Exemplary element X variant
cttttcattcactttgcttc

SEQ ID NO: 489 - DNA, 20 bp, Artificial sequence
  Exemplary element X variant
cttttcattccaatggcttc

SEQ ID NO: 490 - DNA, 20 bp, Artificial sequence
  Exemplary element X variant
cttttcattcgggtggcttc

SEQ ID NO: 491 - DNA, 19 bp, Artificial sequence
  Exemplary element X variant
cttttcattcgcttctgga

SEQ ID NO: 492 - DNA, 19 bp, Artificial sequence
  Exemplary element X variant
acttttcattcgcttctgg

SEQ ID NO: 493 - DNA, 19 bp, Artificial sequence
  Exemplary element X variant
tacttttcattcgcttctg

SEQ ID NO: 494 - DNA, 19 bp, Artificial sequence
  Exemplary element X variant
atacttttcattcgcttct

SEQ ID NO: 495 - DNA, 19 bp, Artificial sequence
  Exemplary element X variant
catacttttcattcgcttc

SEQ ID NO: 496 - DNA, 25 bp, Artificial sequence
  Exemplary element X variant
cttttcaatggggtgactttgcttc

SEQ ID NO: 497 - DNA, 25 bp, Artificial sequence
  Exemplary element X variant
cttttcattccaatgactttgcttc

SEQ ID NO: 498 - DNA, 25 bp, Artificial sequence
  Exemplary element X variant
cttttcattccaatggggtggcttc

SEQ ID NO: 499 - DNA, 20 bp, Artificial sequence
  Exemplary element X variant
cttttcaatgactttgcttc

SEQ ID NO: 500 - DNA, 20 bp, Artificial sequence
  Exemplary element X variant
cttttcaatggggtggcttc

SEQ ID NO: 501 - DNA, 20 bp, Artificial sequence
  Exemplary element X variant
cttttcattccaatggcttc

SEQ ID NO: 502 - DNA, 30 bp, Artificial sequence; identical to SEQ ID NO: 484
  Exemplary element X variant

SEQ ID NO: 503 - DNA, 19 bp, Artificial sequence
  Exemplary element X variant
cttttcaatggcttctgga

SEQ ID NO: 504 - DNA, 19 bp, Artificial sequence
  Exemplary element X variant
acttttcaatggcttctgg

SEQ ID NO: 505 - DNA, 19 bp, Artificial sequence
  Exemplary element X variant
tacttttcaatggcttctg

SEQ ID NO: 506 - DNA, 19 bp, Artificial sequence
  Exemplary element X variant
atacttttcaatggcttct

SEQ ID NO: 507 - DNA, 19 bp, Artificial sequence
  Exemplary element X variant
catacttttcaatggcttc

SEQ ID NO: 508 - DNA, 30 bp, Artificial sequence; identical to SEQ ID NO: 484
  Exemplary element X variant

SEQ ID NO: 509 - DNA, 25 bp, Artificial sequence
  Exemplary element X variant
cttttcattccaatgactttgcttc

SEQ ID NO: 510 - DNA, 25 bp, Artificial sequence
  Exemplary element X variant
cttttcattccaatggggtggcttc

SEQ ID NO: 511 - DNA, 24 bp, Artificial sequence
  Exemplary element X variant
cttttcattccaatggcttctgga

SEQ ID NO: 512 - DNA, 24 bp, Artificial sequence
  Exemplary element X variant
acttttcattccaatggcttctgg

SEQ ID NO: 513 - DNA, 24 bp, Artificial sequence
  Exemplary element X variant
tacttttcattccaatggcttctg

SEQ ID NO: 514 - DNA, 24 bp, Artificial sequence
  Exemplary element X variant
atacttttcattccaatggcttct

SEQ ID NO: 515 - DNA, 24 bp, Artificial sequence
  Exemplary element X variant
catacttttcattccaatggcttc

SEQ ID NO: 516 - DNA, 30 bp, Artificial sequence; identical to SEQ ID NO: 484
  Exemplary element X variant

SEQ ID NO: 517 - DNA, 25 bp, Artificial sequence
  Exemplary element X variant
cttttcattcgggtgactttgcttc

SEQ ID NO: 518 - DNA, 25 bp, Artificial sequence
  Exemplary element X variant
cttttcattccaatggggtggcttc

SEQ ID NO: 519 - DNA, 20 bp, Artificial sequence
  Exemplary element X variant
cttttcattcgggtggcttc

SEQ ID NO: 520 - DNA, 30 bp, Artificial sequence; identical to SEQ ID NO: 484
  Exemplary element X variant

SEQ ID NO: 521 - DNA, 25 bp, Artificial sequence
  Exemplary element X variant
cttttcaatggggtgactttgcttc

SEQ ID NO: 522 - DNA, 25 bp, Artificial sequence
  Exemplary element X variant
cttttcattccaatggggtggcttc

SEQ ID NO: 523 - DNA, 20 bp, Artificial sequence
  Exemplary element X variant
cttttcaatggggtggcttc

SEQ ID NO: 524 - DNA, 30 bp, Artificial sequence; identical to SEQ ID NO: 484
  Exemplary element X variant

SEQ ID NO: 525 - DNA, 25 bp, Artificial sequence
  Exemplary element X variant
cttttcattccaatggggtggcttc

SEQ ID NO: 526 - DNA, 30 bp, Artificial sequence; identical to SEQ ID NO: 484
  Exemplary element X variant

SEQ ID NO: 527 - DNA, 25 bp, Artificial sequence
  Exemplary element X variant
cttttcattcgggtgactttgcttc

SEQ ID NO: 528 - DNA, 25 bp, Artificial sequence
  Exemplary element X variant
cttttcattccaatgactttgcttc

SEQ ID NO: 529 - DNA, 20 bp, Artificial sequence
  Exemplary element X variant
cttttcattcactttgcttc

SEQ ID NO: 530 - DNA, 30 bp, Artificial sequence; identical to SEQ ID NO: 484
  Exemplary element X variant

SEQ ID NO: 531 - DNA, 25 bp, Artificial sequence
  Exemplary element X variant
cttttcaatggggtgactttgcttc

SEQ ID NO: 532 - DNA, 25 bp, Artificial sequence
  Exemplary element X variant
cttttcattccaatgactttgcttc

SEQ ID NO: 533 - DNA, 20 bp, Artificial sequence
  Exemplary element X variant
cttttcaatgactttgcttc

SEQ ID NO: 534 - DNA, 30 bp, Artificial sequence; identical to SEQ ID NO: 484
  Exemplary element X variant

SEQ ID NO: 535 - DNA, 25 bp, Artificial sequence
  Exemplary element X variant
cttttcattccaatgactttgcttc

SEQ ID NO: 536 - DNA, 30 bp, Artificial sequence; identical to SEQ ID NO: 484
  Exemplary element X variant

SEQ ID NO: 537 - DNA, 25 bp, Artificial sequence
  Exemplary element X variant
cttttcattcgggtgactttgcttc

SEQ ID NO: 538 - DNA, 30 bp, Artificial sequence; identical to SEQ ID NO: 484
  Exemplary element X variant

SEQ ID NO: 539 - DNA, 25 bp, Artificial sequence
  Exemplary element X variant
cttttcaatggggtgactttgcttc

SEQ ID NO: 540 - DNA, 30 bp, Artificial sequence; identical to SEQ ID NO: 484
  Exemplary element X variant

SEQ ID NO: 541 - DNA, 30 bp, Artificial sequence; identical to SEQ ID NO: 484
  Exemplary element X variant

SEQ ID NO: 542 - DNA, 25 bp, Artificial sequence
  Exemplary element X variant
cttttcattcgggtgactttgcttc

SEQ ID NO: 543 - DNA, 25 bp, Artificial sequence
  Exemplary element X variant
cttttcattccaatggggtggcttc

SEQ ID NO: 544 - DNA, 24 bp, Artificial sequence
  Exemplary element X variant
atacttttcattcgggtggcttct

SEQ ID NO: 545 - DNA, 24 bp, Artificial sequence
  Exemplary element X variant
tacttttcattcgggtggcttctg

SEQ ID NO: 546 - DNA, 24 bp, Artificial sequence
  Exemplary element X variant
acttttcattcgggtggcttctgg

SEQ ID NO: 547 - DNA, 24 bp, Artificial sequence
  Exemplary element X variant
cttttcattcgggtggcttctgga

SEQ ID NO: 548 - DNA, 30 bp, Artificial sequence; identical to SEQ ID NO: 484
  Exemplary element X variant

SEQ ID NO: 549 - DNA, 25 bp, Artificial sequence
  Exemplary element X variant
cttttcaatggggtgactttgcttc

SEQ ID NO: 550 - DNA, 25 bp, Artificial sequence
  Exemplary element X variant
cttttcattccaatggggtggcttc

SEQ ID NO: 551 - DNA, 24 bp, Artificial sequence
  Exemplary element X variant
atacttttcaatggggtggcttct

SEQ ID NO: 552 - DNA, 24 bp, Artificial sequence
  Exemplary element X variant
tacttttcaatggggtggcttctg

SEQ ID NO: 553 - DNA, 24 bp, Artificial sequence
  Exemplary element X variant
acttttcaatggggtggcttctgg

SEQ ID NO: 554 - DNA, 24 bp, Artificial sequence
  Exemplary element X variant
cttttcaatggggtggcttctgga

SEQ ID NO: 555 - DNA, 30 bp, Artificial sequence; identical to SEQ ID NO: 484
  Exemplary element X variant

SEQ ID NO: 556 - DNA, 29 bp, Artificial sequence
  Exemplary element X variant
atacttttcattccaatggggtggcttct

SEQ ID NO: 557 - DNA, 29 bp, Artificial sequence
  Exemplary element X variant
tacttttcattccaatggggtggcttctg

SEQ ID NO: 558 - DNA, 29 bp, Artificial sequence
  Exemplary element X variant
acttttcattccaatggggtggcttctgg

SEQ ID NO: 559 - DNA, 29 bp, Artificial sequence
  Exemplary element X variant
cttttcattccaatggggtggcttctgga

SEQ ID NO: 560 - DNA, 30 bp, Artificial sequence; identical to SEQ ID NO: 484
  Exemplary element X variant

SEQ ID NO: 561 - DNA, 25 bp, Artificial sequence
  Exemplary element X variant
cttttcattcgggtgactttgcttc

SEQ ID NO: 562 - DNA, 25 bp, Artificial sequence
  Exemplary element X variant
cttttcattccaatgactttgcttc

SEQ ID NO: 563 - DNA, 24 bp, Artificial sequence
  Exemplary element X variant
atacttttcattcactttgcttct

SEQ ID NO: 564 - DNA, 24 bp, Artificial sequence
  Exemplary element X variant
tacttttcattcactttgcttctg

SEQ ID NO: 565 - DNA, 24 bp, Artificial sequence
  Exemplary element X variant
acttttcattcactttgcttctgg

SEQ ID NO: 566 - DNA, 24 bp, Artificial sequence
  Exemplary element X variant
cttttcattcactttgcttctgga

SEQ ID NO: 567 - DNA, 30 bp, Artificial sequence; identical to SEQ ID NO: 484
  Exemplary element X variant

SEQ ID NO: 568 - DNA, 25 bp, Artificial sequence
  Exemplary element X variant
cttttcaatggggtgactttgcttc

SEQ ID NO: 569 - DNA, 25 bp, Artificial sequence
  Exemplary element X variant
cttttcattccaatgactttgcttc

SEQ ID NO: 570 - DNA, 24 bp, Artificial sequence
  Exemplary element X variant
atacttttcaatgactttgcttct

SEQ ID NO: 571 - DNA, 24 bp, Artificial sequence
  Exemplary element X variant
tacttttcaatgactttgcttctg

SEQ ID NO: 572 - DNA, 24 bp, Artificial sequence
  Exemplary element X variant
acttttcaatgactttgcttctgg

SEQ ID NO: 573 - DNA, 24 bp, Artificial sequence
  Exemplary element X variant
cttttcaatgactttgcttctgga

SEQ ID NO: 574 - DNA, 30 bp, Artificial sequence; identical to SEQ ID NO: 484
  Exemplary element X variant

SEQ ID NO: 575 - DNA, 29 bp, Artificial sequence
  Exemplary element X variant
atacttttcattccaatgactttgcttct

SEQ ID NO: 576 - DNA, 29 bp, Artificial sequence
  Exemplary element X variant
tacttttcattccaatgactttgcttctg

SEQ ID NO: 577 - DNA, 29 bp, Artificial sequence
  Exemplary element X variant
acttttcattccaatgactttgcttctgg

SEQ ID NO: 578 - DNA, 29 bp, Artificial sequence
  Exemplary element X variant
cttttcattccaatgactttgcttctgga

SEQ ID NO: 579 - DNA, 30 bp, Artificial sequence; identical to SEQ ID NO: 484
  Exemplary element X variant

SEQ ID NO: 580 - DNA, 29 bp, Artificial sequence
  Exemplary element X variant
atacttttcattcgggtgactttgcttct

SEQ ID NO: 581 - DNA, 29 bp, Artificial sequence
  Exemplary element X variant
tacttttcattcgggtgactttgcttctg

SEQ ID NO: 582 - DNA, 29 bp, Artificial sequence
  Exemplary element X variant
acttttcattcgggtgactttgcttctgg

SEQ ID NO: 583 - DNA, 29 bp, Artificial sequence
  Exemplary element X variant
cttttcattcgggtgactttgcttctgga

SEQ ID NO: 584 - DNA, 30 bp, Artificial sequence; identical to SEQ ID NO: 484
  Exemplary element X variant

SEQ ID NO: 585 - DNA, 29 bp, Artificial sequence
  Exemplary element X variant
atacttttcaatggggtgactttgcttct

SEQ ID NO: 586 - DNA, 29 bp, Artificial sequence
  Exemplary element X variant
tacttttcaatggggtgactttgcttctg

SEQ ID NO: 587 - DNA, 29 bp, Artificial sequence
  Exemplary element X variant
acttttcaatggggtgactttgcttctgg

SEQ ID NO: 588 - DNA, 29 bp, Artificial sequence
  Exemplary element X variant
cttttcaatggggtgactttgcttctgga

SEQ ID NO: 589 - DNA, 34 bp, Artificial sequence; identical to SEQ ID NO: 268
  Exemplary element X variant

SEQ ID NO: 590 - DNA, 34 bp, Artificial sequence; identical to SEQ ID NO: 269
  Exemplary element X variant

SEQ ID NO: 591 - DNA, 34 bp, Artificial sequence; identical to SEQ ID NO: 270
  Exemplary element X variant

SEQ ID NO: 592 - DNA, 34 bp, Artificial sequence
  Exemplary element X variant
cttttcattccaatggggtgactttgcttctgga

SEQ ID NO: 593 - DNA, 30 bp, Artificial sequence; identical to SEQ ID NO: 484
  Exemplary element X variant

SEQ ID NO: 594 - DNA, 25 bp, Artificial sequence
  Exemplary element X variant
cttttcaatggggtgactttgcttc

SEQ ID NO: 595 - DNA, 25 bp, Artificial sequence
  Exemplary element X variant
cttttcattcgggtgactttgcttc

SEQ ID NO: 596 - DNA, 24 bp, Artificial sequence
  Exemplary element X variant
cttttgggtgactttgcttctgga

SEQ ID NO: 597 - DNA, 24 bp, Artificial sequence
  Exemplary element X variant
acttttgggtgactttgcttctgg

SEQ ID NO: 598 - DNA, 24 bp, Artificial sequence
  Exemplary element X variant
tacttttgggtgactttgcttctg

SEQ ID NO: 599 - DNA, 24 bp, Artificial sequence
  Exemplary element X variant
atacttttgggtgactttgcttct

SEQ ID NO: 600 - DNA, 24 bp, Artificial sequence
  Exemplary element X variant
catacttttgggtgactttgcttc

SEQ ID NO: 601 - DNA, 29 bp, Artificial sequence
  Exemplary element X variant
gtacttcatagggtgactttgcttctgga

SEQ ID NO: 602 - DNA, 24 bp, Artificial sequence
  Exemplary element X variant
gtacttcataactttgcttctgga

SEQ ID NO: 603 - DNA, 19 bp, Artificial sequence
  Exemplary element X variant
gtacttcatagcttctgga

SEQ ID NO: 604 - DNA, 34 bp, Artificial sequence; identical to SEQ ID NO: 39
  Exemplary element X variant

SEQ ID NO: 605 - DNA, 29 bp, Artificial sequence
  Exemplary element X variant
gtacttcatacttttactttgcttctgga

SEQ ID NO: 606 - DNA, 24 bp, Artificial sequence
  Exemplary element X variant
gtacttcatacttttgcttctgga

SEQ ID NO: 607 - DNA, 34 bp, Artificial sequence
  Exemplary element X variant
gtacttcatacttttcattcactttgcttctgga

SEQ ID NO: 608 - DNA, 29 bp, Artificial sequence
  Exemplary element X variant
gtacttcatacttttcattcgcttctgga

SEQ ID NO: 609 - DNA, 34 bp, Artificial sequence
  Exemplary element X variant
gtacttcatacttttcattccaatggcttctgga

SEQ ID NO: 610 - DNA, 34 bp, Artificial sequence; identical to SEQ ID NO: 40
  Exemplary element X variant

SEQ ID NO: 611 - DNA, 29 bp, Artificial sequence
  Exemplary element X variant
gtactcttttgggtgactttgcttctgga

SEQ ID NO: 612 - DNA, 24 bp, Artificial sequence
  Exemplary element X variant
gtactcttttactttgcttctgga

SEQ ID NO: 613 - DNA, 19 bp, Artificial sequence
  Exemplary element X variant
gtactcttttgcttctgga

SEQ ID NO: 614 - DNA, 34 bp, Artificial sequence; identical to SEQ ID NO: 42
  Exemplary element X variant

SEQ ID NO: 615 - DNA, 29 bp, Artificial sequence
  Exemplary element X variant
gtactcttttcattcactttgcttctgga

SEQ ID NO: 616 - DNA, 24 bp, Artificial sequence
  Exemplary element X variant
gtactcttttcattcgcttctgga

SEQ ID NO: 617 - DNA, 34 bp, Artificial sequence
  Exemplary element X variant
gtactcttttcattccaatgactttgcttctgga

SEQ ID NO: 618 - DNA, 29 bp, Artificial sequence
  Exemplary element X variant
gtactcttttcattccaatggcttctgga

SEQ ID NO: 619 - DNA, 34 bp, Artificial sequence
  Exemplary element X variant
gtactcttttcattccaatggggtggcttctgga

SEQ ID NO: 620 - DNA, 34 bp, Artificial sequence; identical to SEQ ID NO: 41
  Exemplary element X variant

SEQ ID NO: 621 - DNA, 29 bp, Artificial sequence
  Exemplary element X variant
gtacttcatacattcactttgcttctgga

SEQ ID NO: 622 - DNA, 24 bp, Artificial sequence
  Exemplary element X variant
gtacttcatacattcgcttctgga

SEQ ID NO: 623 - DNA, 34 bp, Artificial sequence
  Exemplary element X variant
gtacttcatacattccaatgactttgcttctgga

SEQ ID NO: 624 - DNA, 29 bp, Artificial sequence
  Exemplary element X variant
gtacttcatacattccaatggcttctgga

SEQ ID NO: 625 - DNA, 34 bp, Artificial sequence
  Exemplary element X variant
gtacttcatacattccaatggggtggcttctgga

SEQ ID NO: 626 - DNA, 34 bp, Artificial sequence
  Exemplary element X variant
gtacttcatacttttcaatgactttgcttctgga

SEQ ID NO: 627 - DNA, 29 bp, Artificial sequence
  Exemplary element X variant
gtacttcatacttttcaatggcttctgga

SEQ ID NO: 628 - DNA, 34 bp, Artificial sequence
  Exemplary element X variant
gtacttcatacttttcattcgggtggcttctgga

SEQ ID NO: 629 - DNA, 24 bp, Artificial sequence
  Exemplary element X variant
gtactcttttcaatggcttctgga

SEQ ID NO: 630 - DNA, 29 bp, Artificial sequence
  Exemplary element X variant
gtactcttttcaatggggtggcttctgga

SEQ ID NO: 631 - DNA, 29 bp, Artificial sequence
  Exemplary element X variant
gtactcttttcattcactttgcttctgga

SEQ ID NO: 632 - DNA, 29 bp, Artificial sequence
  Exemplary element X variant
gtacttcatacattcgggtggcttctgga

SEQ ID NO: 633 - DNA, 44 bp, Artificial sequence; identical to SEQ ID NO: 6
  PPE-1-derived regulatory element scheme

SEQ ID NO: 634 - DNA, 35 bp, Artificial sequence
  PPE-1-derived regulatory element scheme
gtacttcatacttttcattccaatggggtgacttt

SEQ ID NO: 635 - DNA, 25 bp, Artificial sequence
  PPE-1-derived regulatory element scheme
cattccaatggggtgactttgcttc

SEQ ID NO: 636 - DNA, 29 bp, Artificial sequence
  PPE-1-derived regulatory element scheme
cattccaatggggtgactttgcttctgga

SEQ ID NO: 637 - DNA, 20 bp, Artificial sequence
  PPE-1-derived regulatory element scheme
cattccaatggggtgacttt

SEQ ID NO: 638 - DNA, 14 bp, Artificial sequence
  PPE-1-derived regulatory element scheme
actttgcttctgga

SEQ ID NO: 639 - DNA, 10 bp, Artificial sequence
  PPE-1-derived regulatory element scheme
actttgcttc

SEQ ID NO: 640 - DNA, 9 bp, Artificial sequence
  PPE-1-derived regulatory element scheme
gcttctgga

SEQ ID NO: 641 - DNA, 14 bp, Artificial sequence
  PPE-1-derived regulatory element scheme
actttgcttctgga

SEQ ID NO: 642 - DNA, 10 bp, Artificial sequence
  PPE-1-derived regulatory element scheme
actttgcttc

SEQ ID NO: 643 - DNA, 9 bp, Artificial sequence
  PPE-1-derived regulatory element scheme
gcttctgga

SEQ ID NO: 644 - DNA, 15 bp, Artificial sequence
  PPE-1-derived regulatory element scheme
cattccaatggcttc

SEQ ID NO: 645 - DNA, 20 bp, Artificial sequence
  Single strand DNA oligonucleotide
ctcttgattcttgaactctg

SEQ ID NO: 646 - DNA, 20 bp, Artificial sequence
  Single strand DNA oligonucleotide
tacaagtaggttcctttgtg